SEQUENCE LISTING (389 sequences)

SEQ ID NO: 1 - protein, 251 aa, Homo sapiens
MGSSHHHHHHSSGLVPRGSHMPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 2 - protein, 25 aa, Artificial Sequence
  Description of Artificial Sequence: Synthetic peptide
LERNNIKQAANNSLLLHLLKSQTIP

SEQ ID NO: 3 - protein, 15 aa, Artificial Sequence
  Description of Artificial Sequence: Synthetic peptide
RHKILHRLLQEGSPS

SEQ ID NO: 4 - protein, 13 aa, Artificial Sequence
  Description of Artificial Sequence: Synthetic peptide
  N-term H
KHKILHRLLQDSS

SEQ ID NO: 5 - protein, 13 aa, Artificial Sequence
  Description of Artificial Sequence: Synthetic peptide
SGSHKLVQLLTTT

SEQ ID NO: 6 - protein, 21 aa, Artificial Sequence
  Description of Artificial Sequence: Synthetic peptide
TNMGLEAIRKALMGKYDQWEE

SEQ ID NO: 7 - protein, 14 aa, Artificial Sequence
  Description of Artificial Sequence: Synthetic peptide
QAQQKSLLQQLLTE

SEQ ID NO: 8 - protein, 19 aa, Artificial Sequence
  Description of Artificial Sequence: Synthetic peptide
LTERHKILHRLLQEGSPSD

SEQ ID NO: 9 - protein, 9 aa, Artificial Sequence
  Description of Artificial Sequence: Synthetic peptide
  MOD_RES (2)..(2): D-Cys
  misc_feature (1)..(9): Cyclic
KCILCRLLN

SEQ ID NO: 10 - protein, 15 aa, Homo sapiens
GGSCPSSHSSLTERH

SEQ ID NO: 11 - protein, 22 aa, Artificial Sequence
  Description of Artificial Sequence: Synthetic peptide
QQQKPQRRPCSELLKYLTTNDD

SEQ ID NO: 12 - protein, 12 aa, Artificial Sequence
  Description of Artificial Sequence: Synthetic peptide
KENALLKYLLDK

SEQ ID NO: 13 - protein, 22 aa, Homo sapiens
QQQKPQRRPCSELLKYLTTNDD

SEQ ID NO: 14 - protein, 12 aa, Homo sapiens
RPASELLKYLTT

SEQ ID NO: 15 - protein, 19 aa, Artificial Sequence
  Description of Artificial Sequence: Synthetic peptide
DEENEANLLAVLTETLDSL

SEQ ID NO: 16 - protein, 19 aa, Artificial Sequence
  Description of Artificial Sequence: Synthetic peptide
EAEEPSLLKKLLLAPANTQ

SEQ ID NO: 17 - protein, 19 aa, Artificial Sequence
  Description of Artificial Sequence: Synthetic peptide
QRRPCSELLKYLTTNDDPP

SEQ ID NO: 18 - protein, 19 aa, Artificial Sequence
  Description of Artificial Sequence: Synthetic peptide
DSKGQTKLLQLLTTKSDQM

SEQ ID NO: 19 - protein, 19 aa, Artificial Sequence
  Description of Artificial Sequence: Synthetic peptide
LKEKHKILHRLLQDSSSPV

SEQ ID NO: 20 - protein, 19 aa, Artificial Sequence
  Description of Artificial Sequence: Synthetic peptide
PKKKENALLRYLLDKDDTK

SEQ ID NO: 21 - protein, 12 aa, Artificial Sequence
  Description of Artificial Sequence: Synthetic peptide
KENALLKYLLDK

SEQ ID NO: 22 - protein, 19 aa, Artificial Sequence
  Description of Artificial Sequence: Synthetic peptide
PSEDRGELWRLLSVTERQN

SEQ ID NO: 23 - protein, 19 aa, Artificial Sequence
  Description of Artificial Sequence: Synthetic peptide
MSVQYPELQRLLMAGSTEL

SEQ ID NO: 24 - protein, 19 aa, Artificial Sequence
  Description of Artificial Sequence: Synthetic peptide
IPLSGSELSRLLLTEMPEL

SEQ ID NO: 25 - protein, 16 aa, Artificial Sequence
  Description of Artificial Sequence: Synthetic peptide
RRPCSELLKYLTTNDD

SEQ ID NO: 26 - protein, 19 aa, Artificial Sequence
  Description of Artificial Sequence: Synthetic peptide
IPLSGSELSRLLLTEMPEL

SEQ ID NO: 27 - protein, 19 aa, Artificial Sequence
  Description of Artificial Sequence: Synthetic peptide
IPVTDSELKRLLLEEVYER

SEQ ID NO: 28 - protein, 19 aa, Artificial Sequence
  Description of Artificial Sequence: Synthetic peptide
SGPEFTELYKLLISERDQV

SEQ ID NO: 29 - protein, 17 aa, Artificial Sequence
  Description of Artificial Sequence: Synthetic peptide
SELLHLLSHNIDSVSYS

SEQ ID NO: 30 - protein, 19 aa, Artificial Sequence
  Description of Artificial Sequence: Synthetic peptide
GVPDGGELRRLLEMDLPEV

SEQ ID NO: 31 - protein, 19 aa, Artificial Sequence
  Description of Artificial Sequence: Synthetic peptide
PSEDRGELWRLLSVTERQN

SEQ ID NO: 32 - protein, 19 aa, Artificial Sequence
  Description of Artificial Sequence: Synthetic peptide
VSHYPELLAQLLEGDVGLR

SEQ ID NO: 33 - protein, 19 aa, Artificial Sequence
  Description of Artificial Sequence: Synthetic peptide
NVQMFPELSRLLEKPEENL

SEQ ID NO: 34 - protein, 19 aa, Artificial Sequence
  Description of Artificial Sequence: Synthetic peptide
MSVQYPELQRLLMAGSTEL

SEQ ID NO: 35 - protein, 16 aa, Artificial Sequence
  Description of Artificial Sequence: Synthetic peptide
DAFQLRQLILRGLQDD

SEQ ID NO: 36 - protein, 19 aa, Artificial Sequence
  Description of Artificial Sequence: Synthetic peptide
GPQTPQAQQKSLLQQLLTE

SEQ ID NO: 37 - protein, 19 aa, Artificial Sequence
  Description of Artificial Sequence: Synthetic peptide
EAEEPSLLKKLLLAPANTQ

SEQ ID NO: 38 - protein, 19 aa, Artificial Sequence
  Description of Artificial Sequence: Synthetic peptide
LPYEGSLLLKLLRAPVEEV

SEQ ID NO: 39 - protein, 21 aa, Artificial Sequence
  Description of Artificial Sequence: Synthetic peptide
KYSQTSHKLVQLLTTTAEQQL

SEQ ID NO: 40 - protein, 19 aa, Artificial Sequence
  Description of Artificial Sequence: Synthetic peptide
LTARHKILHRLLQEGSPSD

SEQ ID NO: 41 - protein, 19 aa, Artificial Sequence
  Description of Artificial Sequence: Synthetic peptide
ESKDHQLLRYLLDKDEKDL

SEQ ID NO: 42 - protein, 19 aa, Artificial Sequence
  Description of Artificial Sequence: Synthetic peptide
GPQTPQAQQKSLLQQLLTE

SEQ ID NO: 43 - protein, 19 aa, Artificial Sequence
  Description of Artificial Sequence: Synthetic peptide
DSKGQTKLLQLLTTKSDQM

SEQ ID NO: 44 - protein, 19 aa, Artificial Sequence
  Description of Artificial Sequence: Synthetic peptide
LKEKHKILHRLLQDSSSPV

SEQ ID NO: 45 - protein, 19 aa, Artificial Sequence
  Description of Artificial Sequence: Synthetic peptide
KKKENALLRYLLDKDDTKD

SEQ ID NO: 46 - protein, 19 aa, Artificial Sequence
  Description of Artificial Sequence: Synthetic peptide
ESKGHKKLLQLLTCSSDDR

SEQ ID NO: 47 - protein, 19 aa, Artificial Sequence
  Description of Artificial Sequence: Synthetic peptide
LQEKHRILHKLLQNGNSPA

SEQ ID NO: 48 - protein, 19 aa, Artificial Sequence
  Description of Artificial Sequence: Synthetic peptide
KKENNALLRYLLDRDDPSD

SEQ ID NO: 49 - protein, 19 aa, Artificial Sequence
  Description of Artificial Sequence: Synthetic peptide
AASKHKQLSELLRGGSGSS

SEQ ID NO: 50 - protein, 19 aa, Artificial Sequence
  Description of Artificial Sequence: Synthetic peptide
KVSQNPILTSLLQITGNGG

SEQ ID NO: 51 - protein, 19 aa, Artificial Sequence
  Description of Artificial Sequence: Synthetic peptide
NTKNHPMLMNLLKDNPAQD

SEQ ID NO: 52 - protein, 19 aa, Artificial Sequence
  Description of Artificial Sequence: Synthetic peptide
SHQKVTLLQLLLGHKNEEN

SEQ ID NO: 53 - protein, 19 aa, Artificial Sequence
  Description of Artificial Sequence: Synthetic peptide
SFSKNGLLSRLLRQNQDSY

SEQ ID NO: 54 - protein, 19 aa, Artificial Sequence
  Description of Artificial Sequence: Synthetic peptide
EAEEPSLLKKLLLAPANTQ

SEQ ID NO: 55 - protein, 19 aa, Artificial Sequence
  Description of Artificial Sequence: Synthetic peptide
VTLTSPLLVNLLQSDISAG

SEQ ID NO: 56 - protein, 19 aa, Artificial Sequence
  Description of Artificial Sequence: Synthetic peptide
LPYEGSLLLKLLRAPVEEV

SEQ ID NO: 57 - protein, 19 aa, Artificial Sequence
  Description of Artificial Sequence: Synthetic peptide
HVEMHPLLMGLLMESQWGA

SEQ ID NO: 58 - protein, 15 aa, Artificial Sequence
  Description of Artificial Sequence: Synthetic peptide
SSNHQSSRLIELLSR

SEQ ID NO: 59 - protein, 16 aa, Artificial Sequence
  Description of Artificial Sequence: Synthetic peptide
SSKGVLWRMLAEPVSR

SEQ ID NO: 60 - protein, 15 aa, Artificial Sequence
  Description of Artificial Sequence: Synthetic peptide
SRTLQLDWGTLYWSR

SEQ ID NO: 61 - protein, 15 aa, Artificial Sequence
  Description of Artificial Sequence: Synthetic peptide
SSVASREWWVRELSR

SEQ ID NO: 62 - protein, 19 aa, Artificial Sequence
  Description of Artificial Sequence: Synthetic peptide
SKTYRGAFQNLFQSVREVI

SEQ ID NO: 63 - protein, 19 aa, Artificial Sequence
  Description of Artificial Sequence: Synthetic peptide
SRETSEKFKLLFQSYNVND

SEQ ID NO: 64 - protein, 19 aa, Artificial Sequence
  Description of Artificial Sequence: Synthetic peptide
VESGSSRFMQLFMANDLLT

SEQ ID NO: 65 - protein, 23 aa, Artificial Sequence
  Description of Artificial Sequence: Synthetic peptide
GHQRVVTLAQHISEVITQDYTRH

SEQ ID NO: 66 - protein, 23 aa, Artificial Sequence
  Description of Artificial Sequence: Synthetic peptide
HASTNMGLEAIIRKALMGKYDQW

SEQ ID NO: 67 - protein, 14 aa, Artificial Sequence
  Description of Artificial Sequence: Synthetic peptide
NLGLEDIIRKALMG

SEQ ID NO: 68 - protein, 13 aa, Artificial Sequence
  Description of Artificial Sequence: Synthetic peptide
ASHKQLSELLRGG

SEQ ID NO: 69 - protein, 16 aa, Artificial Sequence
  Description of Artificial Sequence: Synthetic peptide
SLTARHKILHRLLQEG

SEQ ID NO: 70 - protein, 15 aa, Artificial Sequence
  Description of Artificial Sequence: Synthetic peptide
RHKILHRLLQEGSPS

SEQ ID NO: 71 - protein, 21 aa, Artificial Sequence
  Description of Artificial Sequence: Synthetic peptide
SLTERHKILHRLLQEGSPSDI

SEQ ID NO: 72 - protein, 12 aa, Homo sapiens
RPCSELLKYLTT

SEQ ID NO: 73 - protein, 11 aa, Artificial Sequence
  Description of Artificial Sequence: Synthetic peptide
RGAFQNLFQSV

SEQ ID NO: 74 - protein, 14 aa, Artificial Sequence
  Description of Artificial Sequence: Synthetic peptide
KENALLRYLLDKDD

SEQ ID NO: 75 - protein, 14 aa, Artificial Sequence
  Description of Artificial Sequence: Synthetic peptide
EKHKILHRLLQDSY

SEQ ID NO: 76 - protein, 7 aa, Artificial Sequence
  Description of Artificial Sequence: Synthetic peptide
  MOD_RES (4)..(4): Any amino acid
PCFXILP

SEQ ID NO: 77 - protein, 17 aa, Artificial Sequence
  Description of Artificial Sequence: Synthetic peptide
CGSGKHKILHRLLQDSS

SEQ ID NO: 78 - protein, 17 aa, Artificial Sequence
  Description of Artificial Sequence: Synthetic peptide
CGSGQGSILYSLLTSSK

SEQ ID NO: 79 - protein, 23 aa, Artificial Sequence
  Description of Artificial Sequence: Synthetic peptide
AENQRGPLESKGHKKLLQLLTSS

SEQ ID NO: 80 - protein, 13 aa, Artificial Sequence
  Description of Artificial Sequence: Synthetic peptide
KHKILHRLLQDSS

SEQ ID NO: 81 - protein, 13 aa, Artificial Sequence
  Description of Artificial Sequence: Synthetic peptide
KHKILHRLLQDSS

SEQ ID NO: 82 - protein, 10 aa, Artificial Sequence
  Description of Artificial Sequence: Synthetic peptide
CQQQKPQRRP

SEQ ID NO: 83 - protein, 14 aa, Artificial Sequence
  Description of Artificial Sequence: Synthetic peptide
LTKTNPILYYMLQK

SEQ ID NO: 84 - protein, 15 aa, Artificial Sequence
  Description of Artificial Sequence: Synthetic peptide
LPALDPTKRWFFETK

SEQ ID NO: 85 - protein, 19 aa, Artificial Sequence
  Description of Artificial Sequence: Synthetic peptide
TGGGVSLLLHLLNTEQGES

SEQ ID NO: 86 - protein, 19 aa, Artificial Sequence
  Description of Artificial Sequence: Synthetic peptide
RRDDFPLLISLLKDGALSQ

SEQ ID NO: 87 - protein, 19 aa, Artificial Sequence
  Description of Artificial Sequence: Synthetic peptide
YGLKMSLLESLLREDISTV

SEQ ID NO: 88 - protein, 19 aa, Artificial Sequence
  Description of Artificial Sequence: Synthetic peptide
MSYDMLSLYPLLTNSLLEV

SEQ ID NO: 89 - protein, 19 aa, Artificial Sequence
  Description of Artificial Sequence: Synthetic peptide
FPAEFPLLTYLLERQGMDE

SEQ ID NO: 90 - protein, 19 aa, Artificial Sequence
  Description of Artificial Sequence: Synthetic peptide
VESEFPYLLSLLGEVSPQP

SEQ ID NO: 91 - protein, 19 aa, Artificial Sequence
  Description of Artificial Sequence: Synthetic peptide
VSSEGRLLIDLLVDGQQSE

SEQ ID NO: 92 - protein, 19 aa, Artificial Sequence
  Description of Artificial Sequence: Synthetic peptide
DTPQSPLLWGLLSSDRVEG

SEQ ID NO: 93 - protein, 19 aa, Artificial Sequence
  Description of Artificial Sequence: Synthetic peptide
GGTQDGYLWSLLTGMPEVS

SEQ ID NO: 94 - protein, 19 aa, Artificial Sequence
  Description of Artificial Sequence: Synthetic peptide
SLPEEGFLMKLLTLEGDAE

SEQ ID NO: 95 - protein, 19 aa, Artificial Sequence
  Description of Artificial Sequence: Synthetic peptide
VMGNNPILVSLLEEPSEEP

SEQ ID NO: 96 - protein, 19 aa, Artificial Sequence
  Description of Artificial Sequence: Synthetic peptide
VLVEHPILGGLLSTRVDSS

SEQ ID NO: 97 - protein, 16 aa, Artificial Sequence
  Description of Artificial Sequence: Synthetic peptide
QTPLLEQLLTEHIQQG

SEQ ID NO: 98 - protein, 19 aa, Artificial Sequence
  Description of Artificial Sequence: Synthetic peptide
GSWQDSLLLQLLNRTELMA

SEQ ID NO: 99 - protein, 19 aa, Artificial Sequence
  Description of Artificial Sequence: Synthetic peptide
SVWPGPELLKLLSGTSVAE

SEQ ID NO: 100 - protein, 6 aa, Artificial Sequence
  Description of Artificial Sequence: Synthetic peptide
VSWFFE

SEQ ID NO: 101 - protein, 7 aa, Artificial Sequence
  Description of Artificial Sequence: Synthetic peptide
VSWFFED

SEQ ID NO: 102 - protein, 9 aa, Artificial Sequence
  Description of Artificial Sequence: Synthetic peptide
  N-term H
  MOD_RES (2)..(2): D-Cys
  misc_feature (2)..(5): Cyclic
  C-term NH2
KCILCRLLQ

SEQ ID NO: 103 - protein, 10 aa, Artificial Sequence
  Description of Artificial Sequence: Synthetic peptide
NIFSEVRVYN

SEQ ID NO: 104 - protein, 9 aa, Artificial Sequence
  Description of Artificial Sequence: Synthetic peptide
VLWKLLKVV

SEQ ID NO: 105 - protein, 15 aa, Artificial Sequence
  Description of Artificial Sequence: Synthetic peptide
SGSGLTSRDFGSWYA

SEQ ID NO: 106 - protein, 13 aa, Artificial Sequence
  Description of Artificial Sequence: Synthetic peptide
EKHKILHRLLQDS

SEQ ID NO: 107 - protein, 15 aa, Artificial Sequence
  Description of Artificial Sequence: Synthetic peptide
RHKILHRLLQEGSPS

SEQ ID NO: 108 - protein, 26 aa, Homo sapiens
  N-term Biotinyl
DGTPPPQEAEEPSLLKKLLLAPANTQ

SEQ ID NO: 109 - protein, 25 aa, Homo sapiens
  N-term Biotinyl
CPCQGAASRPAILYALLSSSLKAVP

SEQ ID NO: 110 - protein, 25 aa, Homo sapiens
  N-term Biotinyl
QEQLSPKKKENNALLRYLLDRDDPS

SEQ ID NO: 111 - protein, 27 aa, Homo sapiens
  N-term Biotinyl
PVSPQDFSFSKNGLLSRLLRQNQDSYL

SEQ ID NO: 112 - protein, 25 aa, Homo sapiens
  N-term Biotinyl
QEPVSPKKKENALLRYLLDKDDTKD

SEQ ID NO: 113 - protein, 21 aa, Homo sapiens
  N-term Biotinyl
TSGPQTPQAQQKSLLQQLLTE

SEQ ID NO: 114 - protein, 25 aa, Homo sapiens
  N-term Biotinyl
EHRSWARESKSFNVLKQLLSLENCV

SEQ ID NO: 115 - protein, 26 aa, Homo sapiens
  N-term Biotinyl
FCGEDHPRQGSILYSLLTSSKQTHVA

SEQ ID NO: 116 - protein, 25 aa, Homo sapiens
  N-term Biotinyl
LERNNIKQAANNSLLLHLLKSQTIP

SEQ ID NO: 117 - protein, 24 aa, Homo sapiens
  N-term Biotinyl
GEDHPRQGSILYSLLTSSKQTHVA

SEQ ID NO: 118 - protein, 25 aa, Homo sapiens
  N-term Biotinyl
CPSSHSSLTERHKILHRLLQEGSPS

SEQ ID NO: 119 - protein, 25 aa, Homo sapiens
  N-term Biotinyl
SNMHGSLLQEKHRILHKLLQNGNSP

SEQ ID NO: 120 - protein, 25 aa, Homo sapiens
  N-term Biotinyl
EKDLRCYGVASSHLKTLLKKSKVKD

SEQ ID NO: 121 - protein, 14 aa, Artificial Sequence
  Description of Artificial Sequence: Synthetic peptide
QAQQKSLLQQLLTE

SEQ ID NO: 122 - protein, 19 aa, Artificial Sequence
  Description of Artificial Sequence: Synthetic peptide
GPQTPQAQQKSLLQQLLTE

SEQ ID NO: 123 - protein, 25 aa, Artificial Sequence
  Description of Artificial Sequence: Synthetic peptide
LERNNIKQAANNSLLLHLLKSQTIP

SEQ ID NO: 124 - protein, 13 aa, Artificial Sequence
  Description of Artificial Sequence: Synthetic peptide
SGSHKLVQLLTTT

SEQ ID NO: 125 - protein, 23 aa, Artificial Sequence
  Description of Artificial Sequence: Synthetic peptide
AENQRGPLESKGHKKLLQLLTSS

SEQ ID NO: 126 - protein, 19 aa, Artificial Sequence
  Description of Artificial Sequence: Synthetic peptide
SHQKVTLLQLLLGHKNEEN

SEQ ID NO: 127 - protein, 17 aa, Artificial Sequence
  Description of Artificial Sequence: Synthetic peptide
CGSGQGSILYSLLTSSK

SEQ ID NO: 128 - protein, 12 aa, Artificial Sequence
  Description of Artificial Sequence: Synthetic peptide
KENALLKYLLDK

SEQ ID NO: 129 - protein, 12 aa, Artificial Sequence
  Description of Artificial Sequence: Synthetic peptide
KENALLRYLLDK

SEQ ID NO: 130 - protein, 14 aa, Artificial Sequence
  Description of Artificial Sequence: Synthetic peptide
LTKTNPILYYMLQK

SEQ ID NO: 131 - protein, 19 aa, Artificial Sequence
  Description of Artificial Sequence: Synthetic peptide
EAEEPSLLKKLLLAPANTQ

SEQ ID NO: 132 - protein, 19 aa, Artificial Sequence
  Description of Artificial Sequence: Synthetic peptide
LPYEGSLLLKLLRAPVEEV

SEQ ID NO: 133 - protein, 19 aa, Artificial Sequence
  Description of Artificial Sequence: Synthetic peptide
PSEDRGELWRLLSVTERQN

SEQ ID NO: 134 - protein, 9 aa, Artificial Sequence
  Description of Artificial Sequence: Synthetic peptide
VLWKLLKVV

SEQ ID NO: 135 - protein, 15 aa, Artificial Sequence
  Description of Artificial Sequence: Synthetic peptide
RHKILHRLLQEGSPS

SEQ ID NO: 136 - protein, 19 aa, Artificial Sequence
  Description of Artificial Sequence: Synthetic peptide
LTERHKILHRLLQEGSPSD

SEQ ID NO: 137 - protein, 21 aa, Artificial Sequence
  Description of Artificial Sequence: Synthetic peptide
SLTERHKILHRLLQEGSPSDI

SEQ ID NO: 138 - protein, 16 aa, Artificial Sequence
  Description of Artificial Sequence: Synthetic peptide
SLTARHKILHRLLQEG

SEQ ID NO: 139 - protein, 13 aa, Artificial Sequence
  Description of Artificial Sequence: Synthetic peptide
KHKILHRLLQDSS

SEQ ID NO: 140 - protein, 17 aa, Artificial Sequence
  Description of Artificial Sequence: Synthetic peptide
CGSGKHKILHRLLQDSS

SEQ ID NO: 141 - protein, 13 aa, Artificial Sequence
  Description of Artificial Sequence: Synthetic peptide
KHKILHRLLQDSS

SEQ ID NO: 142 - protein, 14 aa, Artificial Sequence
  Description of Artificial Sequence: Synthetic peptide
EKHKILHRLLQDSY

SEQ ID NO: 143 - protein, 10 aa, Artificial Sequence
  Description of Artificial Sequence: Synthetic peptide
KDCILCRLLN

SEQ ID NO: 144 - protein, 19 aa, Artificial Sequence
  Description of Artificial Sequence: Synthetic peptide
SFSKNGLLSRLLRQNQDSY

SEQ ID NO: 145 - protein, 13 aa, Artificial Sequence
  Description of Artificial Sequence: Synthetic peptide
ASHKQLSELLRGG

SEQ ID NO: 146 - protein, 19 aa, Artificial Sequence
  Description of Artificial Sequence: Synthetic peptide
MSVQYPELQRLLMAGSTEL

SEQ ID NO: 147 - protein, 19 aa, Artificial Sequence
  Description of Artificial Sequence: Synthetic peptide
IPLSGSELSRLLLTEMPEL

SEQ ID NO: 148 - protein, 22 aa, Artificial Sequence
  Description of Artificial Sequence: Synthetic peptide
QQQKPQRRPCSELLKYLTTNDD

SEQ ID NO: 149 - protein, 10 aa, Artificial Sequence
  Description of Artificial Sequence: Synthetic peptide
CQQQKPQRRP

SEQ ID NO: 150 - protein, 12 aa, Artificial Sequence
  Description of Artificial Sequence: Synthetic peptide
RPCSELLKYLTT

SEQ ID NO: 151 - protein, 12 aa, Artificial Sequence
  Description of Artificial Sequence: Synthetic peptide
RPCSELLKYLTT

SEQ ID NO: 152 - protein, 15 aa, Artificial Sequence
  Description of Artificial Sequence: Synthetic peptide
LPALDPTKRWFFETK

SEQ ID NO: 153 - protein, 7 aa, Artificial Sequence
  Description of Artificial Sequence: Synthetic peptide
VSWFFED

SEQ ID NO: 154 - protein, 15 aa, Artificial Sequence
  Description of Artificial Sequence: Synthetic peptide
GGSCPSSHSSLTERH

SEQ ID NO: 155 - protein, 16 aa, Artificial Sequence
  Description of Artificial Sequence: Synthetic peptide
DAFQLRQLILRGLQDD

SEQ ID NO: 156 - protein, 11 aa, Artificial Sequence
  Description of Artificial Sequence: Synthetic peptide
RGAFQNLFQSV

SEQ ID NO: 157 - skipped

SEQ ID NO: 158 - protein, 10 aa, Artificial Sequence
  Description of Artificial Sequence: Synthetic peptide
NIFSEVRVYN

SEQ ID NO: 159 - protein, 21 aa, Artificial Sequence
  Description of Artificial Sequence: Synthetic peptide
TNMGLEAIRKALMGKYDQWEE

SEQ ID NO: 160 - protein, 8 aa, Artificial Sequence
  Description of Artificial Sequence: Synthetic peptide
NSLLQHLL

SEQ ID NO: 161 - protein, 8 aa, Artificial Sequence
  Description of Artificial Sequence: Synthetic peptide
NSLKKHLL

SEQ ID NO: 162 - protein, 8 aa, Artificial Sequence
  Description of Artificial Sequence: Synthetic peptide
ANNSQLLH

SEQ ID NO: 163 - protein, 8 aa, Artificial Sequence
  Description of Artificial Sequence: Synthetic peptide
IKQNHNNS

SEQ ID NO: 164 - protein, 8 aa, Artificial Sequence
  Description of Artificial Sequence: Synthetic peptide
IKQDHNNS

SEQ ID NO: 165 - protein, 8 aa, Artificial Sequence
  Description of Artificial Sequence: Synthetic peptide
ANNIQLLH

SEQ ID NO: 166 - protein, 8 aa, Artificial Sequence
  Description of Artificial Sequence: Synthetic peptide
NSLLKHLL

SEQ ID NO: 167 - protein, 8 aa, Artificial Sequence
  Description of Artificial Sequence: Synthetic peptide
NIKVNANN

SEQ ID NO: 168 - protein, 8 aa, Artificial Sequence
  Description of Artificial Sequence: Synthetic peptide
AANYLLLL

SEQ ID NO: 169 - protein, 8 aa, Artificial Sequence
  Description of Artificial Sequence: Synthetic peptide
ANNSSLLH

SEQ ID NO: 170 - protein, 8 aa, Artificial Sequence
  Description of Artificial Sequence: Synthetic peptide
AANYVLLL

SEQ ID NO: 171 - protein, 8 aa, Artificial Sequence
  Description of Artificial Sequence: Synthetic peptide
AANYILLL

SEQ ID NO: 172 - protein, 8 aa, Artificial Sequence
  Description of Artificial Sequence: Synthetic peptide
AANNYLLL

SEQ ID NO: 173 - protein, 8 aa, Artificial Sequence
  Description of Artificial Sequence: Synthetic peptide
NIKVHANN

SEQ ID NO: 174 - protein, 8 aa, Artificial Sequence
  Description of Artificial Sequence: Synthetic peptide
ANNSLLLH

SEQ ID NO: 175 - protein, 8 aa, Artificial Sequence
  Description of Artificial Sequence: Synthetic peptide
LHLKGSQT

SEQ ID NO: 176 - protein, 8 aa, Artificial Sequence
  Description of Artificial Sequence: Synthetic peptide
KSLLQQLL

SEQ ID NO: 177 - protein, 8 aa, Artificial Sequence
  Description of Artificial Sequence: Synthetic peptide
NSLRSHLL

SEQ ID NO: 178 - protein, 8 aa, Artificial Sequence
  Description of Artificial Sequence: Synthetic peptide
ANNILLLH

SEQ ID NO: 179 - protein, 8 aa, Artificial Sequence
  Description of Artificial Sequence: Synthetic peptide
AANMSLLL

SEQ ID NO: 180 - protein, 8 aa, Artificial Sequence
  Description of Artificial Sequence: Synthetic peptide
NSLRQHLL

SEQ ID NO: 181 - protein, 8 aa, Artificial Sequence
  Description of Artificial Sequence: Synthetic peptide
ANNSWLLH

SEQ ID NO: 182 - protein, 8 aa, Artificial Sequence
  Description of Artificial Sequence: Synthetic peptide
AANMYLLL

SEQ ID NO: 183 - protein, 8 aa, Artificial Sequence
  Description of Artificial Sequence: Synthetic peptide
AIRVHLMG

SEQ ID NO: 184 - protein, 8 aa, Artificial Sequence
  Description of Artificial Sequence: Synthetic peptide
ANNLSLLH

SEQ ID NO: 185 - protein, 8 aa, Artificial Sequence
  Description of Artificial Sequence: Synthetic peptide
LLHLQKSQ

SEQ ID NO: 186 - protein, 8 aa, Artificial Sequence
  Description of Artificial Sequence: Synthetic peptide
AIRGHLMG

SEQ ID NO: 187 - protein, 8 aa, Artificial Sequence
  Description of Artificial Sequence: Synthetic peptide
ANNSRLLH

SEQ ID NO: 188 - protein, 8 aa, Artificial Sequence
  Description of Artificial Sequence: Synthetic peptide
AANMLLLL

SEQ ID NO: 189 - protein, 8 aa, Artificial Sequence
  Description of Artificial Sequence: Synthetic peptide
AIRGALMG

SEQ ID NO: 190 - protein, 8 aa, Artificial Sequence
  Description of Artificial Sequence: Synthetic peptide
ANNSKLLH

SEQ ID NO: 191 - protein, 8 aa, Artificial Sequence
  Description of Artificial Sequence: Synthetic peptide
LLHSSKSQ

SEQ ID NO: 192 - protein, 8 aa, Artificial Sequence
  Description of Artificial Sequence: Synthetic peptide
AANMFLLL

SEQ ID NO: 193 - protein, 8 aa, Artificial Sequence
  Description of Artificial Sequence: Synthetic peptide
NSLRKHLL

SEQ ID NO: 194 - protein, 8 aa, Artificial Sequence
  Description of Artificial Sequence: Synthetic peptide
NSLSWHLL

SEQ ID NO: 195 - protein, 8 aa, Artificial Sequence
  Description of Artificial Sequence: Synthetic peptide
ANNLWLLH

SEQ ID NO: 196 - protein, 8 aa, Artificial Sequence
  Description of Artificial Sequence: Synthetic peptide
ANNFLLLH

SEQ ID NO: 197 - protein, 8 aa, Artificial Sequence
  Description of Artificial Sequence: Synthetic peptide
RKAIFGKY

SEQ ID NO: 198 - protein, 8 aa, Artificial Sequence
  Description of Artificial Sequence: Synthetic peptide
AIRLHLMG

SEQ ID NO: 199 - protein, 8 aa, Artificial Sequence
  Description of Artificial Sequence: Synthetic peptide
ANNIWLLH

SEQ ID NO: 200 - protein, 8 aa, Artificial Sequence
  Description of Artificial Sequence: Synthetic peptide
ANNVKLLH

SEQ ID NO: 201 - protein, 8 aa, Artificial Sequence
  Description of Artificial Sequence: Synthetic peptide
ANNLQLLH

SEQ ID NO: 202 - protein, 8 aa, Artificial Sequence
  Description of Artificial Sequence: Synthetic peptide
LLHKSKSQ

SEQ ID NO: 203 - protein, 8 aa, Artificial Sequence
  Description of Artificial Sequence: Synthetic peptide
IKQNYNNS

SEQ ID NO: 204 - protein, 8 aa, Artificial Sequence
  Description of Artificial Sequence: Synthetic peptide
NNSWSLHL

SEQ ID NO: 205 - protein, 8 aa, Artificial Sequence
  Description of Artificial Sequence: Synthetic peptide
RKALVGKY

SEQ ID NO: 206 - protein, 8 aa, Artificial Sequence
  Description of Artificial Sequence: Synthetic peptide
ANNVLLLH

SEQ ID NO: 207 - protein, 8 aa, Artificial Sequence
  Description of Artificial Sequence: Synthetic peptide
ANNLKLLH

SEQ ID NO: 208 - protein, 8 aa, Artificial Sequence
  Description of Artificial Sequence: Synthetic peptide
ANNVSLLH

SEQ ID NO: 209 - protein, 8 aa, Artificial Sequence
  Description of Artificial Sequence: Synthetic peptide
LHLRYSQT

SEQ ID NO: 210 - protein, 8 aa, Artificial Sequence
  Description of Artificial Sequence: Synthetic peptide
HDNMYLNE

SEQ ID NO: 211 - protein, 8 aa, Artificial Sequence
  Description of Artificial Sequence: Synthetic peptide
DINFSLGI

SEQ ID NO: 212 - protein, 8 aa, Artificial Sequence
  Description of Artificial Sequence: Synthetic peptide
NFNYLLPH

SEQ ID NO: 213 - protein, 8 aa, Artificial Sequence
  Description of Artificial Sequence: Synthetic peptide
HRSYPRRG

SEQ ID NO: 214 - protein, 8 aa, Artificial Sequence
  Description of Artificial Sequence: Synthetic peptide
NNNFSLHM

SEQ ID NO: 215 - protein, 8 aa, Artificial Sequence
  Description of Artificial Sequence: Synthetic peptide
PDSYPRWG

SEQ ID NO: 216 - protein, 8 aa, Artificial Sequence
  Description of Artificial Sequence: Synthetic peptide
EDSYPRDG

SEQ ID NO: 217 - protein, 8 aa, Artificial Sequence
  Description of Artificial Sequence: Synthetic peptide
WANYLLPP

SEQ ID NO: 218 - protein, 8 aa, Artificial Sequence
  Description of Artificial Sequence: Synthetic peptide
DNNYLLLL

SEQ ID NO: 219 - protein, 8 aa, Artificial Sequence
  Description of Artificial Sequence: Synthetic peptide
WIKLKQND

SEQ ID NO: 220 - protein, 8 aa, Artificial Sequence
  Description of Artificial Sequence: Synthetic peptide
TGKLKQDG

SEQ ID NO: 221 - protein, 8 aa, Artificial Sequence
  Description of Artificial Sequence: Synthetic peptide
LFLLQQDD

SEQ ID NO: 222 - protein, 8 aa, Artificial Sequence
  Description of Artificial Sequence: Synthetic peptide
NLLLKHPD

SEQ ID NO: 223 - protein, 8 aa, Artificial Sequence
  Description of Artificial Sequence: Synthetic peptide
FFKGHFKF

SEQ ID NO: 224 - protein, 8 aa, Artificial Sequence
  Description of Artificial Sequence: Synthetic peptide
LFKLKQDG

SEQ ID NO: 225 - protein, 8 aa, Artificial Sequence
  Description of Artificial Sequence: Synthetic peptide
HLKLKQEN

SEQ ID NO: 226 - protein, 8 aa, Artificial Sequence
  Description of Artificial Sequence: Synthetic peptide
LYLLKHHP

SEQ ID NO: 227 - protein, 8 aa, Artificial Sequence
  Description of Artificial Sequence: Synthetic peptide
HYKLKQHE

SEQ ID NO: 228 - protein, 8 aa, Artificial Sequence
  Description of Artificial Sequence: Synthetic peptide
WNSYPRND

SEQ ID NO: 229 - protein, 8 aa, Artificial Sequence
  Description of Artificial Sequence: Synthetic peptide
LILLQQLD

SEQ ID NO: 230 - protein, 8 aa, Artificial Sequence
  Description of Artificial Sequence: Synthetic peptide
PWKLKQDP

SEQ ID NO: 231 - protein, 8 aa, Artificial Sequence
  Description of Artificial Sequence: Synthetic peptide
FWKGHFDF

SEQ ID NO: 232 - protein, 8 aa, Artificial Sequence
  Description of Artificial Sequence: Synthetic peptide
FFNFSLPA

SEQ ID NO: 233 - protein, 8 aa, Artificial Sequence
  Description of Artificial Sequence: Synthetic peptide
FYNMYLLN

SEQ ID NO: 234 - protein, 8 aa, Artificial Sequence
  Description of Artificial Sequence: Synthetic peptide
WGKLKQGE

SEQ ID NO: 235 - protein, 8 aa, Artificial Sequence
  Description of Artificial Sequence: Synthetic peptide
HNSYPRNE

SEQ ID NO: 236 - protein, 8 aa, Artificial Sequence
  Description of Artificial Sequence: Synthetic peptide
GKSYPRHR

SEQ ID NO: 237 - protein, 8 aa, Artificial Sequence
  Description of Artificial Sequence: Synthetic peptide
YHNYLLHP

SEQ ID NO: 238 - protein, 8 aa, Artificial Sequence
  Description of Artificial Sequence: Synthetic peptide
AHNYLLEH

SEQ ID NO: 239 - protein, 8 aa, Artificial Sequence
  Description of Artificial Sequence: Synthetic peptide
VLLHYGLY

SEQ ID NO: 240 - protein, 8 aa, Artificial Sequence
  Description of Artificial Sequence: Synthetic peptide
LFKLKQND

SEQ ID NO: 241 - protein, 8 aa, Artificial Sequence
  Description of Artificial Sequence: Synthetic peptide
HNNYLLNN

SEQ ID NO: 242 - protein, 8 aa, Artificial Sequence
  Description of Artificial Sequence: Synthetic peptide
HRSYPRVY

SEQ ID NO: 243 - protein, 8 aa, Artificial Sequence
  Description of Artificial Sequence: Synthetic peptide
FWNMYLNP

SEQ ID NO: 244 - protein, 8 aa, Artificial Sequence
  Description of Artificial Sequence: Synthetic peptide
HFKLKQGG

SEQ ID NO: 245 - protein, 8 aa, Artificial Sequence
  Description of Artificial Sequence: Synthetic peptide
NNSYPRHR

SEQ ID NO: 246 - protein, 8 aa, Artificial Sequence
  Description of Artificial Sequence: Synthetic peptide
PFKLKQGN

SEQ ID NO: 247 - protein, 8 aa, Artificial Sequence
  Description of Artificial Sequence: Synthetic peptide
EESYPRDK

SEQ ID NO: 248 - protein, 8 aa, Artificial Sequence
  Description of Artificial Sequence: Synthetic peptide
ALNFSLNW

SEQ ID NO: 249 - protein, 8 aa, Artificial Sequence
  Description of Artificial Sequence: Synthetic peptide
IFLLQQDN

SEQ ID NO: 250 - protein, 8 aa, Artificial Sequence
  Description of Artificial Sequence: Synthetic peptide
WRSYPRRA

SEQ ID NO: 251 - protein, 8 aa, Artificial Sequence
  Description of Artificial Sequence: Synthetic peptide
RLLHYGSY

SEQ ID NO: 252 - protein, 8 aa, Artificial Sequence
  Description of Artificial Sequence: Synthetic peptide
HNNFSLEP

SEQ ID NO: 253 - protein, 8 aa, Artificial Sequence
  Description of Artificial Sequence: Synthetic peptide
MYKLKQDN

SEQ ID NO: 254 - protein, 8 aa, Artificial Sequence
  Description of Artificial Sequence: Synthetic peptide
WLLLKHGL

SEQ ID NO: 255 - protein, 8 aa, Artificial Sequence
  Description of Artificial Sequence: Synthetic peptide
DRSYPRHT

SEQ ID NO: 256 - protein, 8 aa, Artificial Sequence
  Description of Artificial Sequence: Synthetic peptide
YFLLQQGP

SEQ ID NO: 257 - protein, 8 aa, Artificial Sequence
  Description of Artificial Sequence: Synthetic peptide
PGKGHFPI

SEQ ID NO: 258 - protein, 8 aa, Artificial Sequence
  Description of Artificial Sequence: Synthetic peptide
AFNMYLDE

SEQ ID NO: 259 - protein, 8 aa, Artificial Sequence
  Description of Artificial Sequence: Synthetic peptide
FVNFSLGI

SEQ ID NO: 260 - protein, 8 aa, Artificial Sequence
  Description of Artificial Sequence: Synthetic peptide
RRSYPRGR

SEQ ID NO: 261 - protein, 8 aa, Artificial Sequence
  Description of Artificial Sequence: Synthetic peptide
IFSEMRVY

SEQ ID NO: 262 - protein, 8 aa, Artificial Sequence
  Description of Artificial Sequence: Synthetic peptide
FWKGHFDF

SEQ ID NO: 263 - protein, 8 aa, Artificial Sequence
  Description of Artificial Sequence: Synthetic peptide
IKQNHNNS

SEQ ID NO: 264 - protein, 8 aa, Artificial Sequence
  Description of Artificial Sequence: Synthetic peptide
AIRLFLMG

SEQ ID NO: 265 - protein, 8 aa, Artificial Sequence
  Description of Artificial Sequence: Synthetic peptide
IFSYVRVY

SEQ ID NO: 266 - protein, 8 aa, Artificial Sequence
  Description of Artificial Sequence: Synthetic peptide
IFSKIRVY

SEQ ID NO: 267 - protein, 8 aa, Artificial Sequence
  Description of Artificial Sequence: Synthetic peptide
IKQDHNNS

SEQ ID NO: 268 - protein, 8 aa, Artificial Sequence
  Description of Artificial Sequence: Synthetic peptide
IFSEIRVY

SEQ ID NO: 269 - protein, 8 aa, Artificial Sequence
  Description of Artificial Sequence: Synthetic peptide
NIKVNANN

SEQ ID NO: 270 - protein, 8 aa, Artificial Sequence
  Description of Artificial Sequence: Synthetic peptide
IFSYPRVY

SEQ ID NO: 271 - protein, 8 aa, Artificial Sequence
  Description of Artificial Sequence: Synthetic peptide
QYPKMQRL

SEQ ID NO: 272 - protein, 8 aa, Artificial Sequence
  Description of Artificial Sequence: Synthetic peptide
NSLKKHLL

SEQ ID NO: 273 - protein, 8 aa, Artificial Sequence
  Description of Artificial Sequence: Synthetic peptide
ANNIRLLH

SEQ ID NO: 274 - protein, 8 aa, Artificial Sequence
  Description of Artificial Sequence: Synthetic peptide
VQYNYLQR

SEQ ID NO: 275 - protein, 8 aa, Artificial Sequence
  Description of Artificial Sequence: Synthetic peptide
NSLLKHLL

SEQ ID NO: 276 - protein, 8 aa, Artificial Sequence
  Description of Artificial Sequence: Synthetic peptide
AIRLHLMG

SEQ ID NO: 277 - protein, 8 aa, Artificial Sequence
  Description of Artificial Sequence: Synthetic peptide
NSLRKHLL

SEQ ID NO: 278 - protein, 8 aa, Artificial Sequence
  Description of Artificial Sequence: Synthetic peptide
NIKLFANN

SEQ ID NO: 279 - protein, 8 aa, Artificial Sequence
  Description of Artificial Sequence: Synthetic peptide
SLLFHLLK

SEQ ID NO: 280 - protein, 8 aa, Artificial Sequence
  Description of Artificial Sequence: Synthetic peptide
VLLHYGLY

SEQ ID NO: 281 - protein, 8 aa, Artificial Sequence
  Description of Artificial Sequence: Synthetic peptide
AIRGALMG

SEQ ID NO: 282 - protein, 8 aa, Artificial Sequence
  Description of Artificial Sequence: Synthetic peptide
FYNMYLLN

SEQ ID NO: 283 - protein, 8 aa, Artificial Sequence
  Description of Artificial Sequence: Synthetic peptide
LLHSSKSQ

SEQ ID NO: 284 - protein, 8 aa, Artificial Sequence
  Description of Artificial Sequence: Synthetic peptide
LLHKSKSQ

SEQ ID NO: 285 - protein, 8 aa, Artificial Sequence
  Description of Artificial Sequence: Synthetic peptide
ANNLRLLH

SEQ ID NO: 286 - protein, 8 aa, Artificial Sequence
  Description of Artificial Sequence: Synthetic peptide
ANNSRLLH

SEQ ID NO: 287 - protein, 8 aa, Artificial Sequence
  Description of Artificial Sequence: Synthetic peptide
AANMFLLL

SEQ ID NO: 288 - protein, 8 aa, Artificial Sequence
  Description of Artificial Sequence: Synthetic peptide
LYLLKHHP

SEQ ID NO: 289 - protein, 8 aa, Artificial Sequence
  Description of Artificial Sequence: Synthetic peptide
NNSWRLHL

SEQ ID NO: 290 - protein, 8 aa, Artificial Sequence
  Description of Artificial Sequence: Synthetic peptide
ANNVRLLH

SEQ ID NO: 291 - protein, 346 aa, Unknown
  Description of Unknown: Annexin I polypeptide
MAMVSEFLKQAWFIDNEEQEYIKTVKGSKGGPGSAVSPYPTFNPSSDVEALHKAITVKGVDEATIIEILTKRTNAQRQQIKAAYLQEKGKPLDEALKKALTGHLEEVALALLKTPAQFDADELRAAMKGLGTDEDTLNEILASRTNREIREINRVYKEELKRDLAKDITSDTSGDYQKALLSLAKGDRSEDLAINDDLADTDARALYEAGERRKGTDLNVFITILTTRSYPHLRRVFQKYSKYSKHDMNKVLDLELKGDIENCLTVVVKCATSKPMFFAEKLHQAMKGIGTRHKTLIRIMVSRSEIDMNDIKACYQKLYGISLCQAILDETKGDYEKILVALCGGD

SEQ ID NO: 292 - protein, 320 aa, Unknown
  Description of Unknown: Annexin V polypeptide
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 293 - protein, 16 aa, Artificial Sequence
  Description of Artificial Sequence: Synthetic peptide
MEKEFPGFLENQKDPL

SEQ ID NO: 294 - protein, 10 aa, Artificial Sequence
  Description of Artificial Sequence: Synthetic peptide
PPAETELQAM

SEQ ID NO: 295 - protein, 11 aa, Artificial Sequence
  Description of Artificial Sequence: Synthetic peptide
LPSAEVELQAK

SEQ ID NO: 296 - protein, 9 aa, Artificial Sequence
  Description of Artificial Sequence: Synthetic peptide
  MOD_RES (7)..(9): Any amino acid
PGAYPGXXX

SEQ ID NO: 297 - protein, 15 aa, Artificial Sequence
  Description of Artificial Sequence: Synthetic peptide
LAIACHDSFIKSTQK

SEQ ID NO: 298 - protein, 19 aa, Artificial Sequence
  Description of Artificial Sequence: Synthetic peptide
LTIACNDYFVVHMKQKGKK

SEQ ID NO: 299 - protein, 7 aa, Artificial Sequence
  Description of Artificial Sequence: Synthetic peptide
MASPLDQ

SEQ ID NO: 300 - protein, 10 aa, Artificial Sequence
  Description of Artificial Sequence: Synthetic peptide
MASPLDQAIG

SEQ ID NO: 301 - protein, 11 aa, Artificial Sequence
  Description of Artificial Sequence: Synthetic peptide
WQQYEPNGKGT

SEQ ID NO: 302 - protein, 11 aa, Artificial Sequence
  Description of Artificial Sequence: Synthetic peptide
WKKDEPNDHGS

SEQ ID NO: 303 - protein, 13 aa, Artificial Sequence
  Description of Artificial Sequence: Synthetic peptide
AEVKKPVEAAAPG

SEQ ID NO: 304 - protein, 14 aa, Rattus sp.
VRSLSNTANVTWKL

SEQ ID NO: 305 - protein, 11 aa, Rattus sp.
FLVRDNQSGSL

SEQ ID NO: 306 - protein, 19 aa, Artificial Sequence
  Description of Artificial Sequence: Synthetic peptide
QMAGGFDEKAGGAQLGVMQ

SEQ ID NO: 307 - protein, 19 aa, Artificial Sequence
  Description of Artificial Sequence: Synthetic peptide
ALADEREAVQKKTFTKWVN

SEQ ID NO: 308 - protein, 12 aa, Artificial Sequence
  Description of Artificial Sequence: Synthetic peptide
YLSEKKGGNGKN

SEQ ID NO: 309 - protein, 24 aa, Artificial Sequence
  Description of Artificial Sequence: Synthetic peptide
DEAAGNLRQHDAEVDATLKSLNNQ

SEQ ID NO: 310 - protein, 14 aa, Artificial Sequence
  Description of Artificial Sequence: Synthetic peptide
FQSERSRARYEMAS

SEQ ID NO: 311 - protein, 12 aa, Artificial Sequence
  Description of Artificial Sequence: Synthetic peptide
YFVVHMKQKGKK

SEQ ID NO: 312 - protein, 12 aa, Artificial Sequence
  Description of Artificial Sequence: Synthetic peptide
YLSEKKGGNGKN

SEQ ID NO: 313 - protein, 14 aa, Artificial Sequence
  Description of Artificial Sequence: Synthetic peptide
NSIAYLDEAAGNLK

SEQ ID NO: 314 - protein, 11 aa, Artificial Sequence
  Description of Artificial Sequence: Synthetic peptide
SGDYWIDPNQG

SEQ ID NO: 315 - protein, 15 aa, Artificial Sequence
  Description of Artificial Sequence: Synthetic peptide
KEATSTFTNITYRGT

SEQ ID NO: 316 - protein, 8 aa, Artificial Sequence
  Description of Artificial Sequence: Synthetic peptide
AYLHYAAG

SEQ ID NO: 317 - protein, 8 aa, Artificial Sequence
  Description of Artificial Sequence: Synthetic peptide
AYLGYAAG

SEQ ID NO: 318 - protein, 8 aa, Artificial Sequence
  Description of Artificial Sequence: Synthetic peptide
AYLGHAAG

SEQ ID NO: 319 - protein, 8 aa, Artificial Sequence
  Description of Artificial Sequence: Synthetic peptide
AYLMHAAG

SEQ ID NO: 320 - protein, 8 aa, Artificial Sequence
  Description of Artificial Sequence: Synthetic peptide
AYLHHAAG

SEQ ID NO: 321 - protein, 8 aa, Artificial Sequence
  Description of Artificial Sequence: Synthetic peptide
AYLVHAAG

SEQ ID NO: 322 - protein, 8 aa, Artificial Sequence
  Description of Artificial Sequence: Synthetic peptide
AYLHGAAG

SEQ ID NO: 323 - protein, 8 aa, Artificial Sequence
  Description of Artificial Sequence: Synthetic peptide
SERYTARY

SEQ ID NO: 324 - protein, 8 aa, Artificial Sequence
  Description of Artificial Sequence: Synthetic peptide
SERYSARY

SEQ ID NO: 325 - protein, 8 aa, Artificial Sequence
  Description of Artificial Sequence: Synthetic peptide
SERYWARY

SEQ ID NO: 326 - protein, 8 aa, Artificial Sequence
  Description of Artificial Sequence: Synthetic peptide
SERCWARY

SEQ ID NO: 327 - protein, 8 aa, Artificial Sequence
  Description of Artificial Sequence: Synthetic peptide
SERCYARY

SEQ ID NO: 328 - protein, 8 aa, Artificial Sequence
  Description of Artificial Sequence: Synthetic peptide
SERYYARY

SEQ ID NO: 329 - protein, 8 aa, Artificial Sequence
  Description of Artificial Sequence: Synthetic peptide
SERFFARY

SEQ ID NO: 330 - protein, 8 aa, Artificial Sequence
  Description of Artificial Sequence: Synthetic peptide
  MOD_RES (4)..(5): Any amino acid
YLDXXAGN

SEQ ID NO: 331 - protein, 8 aa, Artificial Sequence
  Description of Artificial Sequence: Synthetic peptide
  MOD_RES (4)..(5): Any amino acid
SERXXARY

SEQ ID NO: 332 - protein, 5 aa, Artificial Sequence
  Description of Artificial Sequence: Synthetic peptide
  N-term H
LEQLL

SEQ ID NO: 333 - protein, 5 aa, Artificial Sequence
  Description of Artificial Sequence: Synthetic peptide
  N-term Mannosylacetyl
LEQLL

SEQ ID NO: 334 - protein, 9 aa, Artificial Sequence
  Description of Artificial Sequence: Synthetic peptide
  N-term H
  C-term NH2
KKILHRLLQ

SEQ ID NO: 335 - protein, 9 aa, Artificial Sequence
  Description of Artificial Sequence: Synthetic peptide
  N-term H
  misc_feature (2)..(6): Linkage between residues
  C-term NH2
  see specification as filed for detailed description of substitutions and preferred embodiments
KEILRKLLQ

SEQ ID NO: 336 - protein, 9 aa, Artificial Sequence
  Description of Artificial Sequence: Synthetic peptide
  N-term H
  MOD_RES (2)..(2): D-Cys
  misc_feature (2)..(5): Disulfide linkage between residues
  C-term NH2
KCILCRLLQ

SEQ ID NO: 337 - protein, 10 aa, Artificial Sequence
  Description of Artificial Sequence: Synthetic peptide
  N-term Ac
  MOD_RES (2)..(2): D-Cys
  misc_feature (2)..(5): Disulfide linkage between residues
  C-term NH2
KCILCRLLLQ

SEQ ID NO: 338 - protein, 10 aa, Artificial Sequence
  Description of Artificial Sequence: Synthetic peptide
  MOD_RES (1)..(1): Aib
  MOD_RES (3)..(3): D-Cys
  misc_feature (3)..(6): Disulfide linkage between residues
  C-term NH2
XKCILCRLLQ

SEQ ID NO: 339 - protein, 7 aa, Artificial Sequence
  Description of Artificial Sequence: Synthetic peptide
  MOD_RES (4)..(5): Any amino acid
PCFXXLP

SEQ ID NO: 340 - protein, 7 aa, Artificial Sequence
  Description of Artificial Sequence: Synthetic peptide
SDPPSPS

SEQ ID NO: 341 - protein, 4 aa, Artificial Sequence
  Description of Artificial Sequence: Synthetic peptide
CGSG

SEQ ID NO: 342 - protein, 16 aa, Artificial Sequence
  Description of Artificial Sequence: Synthetic peptide
VRSLSNSTARNVTWKL

SEQ ID NO: 343 - protein, 10 aa, Artificial Sequence
  Description of Artificial Sequence: Synthetic peptide
YLEGLLMHQA

SEQ ID NO: 344 - protein, 8 aa, Artificial Sequence
  Description of Artificial Sequence: Synthetic peptide
LLASLLQS

SEQ ID NO: 345 - protein, 9 aa, Artificial Sequence
  Description of Artificial Sequence: Synthetic peptide
HLKTLLKKS

SEQ ID NO: 346 - protein, 8 aa, Artificial Sequence
  Description of Artificial Sequence: Synthetic peptide
QLALLLSS

SEQ ID NO: 347 - protein, 9 aa, Artificial Sequence
  Description of Artificial Sequence: Synthetic peptide
LLLHLLKSQ

SEQ ID NO: 348 - protein, 9 aa, Artificial Sequence
  Description of Artificial Sequence: Synthetic peptide
VTLLQLLLG

SEQ ID NO: 349 - protein, 8 aa, Artificial Sequence
  Description of Artificial Sequence: Synthetic peptide
VLQLLLGN

SEQ ID NO: 350 - protein, 8 aa, Artificial Sequence
  Description of Artificial Sequence: Synthetic peptide
LLSRLLRQ

SEQ ID NO: 351 - protein, 10 aa, Artificial Sequence
  Description of Artificial Sequence: Synthetic peptide
VLKQLLLSEN

SEQ ID NO: 352 - protein, 8 aa, Artificial Sequence
  Description of Artificial Sequence: Synthetic peptide
VLKQLLLS

SEQ ID NO: 353 - protein, 9 aa, Artificial Sequence
  Description of Artificial Sequence: Synthetic peptide
ELAELLSAN

SEQ ID NO: 354 - protein, 8 aa, Artificial Sequence
  Description of Artificial Sequence: Synthetic peptide
SLGPLLLE

SEQ ID NO: 355 - protein, 9 aa, Artificial Sequence
  Description of Artificial Sequence: Synthetic peptide
KLVQLLTTT

SEQ ID NO: 356 - protein, 8 aa, Artificial Sequence
  Description of Artificial Sequence: Synthetic peptide
ILHRLLQE

SEQ ID NO: 357 - protein, 8 aa, Artificial Sequence
  Description of Artificial Sequence: Synthetic peptide
LLRYLLDK

SEQ ID NO: 358 - protein, 9 aa, Artificial Sequence
  Description of Artificial Sequence: Synthetic peptide
QLDELLCPP

SEQ ID NO: 359 - protein, 8 aa, Artificial Sequence
  Description of Artificial Sequence: Synthetic peptide
LLQQLLTE

SEQ ID NO: 360 - protein, 8 aa, Artificial Sequence
  Description of Artificial Sequence: Synthetic peptide
QLSELLRG

SEQ ID NO: 361 - protein, 11 aa, Artificial Sequence
  Description of Artificial Sequence: Synthetic peptide
YLEGLLMHQAA

SEQ ID NO: 362 - protein, 11 aa, Artificial Sequence
  Description of Artificial Sequence: Synthetic peptide
LLASLLQSESS

SEQ ID NO: 363 - protein, 11 aa, Artificial Sequence
  Description of Artificial Sequence: Synthetic peptide
HLKTLLKKSKV

SEQ ID NO: 364 - protein, 11 aa, Artificial Sequence
  Description of Artificial Sequence: Synthetic peptide
QLALLLSSEAH

SEQ ID NO: 365 - protein, 11 aa, Artificial Sequence
  Description of Artificial Sequence: Synthetic peptide
LLLHLLKSQTI

SEQ ID NO: 366 - protein, 11 aa, Artificial Sequence
  Description of Artificial Sequence: Synthetic peptide
LLQLLLGHKNE

SEQ ID NO: 367 - protein, 11 aa, Artificial Sequence
  Description of Artificial Sequence: Synthetic peptide
VLQLLLGNPKG

SEQ ID NO: 368 - protein, 11 aa, Artificial Sequence
  Description of Artificial Sequence: Synthetic peptide
LLSRLLRQNQD

SEQ ID NO: 369 - protein, 11 aa, Artificial Sequence
  Description of Artificial Sequence: Synthetic peptide
VLKQLLLSENC

SEQ ID NO: 370 - protein, 11 aa, Artificial Sequence
  Description of Artificial Sequence: Synthetic peptide
KLVQLLTTTAE

SEQ ID NO: 371 - protein, 11 aa, Artificial Sequence
  Description of Artificial Sequence: Synthetic peptide
ILHRLLQEGSP

SEQ ID NO: 372 - protein, 11 aa, Artificial Sequence
  Description of Artificial Sequence: Synthetic peptide
LLRYLLDKDEK

SEQ ID NO: 373 - protein, 8 aa, Artificial Sequence
  Description of Artificial Sequence: Synthetic peptide
LLQQLLTE

SEQ ID NO: 374 - protein, 11 aa, Artificial Sequence
  Description of Artificial Sequence: Synthetic peptide
KLLQLLTTKSD

SEQ ID NO: 375 - protein, 11 aa, Artificial Sequence
  Description of Artificial Sequence: Synthetic peptide
ILHRLLQDSSS

SEQ ID NO: 376 - protein, 11 aa, Artificial Sequence
  Description of Artificial Sequence: Synthetic peptide
LLRYLLDKDDT

SEQ ID NO: 377 - protein, 11 aa, Artificial Sequence
  Description of Artificial Sequence: Synthetic peptide
QLSELLRGGSG

SEQ ID NO: 378 - protein, 11 aa, Artificial Sequence
  Description of Artificial Sequence: Synthetic peptide
QLVLLLHAHKC

SEQ ID NO: 379 - protein, 11 aa, Artificial Sequence
  Description of Artificial Sequence: Synthetic peptide
QLSELLRGSSP

SEQ ID NO: 380 - protein, 11 aa, Artificial Sequence
  Description of Artificial Sequence: Synthetic peptide
QLVLLLHAHKC

SEQ ID NO: 381 - protein, 11 aa, Artificial Sequence
  Description of Artificial Sequence: Synthetic peptide
ILTSLLLNSSQ

SEQ ID NO: 382 - protein, 11 aa, Artificial Sequence
  Description of Artificial Sequence: Synthetic peptide
MLMNLLKDNPA

SEQ ID NO: 383 - protein, 11 aa, Artificial Sequence
  Description of Artificial Sequence: Synthetic peptide
TLRSLLLNPHL

SEQ ID NO: 384 - protein, 11 aa, Artificial Sequence
  Description of Artificial Sequence: Synthetic peptide
RLAVLLPGRHP

SEQ ID NO: 385 - protein, 11 aa, Artificial Sequence
  Description of Artificial Sequence: Synthetic peptide
ELHNLLEVVSQ

SEQ ID NO: 386 - protein, 11 aa, Artificial Sequence
  Description of Artificial Sequence: Synthetic peptide
TLRDLLTTTAG

SEQ ID NO: 387 - protein, 11 aa, Artificial Sequence
  Description of Artificial Sequence: Synthetic peptide
FLDFLLGFSAG

SEQ ID NO: 388 - protein, 11 aa, Artificial Sequence
  Description of Artificial Sequence: Synthetic peptide
VLELLLRAGAN

SEQ ID NO: 389 - protein, 11 aa, Artificial Sequence
  Description of Artificial Sequence: Synthetic peptide
ILARLLRAHGA

(19) United States
(12) Patent Application Publication
(10) Pub. No.: US 2013/0053541 A1
(43) Pub. Date: Feb. 28, 2013

(54) METHODS FOR DISCOVERING MOLECULES THAT BIND TO PROTEINS

(71) Applicants: Sriram Shankar, College Station, TX (US); Harold Ray Garner, JR., Blacksburg, VA (US); Richard McAloney, Toronto (CA); Duncan Hitchens, Allen, TX (US)

(72) Inventors: Sriram Shankar, College Station, TX (US); Harold Ray Garner, JR., Blacksburg, VA (US); Richard McAloney, Toronto (CA); Duncan Hitchens, Allen, TX (US)

(73) Assignee: LYNNTECH, INC., College Station, TX (US)

(21) Appl. No.: 13/416,607

(22) Filed: Mar. 9, 2012

Related U.S. Application Data
(60) Provisional application No. 61/452,025, filed on Mar. 11, 2011.

(51) Int. Cl. C40B 30/04; C07K 2/00

(52) U.S. Cl. 530/300; 506/9

(57) ABSTRACT

Methods and systems for the discovery of high-affinity peptide ligands and the resulting compositions are described herein. The amino acid sequence of a target protein is used to identify one or more homologous proteins of the target protein. Publications and databases are textmined to retrieve the sequences of peptide ligands that bind to the homologues or the target protein. Complementary proteins, which are proteins that bind to the target or homologous proteins or to DNA, and their target protein- or DNA-binding regions may also be identified. These candidate ligands are predicted to have a high probability of binding to the target protein or the DNA. The library of candidate peptide ligands is modulated by substituting native amino acid residues with suitable amino acids, thus increasing the explored protein space in a knowledge-based manner. Peptides designed in the modulation step are experimentally screened to identify high-affinity binding ligands, and further optimized through iterative application of the modulation and screening steps.

### CROSS-REFERENCE TO RELATED APPLICATIONS

This application is a non-provisional application of U.S. Provisional Patent Application Ser. No. 61/452,025 filed on Mar. 11, 2011 and entitled “METHODS FOR DISCOVERING MOLECULES THAT BIND TO PROTEINS” the entire contents of which is incorporated herein by reference.

### TECHNICAL FIELD OF THE INVENTION

The present invention relates in general to the field of methods of identifying peptides and peptide-like molecules with desirable properties (including affinity, specificity, selectivity, in vitro and in vivo availability and viability, and others), and more particularly, to novel, short peptides or peptide-like molecules which have a high probability of binding to and/or otherwise modulating the function of polypeptides, proteins, or DNA, and to methods for designing and validating the function of such peptides or peptide-like molecules.

### REFERENCE TO A SEQUENCE LISTING

The instant application contains a Sequence Listing which has been submitted in ASCII format via EFS-Web and is hereby incorporated by reference in its entirety. Said ASCII copy, created on May 11, 2012, is named LYNN1000.txt and is 107,967 bytes in size.

### BACKGROUND OF THE INVENTION

Without limiting the scope of the invention, its background is described in connection with methods for identifying and selecting peptides or peptide-like molecules capable of binding to and/or otherwise modulating the function of cells and their components.

Future identification and understanding of disease etiology, progression, therapy choice, and therapy response will require the ability to sensitively monitor vast panels (10 s-100 s) of protein biomarkers in parallel. Significant progress in proteome characterization has been made possible through technological advancements in protein production methods, mass spectrometry, and microfluidics and other protein separation methods. High affinity probe molecules able to specifically recognize biomarker proteins present at low concentrations in complex biological samples are critically important and desirable.

Traditionally, antibodies have satisfied the demand for high affinity ligands. However, their production and purification is time intensive and laborious. As information continues to accrue at a rapid rate, concerning both the composition of an organism's proteome and sequence information of new target proteins, rapid methods to generate affinity reagents are needed urgently in order to quickly identify, validate, and assay these new biomarker proteins. Likewise, methods offering the possibility of rapidly generating multiple high-affinity reagents against large numbers of proteins in the proteome are also desired.

Short high-affinity peptides (˜8-15 amino acids) are attractive as ligand alternatives to antibodies. They can be made in large quantities, and purified much more cheaply and easily. Therefore, they are preferable over biologically produced molecules. They have been used extensively in high throughput protein identification techniques that involve random generation of large combinatorial peptide libraries. However, the drawback to these methods is the time and cost required to generate and to screen these vast libraries, given the enormous diversity of ligand chemistry, and the fact that no rational design is involved. Consequently, there is a substantial need for automated methods that can rapidly select and optimize high-affinity peptide ligands with highly selective binding properties.

Drug discovery is a protracted and expensive process, from hit-to-lead discovery to preclinical and clinical testing and FDA approval. The estimated time and cost for a single drug to make it to market, including developmental costs of all failed drugs, is approximately 12-15 years and $600-800 million. Protein-protein and protein-DNA interactions are fundamental to all biological functions. These biomolecular interactions play a key role in signal transduction and in regulating several cellular processes such as cell growth, differentiation, migration, adhesion, and cell death. It is understood that dysregulation of certain key protein interactions is central to the pathology of most diseases (e.g. p53-Mdm2, Bcl-X_{L}-Bax, αᵥβ₃ Integrin-MMP2 in cancer). Thus, modulating protein-protein and protein-DNA interactions has the promise of obtaining many novel therapeutic agents. In fact, the identification of small molecule, peptide, and peptidomimetic ligands that bind to proteins with high affinity and mediate their activity by affecting their complementary protein interactions has been the central motif of drug discovery approaches. The short high-affinity peptide ligands described hereinabove can also be used as selective markers (perhaps even against a particular protein disease modification), as a detection and diagnostic agent for a given protein or protein variant for disease diagnosis, forensics, etc.

There are many approaches that have been taken to identify drugs that disrupt key protein mediated process. In general, once a drug target has been identified, the protein is over-expressed, isolated, and purified, and used for high-throughput screening via in vitro assays; i.e. vast unfocussed libraries of compounds (including peptides) are screened against the target to find a lead molecule that, for example, inhibits or modulates the function of the target protein. Several lead compounds are chosen and lead optimization then proceeds to complete the preclinical program, from which downselected candidates are carried into clinical trials. The most inefficient step in this process is the lead optimization stage, where less than 1 in 5000 candidates is found suitable for clinical investigation.

So called fragment based design (FBD) screening strategies are also being used, where interactions of small drug-like fragments with the target protein are evaluated in high throughput, and selected hits are combined into a single, potent pharmacore. Although the FBD approach samples a more diverse ligand space of smaller molecules than does screening synthetic compound libraries, and has produced successful hit-to-lead candidates, the methodology is still based on generating hits from an unfocussed ligand space. A focused library of privileged structures, which would result from the present invention, could enhance the value and potency of this method for discovering new drugs.

Alternatively, structure based design is carried out. NMR or X-ray crystallography has been used to predict the molecular structure of the target protein. Both these analytical techniques also have been applied to high-throughput screening, vis-à-vis saturation-transfer NMR and high-throughput X-ray crystallography, to select candidates that perturb the protein active site. Despite advanced robotic techniques and modernization, this process is cumbersome. Finally, in silico methods are available for de novo prediction of the tertiary structures of proteins, when crystal structures are not available. Computational programs can carry out subsequent in silico docking studies that can predict compounds that would bind to a given protein structure. Owing to the complex nature of proteins and their flexible solution based structures, these methods are complex, and perform better when used for structure-based analog design from inhibitors identified initially by high-throughput screening.

In short, there are many approaches where the chemical and structural information of proteins has been described in detail but the principles and information cannot be easily applied to how to predict ligands to specific proteins.

Among the most successful approaches to finding ligands for modulating protein-protein interactions is by developing mimics of the interface amino acid residues for one of the binding partners. Several important cell signaling events are mediated by short unstructured peptide interactions with proteins (Pawson and Scott 1997). Because of the potent and specific effects of peptides on physiological responses of human and other species, peptides are of great interest as drug candidates. Since peptides typically interact with other peptides and proteins to produce their biological effects, there is a great deal of interest in being able to predict the interactions between a peptide and another protein. As a result, there is great interest in developing methods to predict sequences of peptides that will interact with a polypeptide protein target and produce a desired response.

There are several arguments to be made that lend merit to peptide-based drug design. Peptides are considered prototypical among so-called ‘privileged structures’ (which include oligonucleotides and bioactive natural compounds). Peptide molecules offer an incredible diversity not commonly found in small molecule libraries. Compared to conventional biomolecular drug compounds (e.g., antibodies, scFv), peptides have the benefits of being robust and easy to synthesize and purify in large quantities, and they are non-immunogenic (Dutton 2007). Theoretically, short peptide sequences of 10-20 amino acids can be selected to interact with DNA and virtually to any binding cleft on a protein, making them excellent leads for antagonist development. These clefts may be located at the active site where the peptide binding may disrupt or inhibit protein interaction. Alternatively, the peptide may modulate protein-protein interactions through allosteric binding effects (Allegretti, Bertini et al. 2008). Multiple DNA-binding proteins are known to be key players in transcriptional and epigenetic signaling, and short, high-affinity peptide based molecules are key leads for binding and modulating critical gene functions. Since peptide binding affinities to proteins can span a considerable range (μM to sub-nM), the response to binding may vary considerably, from perturbation to complete inhibition, which offers latitude in their choice for specific applications (including as affinity molecules for high-throughput biomarker validation and assays).

Undesirable biological properties of peptide drugs include their limited bioavailability and stability owing to rapid proteolytic degradation and elimination from circulation. However, several strategies are available to circumvent these limitations, including peptidomimetic development (Witt, Gillespie et al. 2001; Adessi and Soto 2002; Sillerud and Larson 2005; Vagner, Qu et al. 2008), development of pro-drugs, and the use of advanced drug delivery systems (micelles, vesicles, nanoparticles, etc.).

Peptides as drug leads have been applied successfully to search for antagonists of cell-surface receptors. Numerous publications have demonstrated the ability of peptides to specifically inhibit or activate protein or enzyme actions (Norman, Smith et al. 1999; Tao, Wendler et al. 2000; Christensen, Gottlin et al. 2001; Pini, Giuliani et al. 2005; Allegretti, Bertini et al. 2008). Several peptide agents that bind strongly to disease related proteins have already been identified; e.g., bombesin, octreotide, cRGD peptides (Sillerud and Larson 2005). Peptides now represent one of the fastest growing classes of new drugs. Although they currently account for only 2% of drugs on the market, they reportedly comprise roughly 50% of drugs in the pipelines of major drug manufacturers. The market for peptide drugs is now growing at a compound annual rate of 7.5% and is estimated to be worth in excess of $13 billion by 2010.

There is infinite diversity in protein structure and function. Thus, the process of finding selective, high-affinity peptide (or any) ligands is not trivial. Tested approaches to producing peptide ligands to protein targets include phage display and combinatorial peptide library screening. Combinatorial peptide library synthesis is tedious and expensive to set up, the represented ligand sample space is random and not comprehensive, and the binding ligand needs to be isolated from beads for structure determination by Edman degradation. Iterative affinity refinement is not possible. This tedious screening approach has become less common.

Phage display has demonstrated the greatest success in peptide ligand discovery for proteins (Dias, Fasan et al. 2006). However, this method has certain key limitations, critical among which is that the random libraries represent only fractional diversity of the total ligand space; e.g., an 8-mer peptide can have 20⁸≈26 billion permutations, only ˜10% of which are expressed and screened. Furthermore, affinities are often only modest (μM range), and iterative affinity refinement by systematically modifying the peptide sequence is not feasible.

The present invention makes it possible to predict and test peptide molecules that will bind specifically and with high affinity to a desired protein (or DNA) without significant or substantial knowledge of the structure or function of protein being targeted other than its primary linear amino acid sequence. In this respect it is distinct from the processes used in general for drug molecule discovery, which often requires knowledge of the target beyond the linear sequence. It will be understood by persons skilled in the art that though the primary focus is on identifying peptide molecules for possible uses in therapy, there are many additional uses for the peptide with specific binding properties identified by this invention. Notably, such molecules can be used in diagnostics as molecular probes as well as in drug delivery and drug discovery (e.g., in vitro assays to screen against vast drug libraries). Furthermore, the present invention can be used for the identification of selective, high-affinity peptide binding agents, which can be used in molecular imaging, pathogen or rare cell capture, protein purification through affinity chromatography, forensics, etc.

### SUMMARY OF THE INVENTION

The present invention relates to methods and computer systems (HALO/Gen; High Affinity Ligand Optimization/Generation) for identifying and selecting peptides or peptide-like molecules capable of binding to, detecting, and/or otherwise modulating the function of protein targets having known amino acid sequences. Furthermore, the invention relates to compositions comprising the peptides identified and to methods to optimize their physiochemical properties and biological activity.

The present invention includes a method for discovery of high-affinity peptide ligands which comprises the steps of (i) obtaining an amino acid sequence of at least a portion of a target protein; (ii) identifying one or more sequence homologous proteins (with 20%, 30%, 40%, 50%, 60%, 70%, 80%, 90% and 100% homology) of the target protein; (iii) identifying complementary proteins, which are proteins that bind to/interact with the homologous proteins; (iv) extracting from databases peptides that have a ligand binding probability for the target and homologous proteins; (v) generating a library of candidate peptide ligands and their knowledge based sequence permutations; (vi) determining if any of the candidate peptide ligands binds to the target protein; and (vii) iteratively optimizing affinity and specificity for the target protein. The method may further comprise one or more “sequence modulation” steps, wherein sub-sequences of the candidate peptide ligand sequence, each one corresponding to an n-residue long contiguous sequence, are selected to yield a super-library of n-mer parent peptides (the “n-mer superlibrary”); and, one or more residues in each peptide in the n-mer superlibrary are substituted with other amino acids, to yield a “substituted n-mer superlibrary.”

Peptides of 8-12 amino acids may be selected for the n-mer superlibrary. The peptides used in the sequence modulation step are often octamers (i.e., n=8). It will be understood by the skilled artisan that the absolute length need is not limiting and is dictated by a number of factors, for e.g. specificity, sensitivity, ease of manufacture and purification, length constraints to enter cells, etc. Generally, two residues are substituted during sequence modulation, preferably located in central positions in the peptide. In another embodiment, the central four or six residues are permuted. However, it is possible to keep the central position in the peptide unchanged (e.g., positions 3, 4, 5, and/or 6 in an octamer), whereas pairs or other combinations of residues in peripheral positions are substituted and permuted. For example, residues 1, 2, 7, and/or 8 could be substituted. In another embodiment of the present invention, peptides could be scanned positionally such that one residue in each peptide in the n-mer superlibrary was substituted sequentially by a scanning amino acid, such as alanine.

The amino acid substitutions may be randomly selected, or they may be knowledge-based. A possible type of knowledge-based amino acid substitution is based on the use of a substitution matrix. A substitution matrix belonging to the BLOSUM (BLOcks of Amino Acid SUbstitution Matrix) family of substitution matrices may be used. An artisan will appreciate that there are numerous substitution matrices available, based on amino acid properties such as mutational frequency, physicochemical similarities, tendency to occupy a certain location on a protein tridimensional structure, tendency to covary, or tendency to adopt a certain secondary structure.

The present invention may further comprise the step of “screening” peptides selected from the library of candidate peptide ligands, the n-mer superlibrary, and the substituted n-mer superlibrary for their ability to bind to the target protein using laboratory methods. This screening step and the sequence modulation step may be repeated iteratively to identify the amino acids that contribute to optimal binding (defined as greatest affinity and specificity or selective binding or modulating ability) of peptides selected from the library of candidate peptide ligands, the n-mer superlibrary, and the substituted n-mer superlibrary to the target protein. A collection of peptides selected from the library of candidate peptide ligands, the n-mer superlibrary, and the substituted n-mer superlibrary may be screened using high-throughput peptide microarrays. In one embodiment of the present invention, the collection of peptides to be screened may be synthesized on the microarray chip substrate using a digitally controlled light source. In another embodiment, peptides may be synthesized and spotted on a cellulose or other matrix. The final result of this process is selecting peptide ligand sequences having highest binding affinity and specificity for, or most desirable biological activity on the protein-protein interactions of, the query protein.

The skilled artisan will appreciate that a variety of screening methods may be used. Some non-limiting examples of the methods that may be used include radiometric and spectrophotometric methods, fluorescence based methods, e.g., FRET (Fluorescence Resonance Energy Transfer), FET, ligand displacement assays, NMR methods, microarray-based analysis, pep-spot analysis, combinatorial libraries, and phage display, although other techniques could also be used.

The screening of peptides selected from the initial iteration or a subsequent iteration of a library of candidate peptide ligands, its respective n-mer superlibrary, and its substituted n-mer superlibrary may include the steps of: (i) determining the binding affinity of ligands; (ii) determining in vitro the inhibitory ability of ligands; (iii) performing cell-based activity assays that are selected based on the cellular target(s), protein target(s), cellular activation, cellular proliferation, secretion or expression of proteins, genes, or the transcription of RNA; (iv) performing animal-model based pharmacodynamics/pharmacokinetics assays for ADME-Tox or other pharmacological properties; and/or (v) performing preclinical/clinical testing in patient cohorts.

The target protein or fragment thereof may be involved in peptide-protein, protein-protein, or protein-DNA interactions, and the effect of the peptide ligand sequences may be to bind to and otherwise modulate, activate, or inhibit the function of the target protein or homologous proteins. The target protein may also be a post-translationally modified protein or a non-natural protein, such as a recombinant protein, a synthetic protein, a hydrolysis product, or a peptidomimetic. The target protein may be selected from the group consisting of cell membrane receptors, nuclear membrane receptors, cellular and nuclear proteins, mitochondrial proteins, circulating peptide and non-peptide receptors, membrane and circulating transporters, enzymes, chaperonins and chaperonin-like proteins, antibodies, and surface and intracellular proteins of infectious agents.

In the present invention, the identification of one or more proteins homologous to the target protein is performed by querying one or more suitable databases with one or more suitable homology search tools, using the target protein primary sequence or a fragment thereof as input. The concept of homology is used to denote similarities between primary sequences, without implying that the proteins share a common ancestry. The sequence of the target protein can be retrieved from a public or private protein database. However, it is also possible to obtain the primary sequence of the target protein by protein sequencing, or by translating (i) the nucleic acid sequence of a newly sequenced biological target of interest, or (ii) a nucleic acid sequence deposited in a public or private database.

The significance of the detected sequence similarity (homology) may be judged by an alignment score below a threshold of statistical significance. Numerous tools may be used to identify sequence homology. One or more tools may be used to detect sequence homology. The homology search tools may use sequence-to-sequence comparison (pairwise sequence alignment), using an algorithm such as Smith-Waterman, Needleman-Wunsch, BLAST, PSI-BLAST, PHI-BLAST, WU-BLAST2, BLAT, or FASTA.

The homology search tools may also use sequence to profile comparison (sequence-profile method). A number of methods may be used, including Position Specific Scoring Matrix (PSSM)-based methods such as PSI-BLAST, Hidden Markov Model (HMM)-based methods such as HHMER or SAM, or profile to profile comparison (profile-profile) methods such as FFAS, ORFeus, COMPASS, COACH, or HHpred.

Homology search tools may include protein domain comparison tools such as RPS-BLAST, HMMER or IprScan. These protein domain comparison tools may be applied to search for homology between the target protein primary sequence or a fragment thereof and protein domains stored in an individual protein domain databases, such as Pfam, SMART, PROSITE, Propom, PRINTS, UniProt, TIGRFAMs, PIR-SuperFamily, or SUPERFAMILY. Furthermore, these domain comparison tools are applied to search for homology between the target protein primary sequence or a fragment thereof and protein domain stored in a protein domain meta-database, such as InterPro or CCD.

The homology search tools may include protein domain architecture comparison tools, such as CDART. The identification of peptides that have a ligand binding probability may also be performed using functional-domain-prediction bioinformatic methods such as ab-initio domain predictions, secondary structure predictions, disorder/low complexity predictions, linker predictions, gene fusion methods, protein functional domain co-occurrence methods, genetic context methods (e.g., gene neighborhoods, gene clusters, operons), phylogenomic profiles, and metabolic reconstructions.

The present invention may comprise the further step of identifying “complement proteins.” These “complement proteins” are proteins known to interact with the target protein (or to DNA), with proteins homologous to the target protein, and with fragments of the target protein or its homologues. The target protein, proteins homologous to the target protein, and/or fragments of the target protein or its homologues would be used to query one or more protein-protein interaction databases, such as DIP and IntAct, such that proteins known to interact with the query sequences would be identified. Complement proteins may include enzymes that degrade the target protein, proteins degraded by a target protein that is an enzyme, regulatory proteins, structural proteins, et cetera. The target protein might be also its own complement protein when the protein's quaternary structure is multimeric.

The identification of peptides with a probability of being ligands binding to the target protein or DNA may be performed using textmining. Candidate peptide ligands likely to bind to the target protein, candidate peptide ligands likely to bind to homologous proteins, and candidate peptide ligands present in the sequence of complement proteins that are likely to bind to the target protein, DNA, or to homologous proteins may be identified using textmining. Textmining may include mining and curating ligand-binding data collected from scientific literature, protein-ligand databases, publications, literature reports, documents, computerized records, abstracts, scientific journals, and other public and non-public sources (all these different types of publications are collectively described as “data sources”). Textmining may be manual, computer-assisted, or any combination thereof.

Candidate peptide ligands may be identified by text searching publicly accessible databases using customized search terms, for references including peptide ligands and interacting sequences or domains to the target protein and/or to homologous proteins. Textmining may be performed by querying “data sources” using one or more sequences or fragments of the target protein, homologs, or complement protein. Furthermore, the “data sources” may be queried with one or more protein names, synonyms, commonly accepted acronym, database accession numbers, domain names, E.C. numbers, or any other non-sequence suitable identifier. A publicly available database such as BioMint, UniProt, or Entrez may be employed as a source to obtain protein name synonyms and other related information about protein.

The textmining step may be a single step, or a group of textmining steps. The textmining space may be expanded by querying the “data sources” with permutations and combinations of search terms including sequences or fragments of the target protein name, and those of homologs, or complement proteins; protein names; synonyms; commonly accepted acronyms; database accession numbers; domain names; E.C. numbers; or other suitable identifiers. The search terms may include non-identifier keywords; for example sequence, ligand, peptide, epitope, paratope, motif, etc.

Once candidate peptides are identified, the candidate peptide ligands may be ranked according to the degree of sequence homology between the primary sequence of the homologous protein used for the textmining query and the primary sequence of the target protein, wherein ligands corresponding to proteins with higher homology scores would be ranked highest.

The library of candidate peptide ligand sequences may be culled of redundant or “entrained” sequences using one or more Multiple Sequence Alignment (MSA) algorithms, such that when two sequences are identical or almost identical, only the longer of the two sequences is selected. For example, MSA could be performed using ClustalW2, DCA, Dialign, POA, T-Coffee, MAFFT, or MUSCLE. The inventors also created two databases called LiBase and PepLib comprising other ligand binding databases, the text mining derived data and experimental data from array work done by the inventors. This database is particularly useful since it comprises experimental data that will become increasingly important and more accurate as its size increases with new ligand binding results from studies conducted by the inventors.

The present invention also includes a computer program embodied on a computer readable medium capable of performing the methods disclosed. Another embodiment of the present invention is a computer system, comprising programming to perform the method disclosed. The present invention also includes a computer storage media, comprising programming to perform the methods disclosed. Furthermore, the present invention includes the compositions produced by applying the methods disclosed.

In one embodiment, the instant invention provides a method for discovery of one or more high-affinity peptide ligands comprising: (i) obtaining an amino acid sequence of at least a portion of a target protein, (ii) identifying one or more homologous proteins of the target protein, (iii) identifying complementary proteins, wherein the complementary proteins are proteins that bind to the target or homologous proteins, (iv) extracting from literature or from one or more databases, peptides that have a ligand binding probability for the target protein or homologous proteins, (v) generating a library of one or more candidate peptide ligands from the extracted literature; and (vi) determining if any of the one or more candidate peptide ligands binds to the target protein. The target protein as described above is selected from the group consisting of cell membrane receptors, nuclear membrane receptors, cytoplasmic, nuclear, and mitochondrial proteins, secreted proteins, circulating peptide and non-peptide receptors, membrane and circulating transporters, enzymes, chaperonins and chaperonin-like proteins, antibodies, and surface and intracellular proteins of infectious agents.

The method as described hereinabove further comprises the steps of: performing a “sequence modulation,” wherein subsequences of the one or more candidate peptide ligand sequences each one corresponding to an n-residue long contiguous sequence are selected to yield a super-library of n-mer parent peptides (the “n-mer superlibrary”) and substituting one or more residues in each peptide in the n-mer superlibrary with other amino acids, to yield a “substituted n-mer superlibrary.” In one aspect of the method of the present invention, the one residue in each peptide in the n-mer superlibrary is sequentially substituted by a scanning amino acid (by alanine). The amino acid substitutions are knowledge-based and are selected using a substitution matrix, wherein the substitution matrix is a member of the BLOSUM (BLOcks of Amino Acid SUbstitution Matrix) family of substitution matrices, PAM or any custom generated matrix from data analysis. In another aspect of the method disclosed herein, n is 8. In yet another aspect, two, four, six, or all n residues are substituted. In another aspect, the two substituted residues occupy central positions in the peptide. In another aspect, one or more central amino acids of the peptide are kept unchanged while pairs of outlying amino acids in the sequence are permuted. In related aspects, the amino acid residues in positions 1 and 8 or in positions 2 and 7 are substituted and the substitutions are random variations of the peptide chemistry.

The method of the present invention further comprises the step of “screening” peptides selected from the library of candidate peptide ligands, the n-mer superlibrary, and the substituted n-mer superlibrary for binding ability to the target protein using one or more experimental methods. In one aspect of the method the steps of “sequence modulation” and “screening” iteratively to identify the amino acids which contribute to optimal binding of peptides selected from the library of candidate peptide ligands, the n-mer superlibrary, and the substituted n-mer superlibrary to the target protein, and thereby selecting candidate peptide ligand sequences having optimized binding activity (evaluated using an established assay method selected from the group consisting of FRET (Fluorescence Resonance Energy Transfer), spectrophotometric assay, fluorescence assay, NMR, MS, electrical/electronic (e.g., FET, SPR) assays, in silico docking, microarray-based analysis, pep-spot analysis, combinatorial libraries, phage display, cell based assays, and combinations or modifications thereof). In another aspect, the optimized binding activity comprises affinity (Kd), specificity, selectivity, in vitro and in vivo availability, viability, and combinations and modifications thereof. The affinity may be modulated by dimerization, oligomerization, multivalent display, or any combinations thereof to facilitate multiplex binding. In yet another aspect, a collection of peptides selected from the library of candidate peptide ligands, the n-mer superlibrary, and the substituted n-mer superlibrary is screened using high-throughput peptide microarrays selected from the group consisting of chip based assays, liquid arrays comprising flow cytometry bead assays, multiplex labeled beads on a substrate, or any combinations thereof. The collection of peptides is synthesized on the microarray chip substrate using a digitally controlled light source, in situ laser printing, PEPspot and other pre-synthesized peptide spotting technologies or any combinations thereof. In a specific aspect, about 4,000 to 2,000,000 peptides are arrayed on the chip.

In one aspect of the method of the present invention, the screening of peptides selected from an initial iteration or a subsequent iteration of a library of candidate peptide ligands, its respective n-mer superlibrary, and its substituted n-mer superlibrary includes: a) determining the binding affinity of ligands in vivo or in vitro by linking to a contrast agent or a detection agent, wherein the contrast agent or the detection agent is selected from the group consisting of a radioactive tracer, a GFP, a fluorophore, a quantum dot, a nanoscale structure or a nanowire, b) determining in vitro modulatory (e.g., agonist, inverse agonist, antagonist) ability of ligands towards one or more agonists, inverse agonists, or antagonists, and c) performing cell-based activity assays, in vivo pharmacological assays, drug sensitivity assays, clinical/preclinical testing, or any combinations thereof.

In another aspect, the target protein or fragment thereof is involved in, but not limited to, peptide-protein or protein-protein or protein-small molecule interactions. In yet another aspect, the peptide ligand sequences bind to and otherwise modulate, activate, or inhibit the function of the target protein, homologous proteins, or DNA. In another aspect, the peptide ligands sequences can be polyvalently linked or displayed for protein, DNA, and cell capture. In another aspect, peptide ligands can be conjugated to a payload such as a drug, protein or other molecules such as, but not limited to, liposomes, dendrimers, or combinations thereof for drug delivery. In yet another aspect, the step of identifying one or more homologous protein is performed using the target protein primary sequence or a fragment thereof to query one or more suitable databases with one or more suitable homology search tools, such that proteins homologous to the target protein are identified, wherein the query protein amino acid sequences are obtained by translating nucleic acid sequences. In one aspect of the present invention, the homologous sequences have significant sequence similarity to the target sequence as judged by an alignment score below a threshold of statistical significance. In another aspect, the one or more of the homology search tools includes sequence to sequence comparison (pairwise sequence alignment) done by an algorithm (selected from the group consisting of Smith-Waterman, Needleman-Wunsch, BLAST, PSI-BLAST, PHI-BLAST, WU-BLAST2, BLAT, and FASTA).

In yet another aspect of the method, the one or more of the homology search tools is based on sequence to profile comparison (sequence-profile method) method. In a specific aspect, the profile method is Position Specific Scoring Matrix (PSSM)-based (PSI-BLAST) or a Hidden Markov Model (HMM) based method (HHMER or SAM). In a related aspect, the one or more of the homology search tools is based on profile to profile comparison (profile-profile) method, wherein the profile-profile method is selected from the group consisting of FFAS, ORFeus, COMPASS, COACH, and HHpred. In another aspect, the one or more of the homology search tools includes protein domain comparison tools, wherein the domain comparison tools include CDD, CDART, RPS-BLAST, HMMER, or IprScan. In yet another aspect the domain comparison tools are applied to search for homology between the target protein primary sequence or a fragment thereof and protein domain stored in an individual protein domain databases (selected from the group consisting of Pfam, SMART, PROSITE, Propom, PRINTS, UniProt, TIGRFAMs, PIR-SuperFamily, and SUPERFAMILY) or a protein domain meta-database (InterPro, CDD or CDART). In another related aspect, one or more of the homology search tools includes protein domain architecture comparison tools, more specifically CDART.

In one aspect of the method disclosed herein, the identification of peptides that have a protein or DNA binding probability includes functional domain prediction bioinformatic methods from the group consisting of ab-initio domain predictors, secondary structure predictors, disorder predictors, linker predictors, gene fusion methods, domain co-occurrence methods, genetic context methods (gene neighborhoods, gene clusters and operons), phylogenomic profiles, and metabolic reconstruction. The method of the present invention further comprises the step of identifying “complement proteins” wherein sequences selected from group consisting of the target protein, the DNA, the homologous proteins, and fragments thereof are queried against one or more protein-protein interaction databases, such that proteins known to interact with the query sequences are identified. The protein-protein interaction databases that are queried include DIP IntAct and DOMINO, and all other open-source computational or manually curated databases.

In another aspect of the method disclosed herein, the identification of peptides with a ligand binding probability is performed using textmining, such that candidate peptide ligands are likely to bind to the target protein, candidate peptide ligands are likely to bind to the DNA, candidate peptide ligands are likely to bind to homologous proteins, and candidate peptide ligands present in the sequence of complement proteins that are likely to bind to the target protein or to homologous proteins are identified. The step of textmining, as disclosed herein, includes mining and curating ligand-binding data collected from scientific literature and protein-ligand databases, publications, literature reports, documents, computerized records, abstracts, scientific journals, and other public and non-public sources (“data sources”). The textmining can be performed manually or is computer-assisted in which case it is performed using one or more text similarity search engines, wherein the search engine comprises eTBLAST, Contaro, the search engine and database HALO, or any combinations or modifications thereof. It will be understood herein that the textmining is both manual and computer-assisted.

In yet another aspect of the method, the candidate peptide ligands are identified by text searching publicly accessible databases using customized search terms, for references including peptide ligands and interacting sequences, epitopes/paratopes, motifs, regions, and/or domains to the target protein and/or to homologous proteins, wherein input text used to search a database for relevant protein-ligand interactions in the protein synopsis is selected from the group consisting of, but not limited to, PubMed, UniProt, ChemAbstracts, PDB, or InterPro. In another aspect, the textmining is performed by querying “data sources” with one or more protein names, synonyms, commonly accepted acronyms, database accession numbers, domain names, E.C. numbers, or another non-sequence suitable identifiers of the target protein, homologs, or complement proteins. In a specific aspect, the BioMint, UniProt, PDB databases or combinations thereof are employed to obtain protein synonyms. In yet another aspect, the textmining is performed by querying “data sources” with permutations and combinations of search terms including sequences or fragments of the target protein, homologs, or complement proteins; protein names; synonyms; commonly accepted acronyms; database accession numbers; domain names; E.C. numbers; or other suitable identifiers, wherein the search terms include non-identifier keywords.

In another aspect of the method, the candidate peptide ligands are ranked according to the degree of sequence homology between the primary sequence of the homologous protein used for the textmining query and the primary sequence of the target protein, wherein ligands corresponding to proteins with higher homology scores are ranked highest. In yet another aspect, the evaluation of ligand binding probabilities is performed by querying a peptide database, wherein the peptide database is PepLib, LiBase or other databases. In one aspect of the method, the library of candidate peptide ligand sequences is culled of redundant or “entrained” sequence using a Multiple Sequence Alignment (MSA) algorithm (selected from the group consisting of ClustalW, DCA, Dialign, POA, T-Coffee, MAFFT, and MUSCLE), such that when two sequences are identical or almost identical, only the longest of the two sequences is selected. In a specific aspect, the MSA algorithm is ClustalW2.

The target protein, homologous protein, complementary protein or peptide ligand, disclosed hereinabove, binds a nucleic acid. The present invention further includes a computer program embodied on a computer readable medium, a computer system, comprising programming, a computer storage media, comprising programming or any combinations thereof to perform the method described hereinabove. The present invention also discloses composition comprising one or more novel peptides capable of binding to the target protein, DNA, or both identified by the method provided herein. Finally, the invention presented herein provides an optimized binding agent or binding agent composition from multiple peptides discovered by the method of the present invention or as otherwise identified, wherein the agent targets several different protein domains and comprises several short peptides that are linked.

### BRIEF DESCRIPTION OF THE DRAWINGS

For a more complete understanding of the features and advantages of the present invention, reference is now made to the detailed description of the invention along with the accompanying figures and in which:

FIG. 1 shows the step-wise process to HALO/Gen. Computerized datamining is carried out from primary sequence of the target protein in order to determine sequence homologous proteins. This is followed by computerized textmining of public and literature databases (available on LiBase) for candidate peptide ligands (SEQ ID NOS 306, 309, 314, 313, 310, 342 and 305, respectively, in order of appearance) to the target protein. Automated ligand walking and sequence expansion using custom designed amino-acid substitution tiers is enabled by HALO/Gen to help generate customized peptide microarrays for a target protein. These are used to screen iteratively against the target protein to identify and mature high-affinity peptide binders to the protein;

FIG. 2 represents a stepwise workflow of the HALO/Gen process of the present invention. The primary sequence of a query protein is used to data mine for homologous sequence proteins (HSPs) using various existing algorithms and databases. These are then used to query open source biomedical literature and other databases for candidate peptide ligands that will have affinity for the query protein. Iterative sequence expansion and screening are carried out for affinity maturation to lead to custom designed, selective, high-affinity peptide ligands which can be incorporated into various niche applications including therapeutics development and biomarker ligands;

FIG. 3 shows the results from an in vitro TR-FRET ESRRG-ligand displacement assay for 3 peptide (SEQ ID NOS 233, 210 and 275, respectively, in order of appearance) inverse agonists compared to the positive control, 4-hydroxytamoxifen (4-OHT). The average of four replicates is shown for each concentration. Error bars show one SD for the replicates. Large open squares on the 4-parameter fitted dose response curve show the EC₅₀ for each peptide. (The three peptides are plotted against the secondary y-axis on the right). The EC50 of peptides ranged from 2-fold to 10-fold that of 4-OHT);

FIGS. 4A-4C show the consensus charts of input sequences: (FIG. 4A) on the peptide microarray and top high-affinity ligands to ANXA1 (FIG. 4B) and ANXA5 (FIG. 4C). Central 2 amino acids of 8-mer parent peptides were permuted using a unique matrix of functionally similar amino acid substitutions (A-G; do not represent actual amino acids. See Table 14). Primary screening results show the selective evolution of ligands to each protein derived from the same condensed ligand space. The motif showing highest affinity for ANXA1 was YLDxxAGN (SEQ ID NO: 330) compared to SERzzARY (SEQ ID NO: 331) for ANXA5 on the same array;

FIG. 5 shows the results of simultaneous screening of orthogonally fluorescently labeled ANXA1 (Cy5) and ANXA5 (Cy3) against a common peptide array (13K peptides) derived from shared candidate ligands between ANXA1 and ANXA5. FIG. 5 represents another approach to development of sensitive and specific peptide ligands to proteins. The consensus chart of ligand space from all input peptide ligands is shown on top; the consensus peptide space of top 100 ligands to ANXA1 and to ANXA5 is listed below. Parallel screening against a shared array led to selection for several ligands (>100) that bound uniquely to only ANXA1 or ANXA5. Further iterative arraying and screening is projected to lead to maturation of ligands with unique specificity and sensitivity for either protein;

FIG. 6 is a schematic representation of the sequence details for the SMRT Peptide TNMGLEAIRKALMGKYDQWEE (SEQ ID NO: 6) identified through text mining to bind to ESRRG or its sequence homologous protein(s);

FIG. 7 is a schematic representation of the sequence details for the nuclear receptor coactivator 2 peptide KHKILHRLLQDSS (SEQ ID NO: 80) identified through text mining to bind to ESRRG or its sequence homologous protein(s);

FIG. 8 is a schematic representation of the sequence details for the TIF-2 Box 3 peptide KENALLRYLLDKDD (SEQ ID NO: 74) identified through text mining to bind to ESRRG or its sequence homologous protein(s); and

FIG. 9 represents an example of approach to text mining of captions within biomedical literature to assist a user in identifying candidate ligands to the query or its sequence homologous protein(s). FIG. 9 discloses the left column “a” peptides as SEQ ID NOS 343-360, respectively, in order of appearance, and the right column “a” peptides as SEQ ID NOS 361-389, respectively, in order of appearance.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Protein targets that are relevant to the present invention include cell membrane receptors, nuclear membrane receptors, intracellular, nuclear, and mitochondrial proteins including nuclear transcription factors, secreted proteins, circulating peptide and non-peptide receptors, membrane and circulating transporters, enzymes, chaperonins and chaperonin-like proteins; antibodies, surface and intracellular proteins of infectious agents, and more generally, any protein involved in peptide-protein, protein-protein, and/or protein-DNA interactions. The protein targets may be naturally present in humans or may be a natural component of other species. The protein target may not necessarily be naturally occurring. Some protein targets may have been generated by artificial means. The peptides resulting from this invention are selected to selectively bind to and/or detect, or otherwise modulate, activate and/or inhibit the function of the target protein.

The invention makes use of the fact that there is an enormous amount of scientific and technical information, published journal articles, technical papers, and computer databases regarding the interaction of peptides and peptide like molecules with proteins. This information is generally of limited use in predicting peptide or protein interactions. The invention teaches a newly discovered process for extracting and correlating scientific information about proteins and peptides to discover new peptides with desired binding characteristics. It is noteworthy that there exist catalogs or databases that collect and classify information on ligand molecules that are known to interact with proteins. Examples of such collection of information are the PepBank database (pepbank.mgh.harvard.edu/search/basic) and the antimicrobial peptide database (aps.unmc.edu/AP/main.php). The present invention is distinct from these types of catalogs. These catalogs record information from public sources, publications and report, but do not teach or describe methods or processes to identify or predict and discover new peptide ligands with a specific protein target in mind.

The present method is particularly applicable when the structure of the target is unknown, as the only requirement to perform the invention is knowledge of the linear amino acid sequence of the target. An artisan would appreciate that the method is also applicable to targets whose tridimensional structure is known, by using the primary sequence or part of the primary sequence of the target protein as input. In some instances, the process may be applied knowing only partial sequence information about the target protein rather that the amino acids sequence in its entirety. The invention includes the discovery of peptides or peptide-like molecules which have a high probability of binding to and/or otherwise modulating the function of polypeptides or proteins, and to methods for designing and validating and improving the functionality (binding characteristics) of such peptides or peptide-like molecules. The discovery of high-affinity peptide ligands disclosed here uses a novel procedure that narrowly selects ligand candidates to a target or query protein from an initial input of only the amino acid sequence of the target or query protein.

The process of the invention begins by obtaining the amino acids sequence of the target protein. Often the target may have been previously studied to the extent that the primary amino acid sequence will be known. Many resources are available from which the amino acid sequence information on the protein can be obtained. There may be occasions when the target protein is new and not previously been sequenced. In this instance, there is a requirement to sequence the unknown protein to determine its sequence. It may not be necessary to determine the sequence in its entirety, knowledge of only portions of the sequence may be adequate. There are standard laboratory methods for determination of the amino acid sequence of a protein. Once the amino acids sequence is obtained, the invention requires a specific form of search to be carried out. Specifically, we search for proteins that have amino acids sequence homology with the target protein. For the purposes of this invention, we define an homologous protein as a protein that possess an amino acid sequence that matches or partially matches the amino acid sequence found in the target or query protein (for example 20%, 30%, 40%, 50%, 60%, 70%, 80%, 90% or 100% homology).

One method for finding protein with sequences that match or are similar to the target is to use a BLAST (Basic Local Alignment Search Tool) or similar protocol. A BLAST search (blast.ncbi.nlm.nih.gov/Blast.cgi) is carried out using the protein sequence of the target protein. The non-redundant protein database can be selected for the BLAST query because it comprises proteins from all other databases, including SwissProt, PDB, PIR, and PRF. Open source protein databases to be queried may include the Protein Databank (PDB) of protein structures, RefSeq, and UniProt (European Bioinformatics Institute consortium).

The threshold E (expectation) value for the query can be varied but generally is set to 0.1 or lower (for e.g., 0.01, 0.001, 0.0001). E-value specifies stringency of the threshold or cut off. The higher the threshold, the greater possibility that homology may not be real but ‘accidental’. Gap values can be changed to increase or decrease stringency, and the matrix used may also be changed (e.g., PAM or BLOSUM). These are all part of a BLAST query and available to one trained in the arts.

In one embodiment of the present invention, the BLAST search for each protein provides a dataset of homologous proteins ranked by their total gap scores and E-value. Position-specific iterative BLAST (PSI-BLAST) may be used to provide a large hit set containing more remotely related proteins. Another way of determining homologous proteins to the target protein is through using InterPro (http://www.ebi.ac.uk/interpro/). InterPro combines a number of member databases (including Pfam, ProSite, PRINTS, and Propom) that use different methodologies and a varying degree of biological information on well-characterized proteins to derive protein signatures such as families, domains, repeats, and sites. InterPro permits searching the database by protein sequence (e.g. FASTA) using InterProScan or by protein name (keyword search). The results from an InterPro search lists proteins sharing domains, repeats, and functional sites broken down by ‘parent/child’ and ‘contains/found-in’ relationships to the query. By this approach additional datasets of homologous proteins may be derived. It will be understood by the skilled artisan that other databases for domain searching may be used (for e.g. CDD and CDART databases).

Persons of ordinary skill will recognize there are multiple alternative search methods to achieve the same goal of identifying proteins that containing amino acid sequences that match exactly or in part, sequences possessed by the target protein. Additional methodologies may become available in the future as computerized record keeping, data storage and retrieval, and web based search engines continue to evolve.

Many protein-protein interactions are mediated by small protein modules binding to short linear peptides in their protein complement. The interaction map of both eukaryotic and prokaryotic proteins is being documented using various studies, and various public source databases are available from which to obtain this information. These include but are not limited to DIP (dip.doe-mbi.ucla.edu) and IntAct (www.ebi.ac.uk/intact/). These databases are used to collate information about other proteins that are known to interact with the query or homologous proteins. Textmining can be used to infer peptide sequences present at these protein-protein interaction sites that could serve as potential leads for optimization.

After identifying homologous proteins and complement proteins (i.e., proteins that interact with target protein and homologous proteins to the target), the invention involves curating information contained in publications, literature reports, documents and computerized records for the purposes of identifying peptide ligands that bind to the homologous proteins. The invention teaches that ligands that are able to bind to the homologous protein might also be expected to have a binding affinity for the target protein. This is because the target protein and the homologous protein have amino acid sequences in common. Then, if the ligand binds to the homologous protein at point where the amino acids sequence is similar to the sequence found in the target protein, that same ligand may be able to bind to the target protein (e.g. the ligand will have some degree of affinity for the target protein, which can, optionally, be optimized). In this way, we may reasonably expect to indirectly identify a narrow range of candidate molecules with the desired binding properties to the target.

In one embodiment, the required text searching can be performed manually by reading the relevant documents and publication describing studies of the homologous protein, specifically for the purposes of seeing if peptide ligands to the homologous proteins have been described. This information is contained in diverse databases including abstracts, scientific journals, and other public sources. A problem to address is that multiple documents have to be read and screened to determine if they contain the desirable subject matter. Specifically, the reader would reasonably be familiar with protein chemistry and the associated scientific nomenclature and would be able to correctly identify passages of text, items in tables or figures where peptide sequences have been included. The search is further complicated because of the need to search by protein ‘name’ as well as using synonyms and commonly accepted acronyms. Thus, manually identifying potential ligand binding partners to the datasets of homologous proteins is a significant challenge.

A preferred embodiment would be to use computer assisted text searching, employing links to publicly accessible databases, such as MedLine and PubMed Central (PMC, www.pubmedcentral.nih.gov), although the invention is not confined to these data bases. Private or commercial data bases can be used. These sources are structured in a way that is protracted but simple for humans to read and comprehend but difficult for computers to interpret. In such a computerized approach, scientific literature is queried, using customized search terms, for references to peptide ligands, interacting domains, motifs, sequences, epitopes, etc., to proteins identified from the homologous protein search aspect of the invention. Queries are also made for published sequences within the DIP (Database of interacting proteins) and IntAct (Interacting proteins database) proteins that ‘bind’ to their corresponding partners.

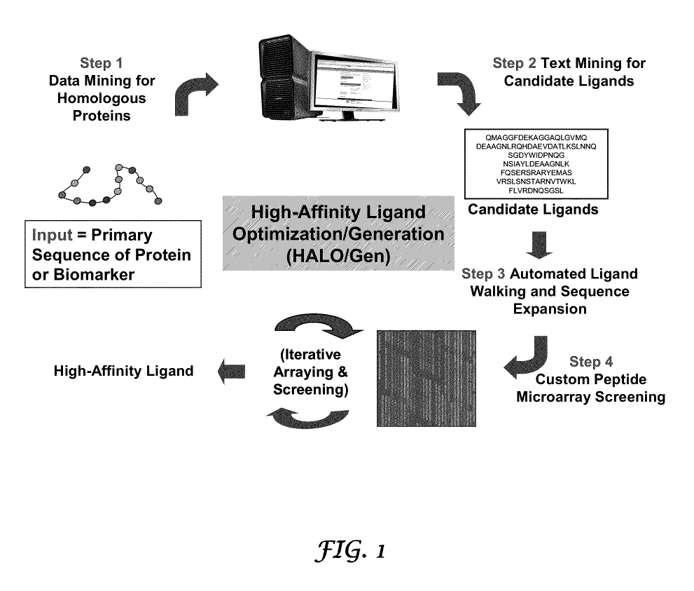

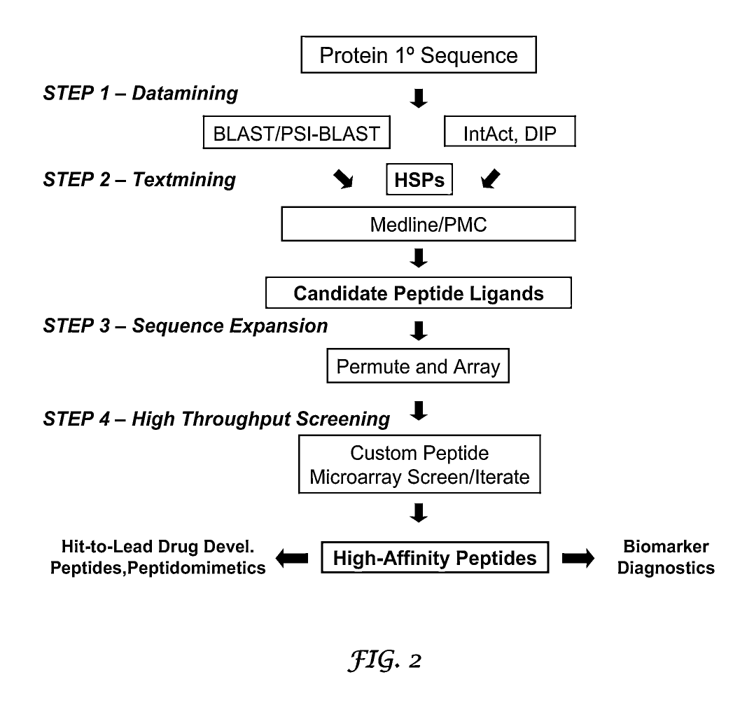

Preferably, the new computer-driven text mining software program HALO can be used to speed this aspect of the invention (FIG. 1). This engine was designed to identify peptide, peptide-like, and peptide-related terms (referred to as peptides) using a customized query process combining protein names, synonyms, keywords, and commonly co-occurring terms in the vicinity of the peptides. A stepwise workflow of the HALO/Gen process of the present invention is shown in FIG. 2.

Typically, the process described above identifies several (>100) and preferably 10-20 peptide sequences as candidates, to be further assayed for binding or for their ability to modulate or affect target protein activity. This is a custom designed sample ligand space for the protein, in marked contrast to the usual drug discovery methods which can require synthesis and screening of vast unfocussed libraries of randomly selected compounds. At this point in the process, it is not known if the candidate peptides have the desired property, i.e., affinity, selectivity, specificity, etc., for the target protein. Therefore, once candidate ligands have been identified, their affinity and specificity for the target protein is optimized experimentally.

The invention also embodies methods to improve and refine the protein binding properties (selectivity, specificity, affinity, etc.) of the candidate peptides. A process of amino acid “sequence modulation” may be performed, if required. Here the inventors provide an example. From the set of candidate ligands to a query protein, all sequences longer than 8 (or 10 or 12)-amino acids are subjected to ligand walking (a process similar to epitope walking) to obtain an n-mer (n=8-12) super-library of candidate ligands. The reasons for this are described herein:

It has been demonstrated that linear sequences of ˜5-6 amino acids long are typically sufficient to provide significant interaction with a protein (McEntyre and Gibson 2004) since the binding energy for a ligand-protein is not distributed evenly across their interface but is localized to small regions of the protein of less than five amino acids that represent energetic focal points of the interaction (Arkin and Wells 2004). The yield of the step-wise, divergent synthesis of peptide ligands on the array is not quantitative (ca. 95-97%), and sequences longer than 10 amino acids tend to lose sequence fidelity.

Then, a matrix of peptides comprising ‘knowledge based’ permutations of the core amino acids (central 2 or 4 or ‘n’) of each n-mer (8-mer taken as example) peptide in the super-library are created initially for arraying, using a substitution matrix such as BLOSUM (Henikoff and Henikoff 1992), as these residues are generally predicted to contribute maximally to protein-protein or protein-peptide binding interactions. The BLOSUM matrix is derived from complex mathematical analysis of the evolutionary design of proteins that have elucidated the most commonly observed point-mutations between protein structure families. For example, acceptable permutations might include Gly [symbol] [symbol] Ala [symbol] Val [symbol] [symbol] Leu [symbol] Ile, Phe [symbol] [symbol] [symbol] Tyr [symbol] Trp, Lys [symbol] [symbol] [symbol] [symbol] Arg, Glu [symbol] [symbol] [symbol] [symbol] Asp, Thr [symbol] [symbol] [symbol] [symbol] Ser, Gly [symbol] [symbol] [symbol] [symbol] Pro, etc. It is recognized that there are multiple ways to perform these substitutions and variation to the candidate ligands. An artisan would also appreciate that longer or shorter than 8 amino acids may be used, and that the acceptable combinations and permutations may involve more or less than 2 amino acids in contiguous or non-contiguous positions. Uncommon substitutions, referred to as high-penalty substitutions as defined by BLOSUM, may also be used to generate sequences not commonly found in nature.

High-throughput screening is critically important at this stage, because although a relatively narrow ligand sample space has been provided, it is reasonable to expect automated text-mining to identify a certain number of false positives for a variety of reasons, such as finding nonsense or non-binding peptide sequences. Moreover, since sequence space can expand rapidly (following 20ⁿ, where n is the length of the ligand), being able to increase the number of ligand molecules screened at a time is highly desirable. Screening involves synthesis of the candidate ligands, and its rationally expanded ‘sequence space’ analogs, and directly testing their binding affinity towards the target. The value of the technique is thus enhanced further through systematic (rather than random all-for-all) variations in the binding ligand's chemistry (i.e. peptide sequence) to modulate the binding efficiency, as described herein.

Optimization of ligands for protein binding may be performed in numerous ways. In order to facilitate screening of the target protein against several thousand fine-tuned variations of our ligands simultaneously, for example, high-throughput screening can be achieved using a high-density custom microarray in which arrays of candidate ligands (and their permutations) customized for the target protein are built from the ground up using a state of the art automated peptide arraying technique. It will be understood by a skilled artisan that alternative methods may also be used to synthesize and screen peptide ligands in parallel, including other peptide microarray methodologies (for e.g., PepSpot, inkjet printing), combinatorial library of peptides, and phage display. Affinity constants may also be determined using any number of methods, including on custom peptide microarrays. Iterative screening is often carried out to further refine ligand binding properties (affinity, selectivity, specificity) desirable for the required application.

For iterative screening, sequence modulation of the ligands may be carried out as follows. The core (2 or 4) amino acids of the ligand from the primary screening are kept unchanged while pairs of outlying amino acid residues (e.g., positions 1 & 8 and 2 & 7) in the sequence are permuted using BLOSUM based substitutions. This approach allows a cipher lock-like systematic tuning of the ligand interaction of the high-affinity 8-mer from the primary screen with its target ‘epitope’ in order to refine and optimize its affinity. Alanine scanning to determine immutable amino acids can also assist in controlled expansion of the ligand space.

The methodology, when successfully implemented, can significantly advance existing methods for generating high affinity ligands for targeting protein-protein interactions. Automation of this process into a bioinformatics program has been completed, which can rapidly customize or refine the candidate ligand space to be screened will vastly expedite the process of hit-to-lead discovery, and it will provide a handy tool in the arsenal of drug discovery research. Once an active ligand has been identified, further studies may be required to determine e.g. mode of interaction, potential side effects, and possibility of improving the drug-characteristics of the ligand. A notable advantage to our approach in addition to the rapid turn around from protein sequence to peptide ligand is that the performance of our process can be continually enhanced as more proteins and their ligands are added to the existing databases (including through our screening and iteration process).

### Example I

### Estrogen-Receptor Related Receptor Gamma

ESRRG (Estrogen-receptor related receptor gamma, a.k.a., ERRγ, ERR gamma, ERR3) is involved in transcriptional activation of gene expression. ESRRG has been proposed to be a prognostic breast cancer biomarker indicative of clinical outcome and sensitivity to hormonal therapy, and it is a putative therapeutic target in prostate cancer.

The ERR proteins (comprising ERR α, β, and γ) are ubiquitous, constitutively active, orphan members of the nuclear receptor superfamily that display a wide range of cell-specific gene transcriptional activities which contribute to cell maintenance, apoptosis, and metabolic pathways (Ariazi and Jordan 2006). The ERRs are closely related to the ERs, specifically within their DNA-binding (DBD) and ligand-binding (LBD) domains. Due to their shared DBD homology, ERRs are known to transactivate ERα genes through estrogen response elements (EREs). There is strong evidence suggesting that the control of overlapping gene regulatory pathways plays a role in cancer therapy response of ER+ breast cancer cells. ERRs also transcribe a different set of genes, through a unique ERR response element (ERRE), that control aspects of plasma membrane generation and the synthesis of steroid hormones, which could augment proliferation rate of tumor cells (Anderson and Clarke 2004; Clarke, Anderson et al. 2004). ESRRG was shown to directly mediate Tamoxifen resistance in a representative invasive lobular carcinoma cell model by activating transcription through cis-acting AP-1 sites (similar to ERα), but only when bound by Tamoxifen (Riggins, Lan et al. 2008). AP-1 activity is significantly upregulated in Tamoxifen-resistant cells (Dumont, Bitonti et al. 1996; Johnston, Lu et al. 1999; Schiff, Reddy et al. 2000; Zhou, Yau et al. 2007).

In a recent study (Galindo, McCormick et al. 2010), a custom oligo-array measuring global microsatellite content among individual genomic DNA samples has determined an unique and reproducible pattern of 26 motif-specific microsatellite families that specifically characterize breast cancer, and 4 motif families that were cancer-type specific. The same motifs were found in tumor DNA of the same patients but not in germline DNA samples from healthy volunteers. The results indicate that some cancer patients might possess variable numbers of microsatellites that are predictive of future cancer development. A key determination was that a variable-length microsatellite in the 5′-UTR promoter region of the ESRRG gene may correlate with heritable cancer risk. Of the 22 candidate transcription factors that potentially could bind to this region of ESRRG containing the repeat (plus 100 bp flanking sequences), one of which (paired box 2, PAX2) is capable of binding the repeat unit itself. This finding suggests another potential mechanism of action, as PAX2 was recently implicated in ER-mediated regulation of ErbB2 (Her2, EGFR2) and resistance to Tamoxifen (Hurtado, Holmes et al. 2008).

Thus, there is mounting evidence for an important role for ESRRG in breast cancer etiology and therapy response, and as a putative biomarker for breast cancer prognosis.

Step 1: Datamining

Protein Sequence

Objective—To obtain the protein or biomarker primary sequence.

Several sources for primary sequence information exist, including protein databases, (e.g., PDB, Human Mitcohondrial Protein Database, AMPer, UniProt, etc.), Mass Spectrometry based methods (e.g., SEQUIT), methodologies based on DNA translation), among others.

FASTA sequence for ESRRG was obtained from Protein Databank

ESRRG → 1VJB (PDB ID) >1VJB: B|PDBID|CHAIN|SEQUENCE
(SEQ ID NO: 1)
MGSSHHHHHHSSGLVPRGSHMPAKKPYNKIVSHLLVAEPEKIYAMPDPTVPDSDIKALTTLC
DLADRELVVIIGWAKHIPGFSTLSLADQMSLLQSAWMEILILGVVYRSLSFEDELVYADDYIM
DEDQSKLAGLLDLNNAILQLVKKYKSMKLEKEEFVTLKAIALANSDSMHIEDVEAVQKLQD
VLHEALQDYEAGQHMEDPRRAGKMLMTLPLLRQTSTKAVQHFYNIKLEGKVPMHKLFLEM
LEAKV
>1VJB: A|PDBID|CHAIN|SEQUENCE
(SEQ ID NO: 1)
MGSSHHHHHHSSGLVPRGSHMPAKKPYNKIVSHLLVAEPEKIYAMPDPTVPDSDIKALTTLC
DLADRELVVIIGWAKHIPGFSTLSLADQMSLLQSAWMEILILGVVYRSLSFEDELVYADDYIM
DEDQSKLAGLLDLNNAILQLVKKYKSMKLEKEEFVTLKAIALANSDSMHIEDVEAVQKLQD
VLHEALQDYEAGQHMEDPRRAGKMLMTLPLLRQTSTKAVQHFYNIKLEGKVPMHKLFLEM
LEAKV

Synonyms—a.k.a. ERR3, ERRG2, KIAA0832, NR3B3 (UniProtKB)

Alternate sources of obtaining protein synonyms include, but are not limited to, biomedical literature (Entrez PubMed, BIOMINT (gene and protein synonym database, 42 synonyms for ESRRG), UniProt, PDB.

Gene Information: Gene information for ESRRG was obtained from Entrez. This information also is available from sundry other databases within Entrez (GenBank, UniGene, etc.). Although this information was not directly relevant to the ligand discovery process for ESRRG, it offers useful insights/information about target proteins.
Entrez GeneID: 2104
GeneBank Accession#: NM_{—}206594
Protein Accession#: NP_{—}996317.1
Gene Name: ESRRG
Gene Alias: DKFZp781L1617, ERR3, NR3B3
Gene Description: estrogen-related receptor gamma
Omim ID: 602969

Step 1.1: Dataset of Homologous Proteins 1 (DHP1)

Objective: To determine proteins sharing sequence homology to entire protein and domains within it (if known) using BLAST/PSI-BLAST/FASTA etc. (similar information may be obtained through InterPro, CDD, CDART, or other protein motif, domain or family database).

Primary sequence of ESRRG was BLASTed using iterative PSI-BLAST with the following parameters—E (expectation threshold)=0.001 (0.005 for PSI BLAST); Max. target sequences=500; with all other parameters (word size, matrix, etc.) at ‘default’, against the NR (non-redundant protein) database.

Information of known domains or regions within ESRRG—e.g., Ligand-binding (LBD, 220-442) and DNA-binding (DBD, 121-228) domains—was obtained from the CDD (also available through UniProt, BLAST, BLink, etc.). These were also subjected to independent PSI-BLAST queries.

BLAST (entire protein, 458 aa), E = 0.001, NR database, 500 max. hits
ER (Estrogen receptor)
Nuclear receptor subfamily 2 & 3
Interleukin 12a p35
Human Retinoid-X-Receptor/Retinoic acid receptor gamma (RXR gamma)
(similar to) Phosphomevalonate kinase
(similar to) hERRB2 (human estrogen related receptor beta 2)
Chicken ovalbumin upstream promoter (COUP)-transcription factor (1, 2)
Seven Up, isoform C
Progesterone receptor
Glucocorticoid receptor/Mineralocorticoid receptor
BLAST (fragment 121-240-includes DBD)-(Additional to above)
Retinoic Acid Receptor (RAR) alpha
Ultraspiracle (USP) protein ligand binding domain
MHC Class I Promoter Binding Protein
Steroid hormone receptor ERR2
Liver Hormone Receptor (LRH), Liver-X Receptor
FTZ-F1 (Fushi-Tarazu Factor 1)
BLAST (fragment 220-440-includes LBD)-(Additional to above)
ESRRB/A/D (estrogen related receptor beta, alpha, and delta)
Estrogen receptor isoforms (alpha, beta1, beta2)

Step 1.2: Dataset of Homologous Proteins 1 (DHP2)

Another way of identifying homologous proteins to the target protein is through InterPro (www.ebi.ac.uk/interpro/). InterPro combines a number of member databases (including Pfam, ProSite, PRINTS, and Propom) that use different methodologies and a varying degree of biological information on well-characterized proteins to derive protein signatures such as families, domains, repeats, and sites. InterPro permits searching the database by protein sequence (e.g., FASTA) using InterProScan or by protein name (keyword search). The results from an InterPro search lists proteins sharing domains, repeats, and functional sites broken down by ‘parent/child’ and ‘contains/found-in’ relationships to the query protein.

An InterPro query for ESRRG was carried out starting from its FASTA sequence (i.e., using InterProScan).

| InterPro ID | Protein Name | |
|---|---|---|
| IPR000003 | Retinoid X receptor | |
| IPR001728 | Thyroid hormone receptor | |
| IPR003068 | Transcription factor COUP | |
| IPR003069 | Ecdysteroid receptor | Children |
| IPR003074 | Peroxisome proliferator-activated receptor | |
| IPR003078 | Retinoic acid receptor | |
| IPR003079 | Nuclear receptor ROR | |
| IPR016355 | Fushi-tarazu (FTZ) factor 1 | |
| IPR000536 | Nuclear hormone receptor, ligand binding, core | |
| IPR008946 | Nuclear hormone receptor, ligand binding | Contains |
| IPR013629 | Zinc finger associated region | |

Step 1.3: IntAct/DIP (Database of Complementary Proteins)

Objective: To determine proteins that are known to bind to query (target) protein and its homologs. This information assists in focused textmining for protein-protein interacting sequences.

Many protein-protein interactions are mediated by small protein modules binding to short linear peptides in their protein complement. IntAct and DIP (Database of Interacting Proteins) are two well known such databases; other similar databases may also exist. These afford a way to determine interacting partners to a target query protein, which can assist in determining interacting linear peptide sequences within these partners, through customized text mining, which are suitable as starting points for ligand discovery.

Proteins that interact with ESRRG and its homologs (e.g., ER, RXR, etc. from DHP1 and DHP2 above) were queried using IntAct. Some of this information is detailed below. In examples below, ER interacts with FOXO3 (Forkhead box Protein O3) and FOXM1; likewise, RXR interacts with a set of other proteins, including RAR alpha and PPAR alpha; and IL12a p35 unit interacts with IL12b (receptor subunit b). Other proteins were datamined in similar fashion (examples not shown).

1.3.1 ER (Nuclear Hormone Receptor)

| | | Number of | | Gene | |
|---|---|---|---|---|---|
| IntAct name | IntAct Ac | interactions | Identifier | name | Description |
| ERalpha_humandna | EBI-1657542 | 2 | — | — | Human estrogen |
| | | | | | receptor alpha |
| | | | interacts with | | |
| foxo3_human | EBI-16441641 | 1 | O43524 | FOX03 | Forkhead box |
| | | | | | protein O3 |
| foxm1_human | EBI-866480 | 1 | Q08050 | FOXM1 | Forkhead box |
| | | | | | protein M1 |

1.3.2 RXR (Retinoid X Receptor)

| | | No. of | | | |
|---|---|---|---|---|---|
| | IntAct | inter- | | | |
| IntAct name^{?} | Ac^{?} | actions | Identifier | Gene name | Description |
| RXRg_human | EBI-712405 | 5 | P48443 | RXRG | Retinoic acid receptor |
| | | | | | RXR-gamma |
| | | | interacts with | | |
| scg1_human | EBI-712619 | 1 | P05060 | CHGB | Secretogranin-1 |
| rara_human | EBI-413374 | 1 | P10276 | RARA | Retinoic acid receptor |
| | | | | | alpha |
| pno1_human | EBI-712787 | 1 | Q9NRX1 | PNO1 | RNA-binding protein |
| | | | | | PNO1 |
| ppara_human | EBI-78615 | 1 | Q07869 | PPARA | Peroxisome |
| | | | | | proliferator-activated |
| | | | | | receptor alpha |
| lrsm1_human | EBI-720984 | 1 | Q6UWE0 | LRSAM1, | E3 ubiquitin-protein |
| | | | | UNQ6496/ | ligase LRSAM1 |
| | | | | PRO21356 | |

1.3.3 IL-12a p35 Subunit (IL12a)

| | | Number | | | |
|---|---|---|---|---|---|
| | | of inter- | | Gene | |
| IntAct name | IntAct Ac | actions | Identifier | name | Description |
| il12a_human | EBI- | 1 | P29459 | IL12A | Interleukin- |
| | 1029636 | | | | 12 subunit |
| | | | | | alpha |
| | | | interacts with | | |
| il12b_human | EBI- | 1 | P29460 | IL12B | Interleukin- |
| | 1029614 | | | | 12 subunit |
| | | | | | beta |

Step 2: Textmining (PubMed Central/PubMed/MedLine)

Objective—To identify known peptide ligands, sequences, motifs, paratopes, etc., within the medical literature database through custom combination keyword and co-occurring term queries.

ESRRG and its homologous proteins were textmined by interrogation of the biomedical literature database using custom keyword query strings (see Table 1 below) followed by manual curation of the text to collate peptide ligands, interacting linear sequences, phage display ligands, etc. Typically, query strings comprised a combination of terms from Table 1 below. For example, “Term 1 AND Term 2 AND Term 3”; “Term 1 AND Term 2 AND Term 4”; “Term 1 AND Term 3 AND Term 4”; “Term 1 AND Term 4”; and so on.

TABLE 1
List of some query keywords and combinations used for textmining a
biomedical literature database for candidate ligands to a target protein.

| Term 1 | Term 2 | Term 3 | Term 4 |
|---|---|---|---|
| Target Protein or | Binding, | Peptide, ligand, | Interacting |
| Homolog, synonyms, | Interacting, | sequence, motif, | Proteins, |
| common | Interaction | region, domain, | Synonyms, |
| abbreviations | | epitope, paratope, | common |
| | | etc. | abbreviatons |

A sampling of the queries attempted is provided in the following sections, along with context within text (in italics, within “double quotes”), reference (author, publication title in ‘single quotes’, journal reference, etc.), PMID# or PMCID#, and any other relevant details. An exhaustive search can now be carried out using our automated datamining portal Libase on the HALO/Gen website.

Query 1: Estrogen Receptor+Peptide Ligand

ESRRG #01: RIP40 peptide (“LERNNIKQAANNSLLLHLLKSQTIP”) (SEQ ID NO: 2)—(also a PDB entry).

Wang, L.; et al. ‘X-ray crystal structures of the estrogen-related receptor-gamma ligand binding domain in three functional states reveal the molecular basis of small molecule regulation’. J Biol. Chem. 2006 Dec. 8; 281(49):37773-81. Epub 2006 Sep. 21. PMID: 16990259 (ESRRG #01).

ESRRG #02: “RHKILHRLLQEGSPS” (SEQ ID NO: 3)—Steroid receptor coactivator 1 (PDB entry 1 KV6; ‘X-ray structure of the orphan nuclear receptor ERR3 ligand-binding domain in the constitutively active conformation’. PMID: 11864604 (ESRRG #02).

ESRRG #03: “KHKILHRLLQDSS” (SEQ ID NO: 4)—Steroid receptor coactivator 3 (PDB entry: 1×7R; ‘Understanding the selectivity of genistein for human estrogen receptor-beta using X-ray crystallography and computational methods’. PMID: 15576033, (ESRRG #03).

ESRRG #04: “SGSHKLVQLLTTT” (SEQ ID NO: 5)—WAY 697 (PDB entry: 1×76; ‘Structure-Based Design of Estrogen Receptor-Beta Selective Ligands’. J. Am. Chem. Soc. 126: 15106-15119. PMID: 15548008 (ESRRG #04)

ESRRG #05: SMRT Peptide “TNMGLEAIRKALMGKYDQWEE” (SEQ ID NO: 6) (see FIG. 6)

SRC-1 NR box 4 peptide (ERβ)—“QAQQKSLLQQLLTE” ((SEQ ID NO: 7);

SRC-1 NR box 2 peptide (ERα)—“LTERHKILHRLLQEGSPSD” (SEQ ID NO: 8);

cyclo-K^{D}CILCRLLN (PERM-1) (SEQ ID NO: 9)—See Table 2.

Leduc, A. M. et al. ‘Helix-stabilized cyclic peptides as selective inhibitors of steroid receptor coactivator interactions’. PNAS 2003, 100(20), 11273-8. PMID: 13679575 (ESRRG #05).

TABLE 2
Table from a literature reference. Inhibition of estrogen receptor/coactivator
recognition as assessed by a time-resolved fluorescence-based coactivator
interaction assay (from ESRRG #05) (structures 1-10 disclosed as
SEQ ID NOS 332-334, 4, 335-338, 8 and 121, respectively).
Inhibition of estrogen receptor/coactivator recognition as assessed by a
time-resolved fluorescence based coactivator interaction assay (24)

| | | K, values, μM | | |
|---|---|---|---|---|
| | Structure | ERα | ERβ | ERβ/ERα |
| 1 | H-Leu-Glu-Gln-Leu-Leu-OH | not active | 201 | ND |
| 2 | Mannosylacetyl-Leu-Glu-Gln-Leu-Leu-OH | >200 | >200 | ND |
| 3 | H-Lys-Lys-Ile-Leu-His-Arg-Leu-Leu-Gln-NH₂ | 0.17 | 2.8 | 16.5 |
| 4 | H-Lys-His-Lys-Ile-Leu-His-Arg-Leu-Leu-Gln-Asp-Ser-Ser-OH | 0.38 | 6.9 | 18.1 |
| 5 | 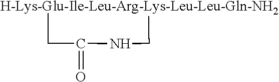 | 0.22 | 4.8 | 21.8 |
| 6 | 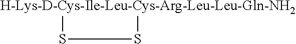 | 0.025 | 0.39 | 15.6 |
| 7 | 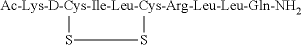 | 0.12 | 7.7 | 64.2 |
| 8 | 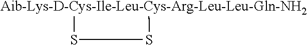 | 0.13 | 1.4 | 10.8 |
| 9 | SRC-1 NR2 (LTERHKILHRLLQEGSPSD) | 0.70 | 5.2 | 7.4 |
| 10 | SRC-1 NR4 (QAQQKSLLQQLLTE | 0.45 | 1.1 | 2.4 |

ND, not determined.

ESRRG #06: GGSCPSSHSSLTERH—(SEQ ID NO: 10) (“residues 683-697 of Swiss-Prot entry Q15788”).

Jin, K. S., et al. ‘Small-angle X-ray scattering studies on structures of an ERRalpha ligand binding domain and its complexes with ligands and coactivators’. J. Phys. Chem. B 2008, 112(32), 9603-12. PMID: 18646811 (ESRRG #06)

Abstract—“We also synthesized steroid receptor coactivator-1 (SRC-1), a 15-mer peptide corresponding to the leucine-rich repeat 4 of human SRC-1”.

Results and Discussion, paragraph 1—“In addition, SRC-1, a 15-mer peptide corresponding to the leucine-rich repeat 4 of human SRC-1 (residues 683-697 of Swiss-Prot Entry Q15788), was synthesized”.

ESRRG #07: QQQKPQRRPCSELLKYLTTNDD—(SEQ ID NO: 11)—PPAR Gamma Coactivator-1 (PGC-1) Box 3 peptide.

KENALLKYLLDK—(SEQ ID NO: 12)—TIF-2 Box 3 peptide

Greschik, H., et al. J. Biol. Chem. 2008, 283(29), 20220-30. PMID: 18441008 (ESRRG #07)

“We co-crystallized the ERRa-LBD with a “wild-type” PGC-1α Box3 peptide (¹⁹⁸QQQKPQRRPCSELLKYLTTNDD²¹⁹)—(SEQ ID NO: 13) whereas in the case of the reported structure a shorter, mutant peptide (²⁰⁵RPASELLKYLTT²¹⁶; C207A) (SEQ ID NO: 14) was used”.

| | | |
|---|---|---|
| A) PGC-1α | L1 DEENEANLLAVLTETLDSL | SEQ ID NO: 15 |
| | L2 EAEEPSLLKKLLLAPANTQ | SEQ ID NO: 16 |
| | L3 QRRPCSELLKYLTTNDDPP | SEQ ID NO: 17 |
| B) SRC2 | L1 DSKGQTKLLQLLTTKSDQM | SEQ ID NO: 18 |
| | L2 LKEKHKILHRLLQDSSSPV | SEQ ID NO: 19 |
| | L3 PKKKENALLRYLLDKDDTK | SEQ ID NO: 20 |

“A favorable contribution of Arg-205, Pro-206, and Cys-207 to the ERRα/PGC-1α interaction is also interesting with respect to the crystal structure of the ERα LBD bound to a TIF-2 box3 peptide” (⁷⁴⁰KENALLKYLLDK⁷⁵¹; PDB ID: 1GWR)—(SEQ ID NO: 21).

ESRRG #08: L3-09—PSEDRGELWRLLSVTERQN (SEQ ID NO: 22)

L3-28—MSVQYPELQRLLMAGSTEL (SEQ ID NO: 23)

L3-80—IPLSGSELSRLLLTEMPEL (SEQ ID NO: 24) (found within text; see ESRRG #09: DAFQLRQLILRGLQDD (SEQ ID NO: 35)—CoRNR-box sequence

Roelens, F.; et al. ‘Subtle side-chain modifications of . . . ER alpha and beta’. J. Med. Chem. 2006, 49(25), 7357-65. PMID: 17149865 (ESRRG #09)

ESRRG #10: SRC1 NR box 4—GPQTPQAQQKSLLQQLLTE (SEQ ID NO: 36)

PGC1a—EAEEPSLLKKLLLAPANTQ (SEQ ID NO: 37)

D22 (RIP140, PGC1, DAX1, SHP)—LPYEGSLLLKLLRAPVEEV (SEQ ID NO: 38) (see Table 4 below)

Table 3-Phage display peptides—“Peptides L3-09, L3-28, and L3-80 were the most effective inhibitors reducing the transcriptional activity of EPPα to basal levels”.

From ‘Abad, M. C., et al. J. Steroid Biochem. Mol. Biol. 2008, 108(1-2), 44-54. PMID: 17964775 (ESRRG #08)—ESRRG #42 was also found through the same PubMed query; —Gaillard, S. et al. ‘Definition of the molecular basis for ERRα-Cofactor interactions’. Mol. Endocrinol. 2007, 21, 62-76.

ESRRG #09: DAFQLRQLILRGLQDD (SEQ ID NO: 35)—CoRNR-box sequence

Roelens, F.; et al. ‘Subtle side-chain modifications of . . . ER alpha and beta’. J. Med. Chem. 2006, 49(25), 7357-65. PMID: 17149865 (ESRRG #09)

ESRRG #10: SRC1 NR box 4—GPQTPQAQQKSLLQQLLTE (SEQ ID NO: 36)

PGC1a—EAEEPSLLKKLLLAPANTQ (SEQ ID NO: 37)

D22 (RIP140, PGC1, DAX1, SHP)—LPYEGSLLLKLLRAPVEEV (SEQ ID NO: 38) (see Table 4 below)

TABLE 3
Shows a copy of a figure from reference ESRRG
#42. Underlined sequences (L3-80, L3-09, and
L3-28) were inferred from context within
manuscript text to be ESRRG candidate ligands.

| | | | |
|---|---|---|---|
| Class | PGC- | RRPCSELLKYLTTNDD | SEQ ID NO: 25 |
| V | 1A13 | | |
| | L3-80 | IPLSGSELSRLLLTEMPEL | SEQ ID NO: 26 |
| | L3-25 | IPVTDSELKRLLLEEVYER | SEQ ID NO: 27 |
| | L3-02 | SGPEFTELYKLLISERDQV | SEQ ID NO: 28 |
| | S3-07 | SELLHLLSHNIDSVSYS | SEQ ID NO: 29 |
| | L3-12 | GVPDGGELRRLLEMDLPEV | SEQ ID NO: 30 |
| | L3-09 | PSEDRGELWRLLSVTERQN | SEQ ID NO: 31 |
| | L3-62 | VSHTPELLAQLLEKPEENL | SEQ ID NO: 32 |
| | L3-07 | NVQMFPELSRLLEKPEENL | SEQ ID NO: 33 |
| | L3-28 | MSVQYPELQRLLMAGSTEL | SEQ ID NO: 34 |

Gowda, et al. Anal Biochem. ‘Development of a coactivator displacement assay for the orphan receptor estrogen-related receptor-gamma using time-resolved fluorescence resonance energy transfer’. 2006 Oct. 1; 357(1):105-15. PMID: 16889744 (ESRRG #10) (Invitrogen Corporation, Drug Discovery Solutions)

“An initial screen of these coregulator peptides bearing the coactivator LXXLL motif, the corepressor LXXI/HIXXXI/L motif, or other interaction motifs from natural coactivator sequences or random phage display peptides indicated that the peptides PGC1alpha, D22, and SRC1-4, known as class III coregulators, interacted most strongly with ERRgamma in the absence of ligand. Unliganded ERRγ also interacted with RIP140 motifs, in agreement with previously reported results”.

TABLE 4
A table of peptides from reference ESRRG #10.
Underlined ligands were inferred from text to
have modulatory effect on/binding affinity
for ESRRG.
Steroid receptor coactivators of the
p160 family

| | |
|---|---|
| SRC1 NR box 1 | KYSQTSHKLVQLLTTTAEQQL |
| | (SEQ ID NO: 39) |
| SRC1 NR box 2 | LTARHKILHRLLQEGSPSD |
| | (SEQ ID NO: 40) |
| SRC1 NR box 3 | ESKDHQLLRYLLDKDEKDL |
| | (SEQ ID NO: 41) |
| SRC1 NR box 4 | GPQTPQAQQKSLLQQLLTE |
| | (SEQ ID NO: 42) |
| SRC2 NR box 1 | DSKGQTKLLQLLTTKSDQM |
| | (SEQ ID NO: 43) |
| SRC2 NR box 2 | LKEKHKILHRLLQDSSSPV |
| | (SEQ ID NO: 44) |
| SRC2 NR box 3 | KKKENALLRYLLDKDDTKD |
| | (SEQ ID NO: 45) |
| SRC3 NR box 1 | ESKGHKKLLQLLTCSSDDR |
| | (SEQ ID NO: 46) |
| SRC3 NR box 2 | LQEKHRILHKLLQNGNSPA |
| | (SEQ ID NO: 47) |
| SRC3 NR box 3 | KKENNALLRYLLDRDDPSD |
| | (SEQ ID NO: 48) |

Other coregulator proteins

| | |
|---|---|
| CBP motif 1 | AASKHKQLSELLRGGSGSS |
| | (SEQ ID NO: 49) |
| TRAP220/DRIP205 motif 1 | KVSQNPILTSLLQITGNGG |
| | (SEQ ID NO: 50) |
| TRAP220/DRIP205 motif 2 | NTKNHPMLMNLLKDNPAQD |
| | (SEQ ID NO: 51) |
| RIP140 motif 6 | SHQKVTLLQLLLGHKNEEN |
| | (SEQ ID NO: 52) |
| RIP140 motif 8 | SFSKNGLLSRLLRQNQDSY |
| | (SEQ ID NO: 53) |

PPAR-specific coactivator proteins

| | |
|---|---|
| PGC1α | EAEEPSLLKKLLLAPANTQ |
| | (SEQ ID NO: 54) |
| PRIP/RAP250 | VTLTSPLLVNLLQSDISAG |
| | (SEQ ID NO: 55) |

Random phage display peptides

| | |
|---|---|
| D22 (RIP140, PGC1, | LPYEGSLLLKLLRAPVEEV |
| DAX1, SHP) | (SEQ ID NO: 56) |
| C33 (TRAP220, RIP140 | HVEMHPLLMGLLMESQWGA |
| | (SEQ ID NO: 57) |
| EAB1 (tamoxifen induced) | SSNHQSSRLIELLSR |
| | (SEQ ID NO: 58) |
| EA2 (tamoxifen induced) | SSKGVLWRMLAEPVSR |
| | (SEQ ID NO: 59) |
| TA1 (tamoxifen induced) | SRTLQLDWGTLYWSR |
| | (SEQ ID NO: 60) |
| TB3 (tamoxifen induced) | SSVASREWWVRELSR |
| | (SEQ ID NO: 61) |

AR-specific peptides

| | |
|---|---|
| AR N terminus (16-34) | SKTYRGAFQNLFQSVREVI |
| | (SEQ ID NO: 62) |
| ARA70 | SRETSEKFKLLFQSYNVND |
| | (SEQ ID NO: 63) |
| D11 FxxLF | VESGSSRFMQLFMANDLLT |
| (GRIP1, SRC1, AIB1) | (SEQ ID NO: 64) |

Corepressor peptides

| | |
|---|---|
| SMRT ID1 | GHQRVVTLAQHISEVITQDYTRH |
| | (SEQ ID NO: 65) |
| SMRT ID2 | HASTNMGLEAIIRKALMGKYDQW |
| | (SEQ ID NO: 66) |
| NCoR ID2 | NLGLEDIIRKALMG |
| | (SEQ ID NO: 67) |

ESRRG #11: A⁶³SHKQLSELLRGG⁷⁵ (CBP LXXLL Motif) (SEQ ID NO: 68)

Klein, F. A.; et al. “Biochemical and NMR mapping of the interface between CREB-binding protein and ligand binding domains of nuclear receptor: beyond the LXXLL motif”. J. Biol. Chem. 2005, 280(7), 2682-92. PMID: 15542861

“The antagonist 4-hydroxytamoxifen induces a conformation of ERR-LBD in which helix H12 is dissociated from the core of the domain (46). Abolition of the interaction of CBP₃₁₋₉₀ with ERRγ LBD by 4-hydroxytamoxifen suggests that LXXLL motif I of CBP contacts NR LBDs in a canonical manner and interacts with helix H12 in an agonist conformation. The hydrophobic cleft formed by this helix was identified previously as the site of interaction on PPARγ LBD by NMR studies using the peptide A⁶³SHKQLSELLRGG⁷⁵ (SEQ ID NO: 68) from CBP”.

PGC-1α-(205-216): RPCSELLKYLTT (SEQ ID NO: [[14]]72); (Same as in entry ESRRG #07).

ESRRG #12: SRC-1-(682-697): SLTARHKILHRLLQEG (SEQ ID NO: 69) (Same as SRC-1 NR Box 2 in Entry 11).

Kallen, J.; et al. ‘Evidence for ligand-independent transcriptional activation of the hERRalpha: crystal structure of ERRalpha LBD in complex with PPAR coactivator-1 alpha’. J. Biol. Chem. 2004, 279(47), 49330-7. PMID: 15337744

“PGC-1α peptide containing the L3 site (Neosystem; sequence: RPASELLKYLTT (SEQ ID NO: 14), i.e. amino acids 205-216 of PGC-1α with the mutation C207A) was added to the protein solution”.

ESRRG #13: RHKILHRLLQEGSPS (SEQ ID NO: 70)—SRC-1 Coactivator Peptide

Greschik, H.; et al. ‘Structural basis for the deactivation of the ERRG by DES or 4-HT and determinants of selectivity’. J. Biol. Chem. 2004, 279(32), 33639-46. PMID: 15161930.

Greschik, H.; et al. ‘Structural and functional evidence for ligand-independent transcriptional activation by the ERR3’. Mol. Cell. 2002, 9(2), 303-13. PMID: 11864604

“New crystals of the ERRα apoLBD grew in the presence of a 3-fold molar excess of a SRC-1 coactivator peptide (⁶⁸⁶RHKILHRLLQEGSPS⁷⁰⁰)” (SEQ ID NO: 70).

ESRRG #14: “SLTERHKILHRLLQEGSPSDI”—(SEQ ID NO: 71) SRC-1.2 (SRC-1 NR Box 2).

Coward, P.; et al. ‘4-HT binds to and deactivates the ERRG’. PNAS 2001, 98(15), 8880-4.

Query 2: NR3B3+peptide ligand

ESRRG #15: Hentschke, M. et al. ‘Characterization of calmodulin binding to the orphan nuclear receptor ERRgamma’. Biol. Chem. 2003, 384(3), 473-82.

No peptide ligand sequences were found.

Query 3: ESRRG+peptide ligand

ESRRG #16: See Wang, et al. entry ESRRG #1.

ESRRG #17: See Gowda, et al. entry ESRRG #10.

ESRRG #18: See Greschik, et al. entry ESRRG #13.

ESRRG #19: See Hentschke, et al. entry ESRRG #15.

Query 4: Nuclear receptor subfamily 3+peptide ligand

ESRRG #20: RPCSELLKYLTT (SEQ ID NO: 72)

Kallen, J.; et al. ‘Crystal structure of hERR alpha in complex with a synthetic inverse agonist’. J. Biol. Chem. 2007, 282(32), 23231-9.

“As FRET acceptor, 1 μl of an N-terminally Cy5-labeled PGC-1a derived peptide (Cy5-RPCSELLKYLTT, (SEQ ID NO: 72), 50 nM final assay concentration) was added”.

ESRRG #21: RGAFQNLFQSV (SEQ ID NO: 73)—AR 20-30.

KENALLRYLLDK (residues 1-12 of SEQ ID NO: 74)—TIF2-III 740-741 (see FIG. 8).

He, B.; et al. ‘Structural basis for AR interdomain and coactivator interactions suggests a transition in NR activation function dominance’. Mol. Cell 2004, 16(3), 425-38. PMID: 15525515.

“Peptide binding affinities were determined by fluorescence polarization (Stanley, et al., 2003) using 40 M R1881 and 17b-estradiol and 10 nM AR 20-30 (fluorescein-RGAFQNLFQSV) (SEQ ID NO: 73) and TIF2-III 740-751 (fluoroscein-KENALLRYLLDK) (residues 1-12 of SEQ ID NO: 74). GAL4-DNA binding domain and VP16 activation domain fusion peptides were prepared.”

ESRRG #22: No sequence found—but reference to GRIP-I, P300, and DRIP 205.

Harris, J. M.; et al. ‘Characterization of RORalpha coactivator binding interface: a structural basis for ligand independent transcription’. Mol. Endocrinol. 2002, 16(5), 998-1012. PMID: 11981035.

“Previously Lau et al. (21) and Atkins et al. (22) had demonstrated ROR interaction with DRIP205, GRIP-1, and P300 using this technique”.

Query 5: “Retinoic Acid Receptor” and Peptide and Ligand—627 references

Changed query term to Query 6.

Query 6: Retinoic Acid Receptor+Binding+Domain—956 references (manually curated top 60 hits)

ESRRG #23: EKHKILHRLLQDSY (SEQ ID NO: 75)—co-activator peptide.

Chandra V, et al. ‘Structure of the intact PPAR-gamma-RXR-alpha nuclear receptor complex on DNA’. Nature, 2008, Nov. 20; 456(7220(350-6. PMID 19043829.

ESRRG #24: PCFXILP (SEQ ID NO: 76)—Dax-1 co-repressor of LRH.

Sablin, E P, et al. ‘The structure of corepressor Dax-1 bound to its target nuclear receptor LRH-1’. PNAS 2008, 105(47), 18390-5. PMID: 19015525.

“Superimposition of the (Dax-1)₂:LRH-1 complex with the structures of hLRH-1 bound to the GRIP-1 peptide (34, 37) shows that the position and conformation of the RH in the complex are similar to those of the docked regulatory peptide. However, the RH site of mDax-1 does not have the LXXLL motif present in nuclear receptor coactivators. Instead, the RH site includes a sequence motif, 275-PCFXXLP-281 (SEQ ID NO: 339), conserved among all members of the NR0B1 subfamily. Thus, the exposed hydrophobic residues C276, I279, and L280 from the Dax-1 repressor helix substitute for the three Leu residues from the LXXLL motif of the coactivators”.

ESRRG #25: “The sequences used in this study were SRC2 NR box 2 (residues 686-698), CGSGKHKILHRLLQDSS (SEQ ID NO: 77); and the only LXXLL motif on DAX1, termed DAX1-3 (residues 142-154), CGSGQGSILYSLLTSSK (SEQ ID NO: 78).”

Valadares, N F et al. ‘Ligand induced interaction of thyroid hormone receptor beta with its coregulators’. J. Ster. Biochem. 2008, 112, 205-12.

ESRRG #26: SRC3-1 peptide (AENQRGPLESKGHKKLLQLLTSS) (SEQ ID NO: 79).

Kruse S W et al. ‘Identification of COUP-TFII Orphan Nuclear Receptor as a Retinoic Acid-Activated Receptor’, PLoS Biol. 2008, 6(9), 227.

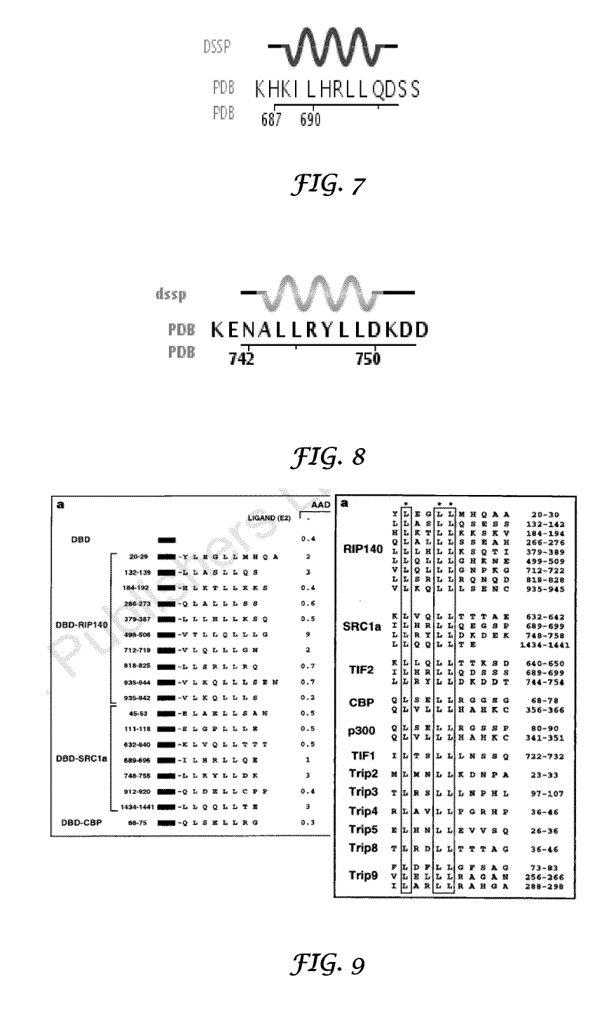

ESRRG #27: KHKILHRLLQDSS (SEQ ID NO: 80)—Nuclear receptor coactivator 2 peptide (PDB 2plt, 2plu, 2plv)—Also see entry ESRRG #3 and FIG. 7.

Nahoum V., et al. ‘Nuclear receptor ligand binding domains: reduction of helix H12 dynamics to favor crystallization’. Acta Cryst. 2008, F64, 614-16. PMID: 18607089

ESRRG #28: No sequence found in text—reference to AA 71-105 of FOXP3. (can be obtained through UniProt or other protein sequence database).

Du, J., et al. ‘Isoform-specific inhibition of RORα-mediated transcriptional activation by Human FOXP3’. J. Immunol. 2008, 180(7), 4785-92. PMID: 18354202.

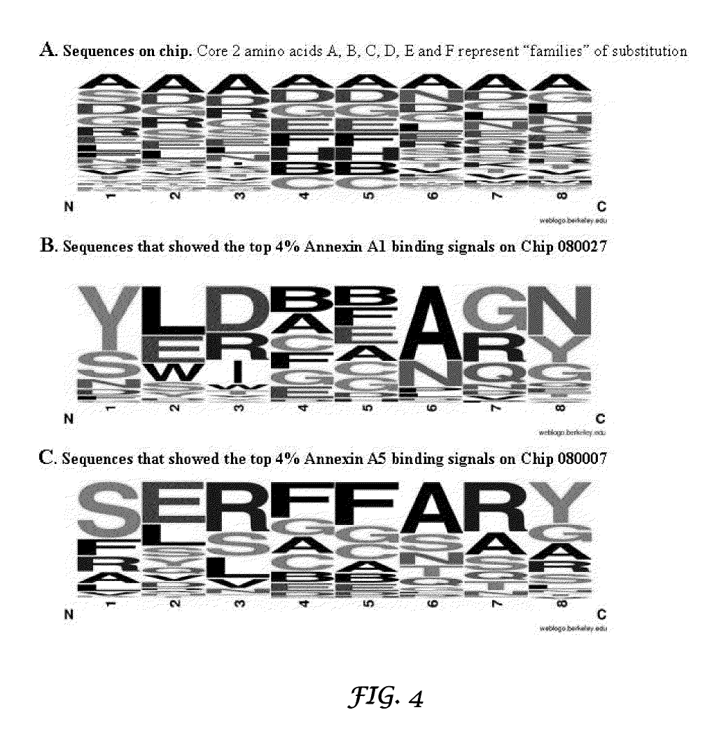

“To map the region in FOXP3 that is responsible for the interaction with RORα, a yeast 2-hybrid test was used”. . . . “As shown in FIG. 4A, only the bait fusion proteins containing the sequences between amino acids 71 and 105, encoded by exon 2, interacted with ROR”.

ESRRG #29: SMRT (a.k.a. NCoR2) and NCoR Corepressor peptides—No sequences given.

Lammi, J., et al. ‘Corepressor interaction differentiates the permissive and non-permissive retinoid X receptor heterodimers’. ABB 2008, 472(2), 105-12. PMID: 18282463.

ESRRG #30: GRIP1 NR box 2 peptide ⁶⁸⁶KHKILHRLLQDSS⁶⁹⁸ (SEQ ID NO: 81).

Lu, J.; The RXRalpha C-terminus T462 is a NMR sensor for coactivator peptide binding. BBRC 2008, 366(4), 932-7. PMID: 18088598.

“Herein, we report a nuclear magnetic resonance (NMR) study of the RXRα ligand-binding domain complexed with 9-cis-retinoic acid and a glucocorticoid receptor-interacting protein 1 peptide.”

ESRRG #31: SRC1 coactivator peptide.

Ito, M. et al. ‘Ab initio fragment molecular orbital study of molecular interactions between liganded retinoid X receptor and its coactivator; part II: influence of mutations in transcriptional activation function 2 activating domain core on the molecular interactions’. J. Phys. Chem. A. 2008, 112(10), 1986-98. PMID: 18020317.

ESRRG #32: CQQQKPQRRP (PGC1-α regions necessary for binding) (SEQ ID NO: 82).

Kanaya, E. and Jingami, H. ‘The region of CQQQKPQRRP (SEQ ID NO: 82) of PGC-1α interacts with the DNA-binding complex of FXR/RXRα’. BBRC, 2006, 342(3), 734-43. PMID: 16494845.

“The results obtained using truncated PGC-1α proteins suggested that two regions are necessary for PGC-1α to interact with the DNA-binding complex of RXRα/FXR. One is the region of the second leucine-rich motif, and the other is that of the amino acid sequence CQQQKPQRRP (SEQ ID NO: 82), present between the second and third leucine-rich motifs”.

ESRRG #33: LTKTNPILYYMLQK (SEQ ID NO: 83)—Derived from RIP40; PMID: 12549917.

“Interestingly, while RIP140 contains nine copies of the LXXLL interaction motif, its ligand-enhanced interaction with holoreceptor appeared to be attributed to a novel peptide (LTKTNPILYYMLQK) (SEQ ID NO: 83) found in its carboxyl-terminal region (amino acids 1063-1076)”.

Query 7: Estrogen Receptor+Peptide+Ligand (PubMed Central)

ESRRG #34: LPALDPTKRWFFETK (SEQ ID NO: 84)—Phage display peptide with Estrogen-like activity. Venkatesh, N. A synthetic peptide with estrogen-like activity derived from a phage-display peptide library. Peptides 2002, 23(3), 573-80. PMID: 11836009.

ESRRG #35: Several peptides from Phage Display—see image below from literature text. Highlighted sequences were inferred from context within text to be candidate ligands for ESRRG.

Hall, J. M. et al. ‘Development of peptide antagonists that target estrogen receptor beta-coactivator interactions’. Mol. Endocrinol. 2000, 14(12), 2010-23. PMID: 11117531 (also see PMID 11117532, 10567548).

TABLE 5
Comparison of the amino acid sequences of the ERβ-interacting peptides.

| | | −3−2−1 | LXXLL | +1+2+3 | |
|---|---|---|---|---|---|
| | Hormone-independent | | | | |
| ERβ-selective peptides | Peptide EBIP-37 | TGGGVSL | LLHLL | NTEQGES | SEQ ID NO: 85 |
| | Peptide EBIP-41 | RRDDFPL | LISLL | KDGALSQ | SEQ ID NO: 86 |
| | Peptide EBIP-44 | YGLKMSL | LESLL | REDISTV | SEQ ID NO: 87 |
| | Peptide EBIP-45 | MSYDMLS | LYPLL | TNSLLEV | SEQ ID NO: 88 |
| | Peptide EBIP-51 | FPAEFPL | LTYLL | ERQGMDE | SEQ ID NO: 89 |
| | Peptide EBIP-96 | VESEFPY | LLSLL | GEVSPQP | SEQ ID NO: 90 |
| | Hormone-dependent | | | | |
| | Peptide EBIP-49 | VSSEGRL | LIDLL | VGQQSE | SEQ ID NO: 91 |
| | Peptide EBIP-53 | DTPQSPL | LWGLL | SSDRVEG | SEQ ID NO: 92 |
| | Peptide EBIP-60 | GGTQDGY | LWSLL | TGMPEVS | SEQ ID NO: 93 |
| | Peptide EBIP-66 | SLPEEGF | LMKLL | TLEGDAE | SEQ ID NO: 94 |
| | Peptide EBIP-70 | VMGNNPI | LVSLL | EEPSEEP | SEQ ID NO: 95 |
| | Peptide EBIP-76 | VLVEHPI | LGGLL | STRVDSS | SEQ ID NO: 96 |
| | Peptide EBIP-87 | QTPL | LEQLL | TEHIQQG | SEQ ID NO: 97 |
| ERβ-specific peptides | Peptide EBIP-56 | GSWQDSL | LLQLL | NRTELMA | SEQ ID NO: 98 |
| | Peptide EBIP-92 | SVWPGPE | LLKLL | SGTSVAE | SEQ ID NO: 99 |

The ERβ-selective peptides were divided into two classes: hormone-independent and hormone-dependent. Also shown are the sequences of the ERβ-specific peptides EBIP-56 and EBIP-92, which constitute a third class of ERβ-interacting peptides.
The conserved tryptophan at position −5 relative to the LXXLL motif in these two sequences is shown in bold.

ESRRG #36: VSWFFE (SEQ ID NO: 100), VSWFFED (SEQ ID NO: 101)—Derived from mutation analysis of PhD peptide LPALDPTKRWFFETK (SEQ ID NO: 84) (see entry ESRRG #34).

Kasher, et al. ‘Design, synthesis, and evaluation of peptides with estrogen like activity’. Peptide Science 2004, 76(5), 404-420. PMID: 15468062.

“VSWFFE (EMP-1) (SEQ ID NO: 100) and VSWFFED (EMP-2) (SEQ ID NO: 101) (EMP: estrogen-mimetic peptide), bind mAb-E2 with high affinity (IC50 of 6 and 30 nM, respectively), recognize ERs with increased affinity (IC50 of 100 μM for ERα, and 100-250 μM for ERβ), and possess estrogenic activity in vivo”.

ESRRG #37: KClLCRLLQ—(H-Lys-cyclo-(d-Cys-Ile-Leu-Cys)-Arg-Leu-Leu,Gln-NH₂) (SEQ ID NO: 102).

Leduc, A. M.; et al. ‘Helix-stabilized cyclic peptides as selective inhibitors of steroid receptor-coactivator interactions’. (see entry ESRRG 5).

ESRRG #38: NIFSEVRVYN (SEQ ID NO: 103) AA 795-804 of DRIP150 coactivator protein.

Lee, J and Safe, S. ‘Coactivation of estrogen receptor alpha (ER alpha)/Sp1 by vitamin D receptor interacting protein 150 (DRIP150)’. ABB 2007, 461(2), 200-210. PMID: 17306756.

“Deletion analysis of DRIP150 demonstrates that coactivation requires an alpha-helical NIFSEVRVYN (SEQ ID NO: 103) (amino acids 795-804) motif within 23 amino acid sequence (789-811) in the central region of DRIP150 and similar results were obtained for coactivation of ERalpha by DRIP150”.

ESRRG #39: VLWKLLKVV (SEQ ID NO: 104)—Sequence from R-MGMT.

Teo, et al. ‘The modified human DNA repair enzyme O(6)-methylguanine-DNA methyltransferase is a negative regulator of estrogen receptor-mediated transcription upon alkylation DNA damage’. Mol. Cell Biol. 2001, 21(20), 7105-14. PMID: 11564893.

“R-MGMT, which adopts an altered conformation, utilizes its exposed VLWKLLKVV (SEQ ID NO: 104) peptide domain (codons 98 to 106) to bind ER. This binding blocks ER from association with the LXXLL motif of its coactivator, steroid receptor coactivator-1, and thus represses ER effectively from carrying out transcription that regulates cell growth”.

Query 8: Estrogen Receptor and Peptide and Ligand (125 PMC articles)

ESRRG #40: SGSGLTSRDFGSWYA (SEQ ID NO: 105), EKHKILHRLLQDS (SEQ ID NO: 106).

Kong, et al. ‘Delineation of a unique P-P interaction site on the surface of the ER’. PNAS, 2005, 3593-98. PMID: 15728727, PDBID—2BJ4-A.

“αII Peptide Motif Interacts Specifically with Liganded ERαLBD. Previous reports of αII binding to ERα showed that the interaction occurs within the C-terminal LBD region between residues 282 and 535”.

Query 9: Retionoid X Receptor and Peptide and Ligand (58 PMC articles)

ESRRG #41: Multiple peptides—co-activators, co-repressors, and interacting protein sequences.

Folkertsma, S.; et al. ‘The use of in vitro peptide binding profiles and in silico ligand-receptor interaction profiles to describe ligand induced conformations of the RXRa LBD’. Mol. Endocrinol. 2007 21(1), 30-48. PMID: 17038419.

“FIG. 1, A and B, shows that the remaining 13 peptides equally bind to RXRα with and without 9-cis RA. The estimated K_{d} app values of the dose-response curves in the absence and presence of 9-cis RA are nearly equal (152 nM in the absence and 155 nM in the presence of compound), which indicates that there is no effect on the peptide binding by 9-cis RA.”.

ESRRG #42: ⁶⁸⁶RHKILHRLLQEGSPS⁷⁰⁰ (SEQ ID NO: 107)—SRC-1 Coactivator.

Groot, A. D.; et al. ‘Crystal structure of a novel tetrameric complex of agonist-bound LBD of Biomphalaria glabrata RXR’. JMB, 2005, 354, 841-53. PMID: 16274693.

Query 10: Progesterone Receptor and Peptide and Ligand (40 PMC articles, no hits)

Query 11: RXR+peptide+ligand

ESRRG #43: Crystal Structure Of Human Lrh-1 Bound With Tif-2 Peptide And Phosphatidylglycerol. (Expression_system: Escherichia Coli; Expression_system_common: Bacteria; Mol_id: 2; Synthetic: Yes; Other_details: The Peptide Was Chemically Synthesized. The Sequence Of The Peptide Is Naturally Found In Homo Sapiens. (Human)

Query 12: “ESRRG-binding domain”

ESRRG #44: Gowda K, et al. Anal Biochem. 2006 Oct. 1; 357(1):105-15. (Epub 2006 Jul. 10. PMID: 16889744) ‘Development of a coactivator displacement assay for the orphan receptor estrogen-related receptor-gamma using time-resolved fluorescence resonance energy transfer’. Invitrogen Corporation, Drug Discovery Solutions, Madison, Wis. 53719, USA.

### B)

| SWISS-PROT | Peptide | | |
|---|---|---|---|
| ID | Name | Position | Sequence |
| PRGC1_HUMAN | PGC_1 | 130-155 | N-Biotinyl- DGTPPPQEAEEPSLLKKLLLAPANTQ -C |
| | | | SEQ ID NO: 108 |
| SHP_HUMAN | SHP_1 | 9-33 | N-Biotinyl- CPCQGAASRPAILYALLSSSLKAVP -C |
| | | | SEQ ID NO: 109 |
| NCOA3_HUMAN | SRC3_3 | 724-748 | N-Biotinyl- QEQLSPKKKENNALLRYLLDRDDPS -C |
| | | | SEQ ID NO: 110 |
| NRIP1_HUMAN | RIP140_8 | 805-831 | N-Biotinyl- PVSPQDFSFSKNGLLSRLLRQNQDSYL -C |
| | | | SEQ ID NO: 111 |
| NCOA2_HUMAN | SRC2_3 | 732-756 | N-Biotinyl- QEPVSPKKKENALLRYLLDKDDTKD -C |
| | | | SEQ ID NO: 112 |
| NCOA1_HUMAN | SRC1a_4 | 1421-1441 | N-Biotinyl- TSGPQTPQAQQKSLLQQLLTE -C |
| | | | SEQ ID NO: 113 |
| NRIP1_HUMAN | RIP140_9 | 922-946 | N-Biotinyl- EHRSWARESKSFNVLKQLLLSENCV -C |
| | | | SEQ ID NO: 114 |
| DAX1_HUMAN | DAX_3_cys | 134-159 | N-Biotinyl- FCGEDHPRQGSILYSLLTSSKQTHVA -C |
| | | | SEQ ID NO: 115 |
| NRIP1_HUMAN | RIP140_5 | 366-390 | N-Biotinyl- LERNNIKQAANNSLLLHLLKSQTIP -C |
| | | | SEQ ID NO: 116 |
| DAX1_HUMAN | DAX_3 | 136-159 | N-Biotinyl- GEDHPRQGSILYSLLTSSKQTHVA -C |
| | | | SEQ ID NO: 117 |
| NCOA1_HUMAN | SRC1_2 | 676-700 | N-Biotinyl- CPSSHSSLTERHKILHRLLQEGSPS -C |
| | | | SEQ ID NO: 118 |
| NCOA3_HUMAN | SRC3_2 | 671-695 | N-Biotinyl- SNMHGSLLQEKHRILHKLLQNGNSP -C |
| | | | SEQ ID NO: 119 |
| NRIP1_HUMAN | RIP140_3 | 172-196 | N-Biotinyl- EKDLRCYGVASSHLKTLLKKSKVKD -C |
| | | | SEQ ID NO: 120 |

“The estrogen-related receptor-gamma (ERRgamma) is a constitutively active orphan receptor that belongs to the nuclear receptor superfamily and is most closely related to the estrogen receptors. Although its physiological ligand is unknown, ERRgamma has been shown to interact with synthetic estrogenic compounds such as 4-hydroxytamoxifen (4-OHT), tamoxifen, and diethylstilbestrol (DES). To assess how coregulator proteins interact with ERRgamma in response to ligand, an in vitro interaction methodology using time-resolved fluorescence resonance energy transfer (TR-FRET) was developed using glutathione 5-transferase (GST)-tagged ERRgamma ligand-binding domain (LBD), a terbium-labeled anti-GST antibody, a fluorescein-labeled peptide containing sequences derived from coregulator proteins, and various ligands. An initial screen of these coregulator peptides bearing the coactivator LXXLL motif, the corepressor LXXI/HIXXXI/L motif, or other interaction motifs from natural coactivator sequences or random phage display peptides indicated that the peptides PGC1alpha, D22, and SRC1-4, known as class III coregulators, interacted most strongly with ERRgamma in the absence of ligand. Given its assay window and biological relevance in energy metabolism and obesity, further studies were conducted with PGC1alpha. Fluorescein-labeled PGC1alpha peptide was displaced from the ERRgamma LBD in the presence of increasing concentrations of 4-OHT and tamoxifen, but DES was less effective in PGC1alpha displacement. The statistical parameter Z′ factor that measures the robustness of the assay was greater than 0.8 for displacement of PGC1alpha from ERRgamma LBD in the presence of saturating 4-OHT over an assay incubation time of 1-6 h, indicating an excellent assay. These findings also suggest that binding of 4-OHT, tamoxifen, or DES to ERRgamma results in differential affinity of coregulators for ERRgamma due to unique ligand-induced conformations”.

ESRRG #45: Wang L, et al. ‘X-ray crystal structures of the estrogen-related receptor-gamma ligand binding domain in three functional states reveal the molecular basis of small molecule regulation’. J Biol Chem. 2006 Dec. 8; 281(49):37773-81. Epub 2006 Sep. 21. (PMID: 16990259) (Discovery Research, GlaxoSmithKline, Research Triangle Park, N.C. 27909, USA).

“X-ray crystal structures of the ligand binding domain (LBD) of the estrogen-related receptor-gamma (ERRgamma) were determined that describe this receptor in three distinct states: unliganded, inverse agonist bound, and agonist bound. Two structures were solved for the unliganded state, the ERRgamma LBD alone, and in complex with a coregulator peptide representing a portion of receptor interacting protein 140 (RIP140). No significant differences were seen between these structures that both exhibited the conformation of ERRgamma seen in studies with other coactivators. Two structures were obtained describing the inverse agonist-bound state, the ERRgamma LBD with 4-hydroxytamoxifen (4-OHT), and the ERRgamma LBD with 4-OHT and a peptide representing a portion of the silencing mediator of retinoid and thyroid hormone action protein (SMRT). The 4-OHT structure was similar to other reported inverse agonist bound structures, showing reorientation of phenylalanine 435 and a displacement of the AF-2 helix relative to the unliganded structures with little other rearrangement occurring. No significant changes to the LBD appear to be induced by peptide binding with the addition of the SMRT peptide to the ERRgamma plus 4-OHT complex. The observed agonist-bound state contains the ERRgamma LBD, a ligand (GSK4716), and the RIP140 peptide and reveals an unexpected rearrangement of the phenol-binding residues. Thermal stability studies show that agonist binding leads to global stabilization of the ligand binding domain. In contrast to the conventional mechanism of nuclear receptor ligand activation, activation of ERRgamma by GSK4716 does not appear to involve a major rearrangement or significant stabilization of the C-terminal helix”. (See Queries 1-5).

ESRRG #46: ‘A signature motif in transcriptional co-activators mediates binding to nuclear receptors’. David M. Henry, et al. Nature Letters 1997, 387, 7330736. (see FIG. 9)

ESRRG #47: Zhou D, et al. ‘PNRC: a proline-rich nuclear receptor coregulatory protein that modulates transcriptional activation of multiple nuclear receptors including orphan receptors SF1 (steroidogenic factor 1) and ERRalpha1 (estrogen related receptor alpha-1)’. Mol Endocrinol. 2000 July; 14(7):986-98. PMID: 10894149.

“PNRC (proline-rich nuclear receptor coregulatory protein) was identified using bovine SF1 (steroidogenic factor 1) as the bait in a yeast two-hybrid screening of a human mammary gland cDNA expression library. PNRC is unique in that it has a molecular mass of 35 kDa, significantly smaller than most of the coregulatory proteins reported so far, and it is proline-rich. PNRC's nuclear localization was demonstrated by immunofluorescence and Western blot analyses. In the yeast two-hybrid assays, PNRC interacted with the orphan receptors SF1 and ERRalpha1 in a ligand-independent manner. PNRC was also found to interact with the ligand-binding domains of all the nuclear receptors tested including estrogen receptor (ER), androgen receptor (AR), glucocorticoid receptor (GR), progesterone receptor (PR), thyroid hormone receptor (TR), retinoic acid receptor (RAR), and retinoid X receptor (RXR) in a ligand-dependent manner. Functional AF2 domain is required for nuclear receptors to bind to PNRC. Furthermore, in vitro glutathione-5-transferase pull-down assay was performed to demonstrate a direct contact between PNRC and nuclear receptors such as SF1. Communoprecipitation experiment using Hela cells that express PNRC and ER was performed to confirm the interaction of PNRC and nuclear receptors in vivo in a ligand-dependent manner. PNRC was found to function as a coactivator to enhance the transcriptional activation mediated by SF1, ERR1 (estrogen related receptor alpha-1), PR, and TR. By examining a series of deletion mutants of PNRC using the yeast two-hybrid assay, a 23-amino acid (aa) sequence in the carboxy-terminal region, aa 278-300, was shown to be critical and sufficient for the interaction with nuclear receptors. This region is proline rich and contains a SH3-binding motif, S-D-P-P-S-P-S (SEQ ID NO: 340). Results from the mutagenesis study demonstrated that the two conserved proline (P) residues in this motif are crucial for PNRC to interact with the nuclear receptors. The exact 23-amino acid sequence was also found in another protein isolated from the same yeast two-hybrid screening study. These two proteins belong to a new family of nuclear receptor coregulatory proteins”.

Candidate Ligand Browse Table

A list of candidate ligand sequences for ESRRG proteins is shown below. A compendium of identified peptide sequences known to bind to/interact with ESRRG or its sequence homologs is given below in Table 6. Table 6 lists each of the candidate sequences, the source of the sequence and the protein with which it interacts, as well as a reference to the literature source from which it was derived. All the listed information, and the context in literature from which peptide was derived (e.g., Abstract, Material and Methods, etc.) can be obtained currently through the computerized HALO/Gen web portal.

TABLE 6
A list of candidate ligand sequences for ESRRG proteins.

| | | | ESRRG |
|---|---|---|---|
| Candidate Sequences | Source | | Ref # |
| | | SEQ ID | |
| | | NO: | |
| LERNNIKQAANNSLLLHLLKSQTIP | RIP40 peptide/ERRα LBD | 2 | 1 |
| RHKILHRLLQEGSPS | Steroid receptor coactivator | 3 | 2, 13, 42 |
| | 1/ERR3 LBD | | |
| KHKILHRLLQDSS | Steroid receptor coactivator | 4 | 3, 27 |
| | 3/ER α | | |
| SGSHKLVQLLTTT | WAY 697/ER α | 5 | 4 |
| TNMGLEAIRKALMGKYDQWEE | SMRT Peptide | 6 | 5, 37 |
| QAQQKSLLQQLLTE | SRC-1 NR box 4 peptide (ER α) | 7 | |
| LTERHKILHRLLQEGSPSD | SRC-1 NR box 2 peptide (ER α) | 8 | |
| cyclo-KDCILCRLLN | PERM-1 | 9 | |
| GGSCPSSHSSLTERH | SRC-1 residues 683-67 (ERR α | 10 | 6 |
| | LBD) | | |
| QQQKPQRRPCSELLKYLTTNDD | PPAR Gamma Coactivator (PGC- | 11 | 7, 43 |
| | 1) Box 3/ERR α-LBD | | |
| KENALLKYLLDK | TIF-2 Box 3 peptide/ER α LBD | 12 | |
| PSEDRGELWRLLSVTERQN | Phage display peptides/ERR α | 22 | 8 |
| MSVQYPELQRLLMAGSTEL | Phage display peptides/ERR α | 23 | |
| IPLSGSELSRLLLTEMPEL | Phage display peptides/ERR α | 24 | |
| DAFQLRQLILRGLQDD | CoRNR-box sequence/ER α/ER α | 35 | 9 |
| GPQTPQAQQKSLLQQLLTE | SRC1 NR box 4/ERR3 | 36 | 10 |
| EAEEPSLLKKLLLAPANTQ | PGC1 α/ERR3 | 37 | |
| LPYEGSLLLKLLRAPVEEV | D22 (RIP140, PGC1, DAX1, | 38 | |
| | SHP)/ERR3 | | |
| SHQKVTLLQLLLGHKNEEN | RIP140 motif 6 | 52 | |
| SFSKNGLLSRLLRQNQDSY | RIP140 motif 8 | 53 | |
| ASHKQLSELLRGG | CBP/ERR α-LBD | 68 | 11 |
| RPCSELLKYLTT | PGC-1 α-(205-216) | 72 | 12 |
| SLTARHKILHRLLQEG | SRC-1 NR Box 2 (682-697) | 69 | |
| SLTERHKILHRLLQEGSPSDI | SRC-1 NR Box 2 | 71 | 14 |
| RPCSELLKYLTT | PGC-1 α derived peptide | 72 | 20 |
| RGAFQNLFQSV | AR 20-30 | 73 | 7, 21 |
| | | Residues | |
| KENALLRYLLDK | TIF2-III 740-741 | 1-12 of | |
| | | 74 | |
| EKHKILHRLLQDSY | co-activator peptide SRC2 | 75 | 23, 40 |
| PCFXILP | Dax-1 co-repressor/LRH | 76 | 24 |
| CGSGKHKILHRLLQDSS | SRC2 NR box 2 (residues 686- | 77 | 25 |
| | 698) | | |
| CGSGQGSILYSLLTSSK | DAX1-3 (residues 142-154)/Thyr. | 78 | |
| | Recep. | | |
| AENQRGPLESKGHKKLLQLLTSS | SRC3-1 peptide/RXR | 79 | 26 |
| No sequence | AA 71-105 of FOXP3 | | 28 |
| No sequence | SMRT (a.k.a. NCoR2) and NCoR | | 29 |
| | Corepressor peptides | | |
| KHKILHRLLQDSS | GRIP1 NR box 2/RXR | 81 | 3, 27, 30 |
| CQQQKPQRRP | PGC1- α/FXR-RXR α | 82 | 32 |
| LTKTNPILYYMLQK | RIP40/RAR and RXR | 83 | 33 |
| LPALDPTKRWFFETK | Ph. D. peptide/ER | 84 | 34 |
| VSWFFE, VSWFFED | Ph.D- EMP: estrogen-mimetic | 100, 101 | 36 |
| | peptide | | |
| NIFSEVRVYN | DRIP150 Coactivator/ER α | 103 | 38 |
| VLWKLLKVV | R-MGMT/ER | 104 | 39 |
| SGSGLTSRDFGSWYA | ER alpha | 105 | 40 |

Step 3: Sequence Selection

Objective: To parse candidate ligand table to remove redundant, closely similar, or entrained (shorter sequence within larger candidate peptide ligand) sequences in order to focus the ligand space (see Table 7).

In a first step, candidate peptide ligands for ESRRG were subjected to a multiple sequence alignment algorithm (ClustalW2). Stringent alignment parameters were selected for this: penalty for Gap Extension=10.0, Gap Open=100, and End Gaps=“No”; in order to prevent gaps during alignment. All other parameters were set at default. Sequence alignment was then taken into consideration as candidate ligands were downselected for screening. All sequences contained within larger sequences (entrained), or highly similar (pairwise similarity score of >70%, incremental scale) to other sequences were removed. Longer sequences were retained in each case. Cysteine containing sequences were either removed or substituted by Alanine. Sequences longer than 8 were only considered, shorter sequences were culled.

TABLE 7
ClustalW2: Multiple alignment of
ESRRG candidate ligands.

| | |
|---|---|
| seq6 --------QAQQKSLLQQLLTE-------14 | SEQ ID NO:121 |
| 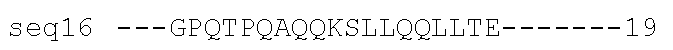 | SEQ ID NO:122 |
| 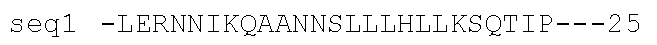 | SEQ ID NO:123 |
| seq4 ---------S-GSHKLVQLLTTT------13 | SEQ ID NO:124 |
| 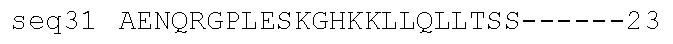 | SEQ ID NO:125 |
| 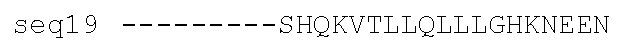 | SEQ ID NO:126 |
| 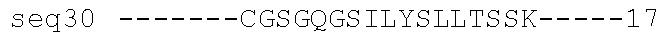 | SEQ ID NO:127 |
| seq11 ----------KENALLKYLLDK-------12 | SEQ ID NO:128 |
| seq27 ----------KENALLKYLLDK-------12 | SEQ ID NO:129 |
| 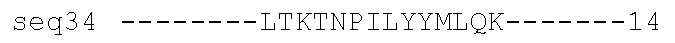 | SEQ ID NO:130 |
| 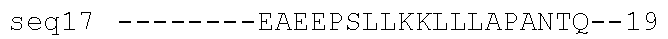 | SEQ ID NO:131 |
| 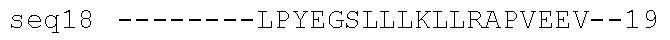 | SEQ ID NO:132 |
| 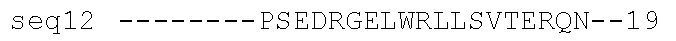 | SEQ ID NO:133 |
| 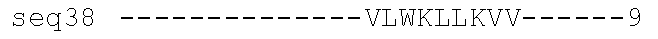 | SEQ ID NO:134 |
| seq2 -----------RHKILHRLLQEGSPS---15 | SEQ ID NO:135 |
| seq7 --------LTERHKILHRLLQEGSPSD--19 | SEQ ID NO:136 |
| 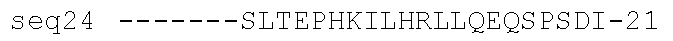 | SEQ ID NO:137 |
| seq23 -------SLTARHKILHRLLQEG------16 | SEQ ID NO:138 |
| seq3 -----------KHKILHRLLQDSS-----13 | SEQ ID NO:139 |
| seq29 -------CGSGKHKILHRLLQDSS-----17 | SEQ ID NO:140 |
| seq32 -----------KHKILHRLLQDSS-----13 | SEQ ID NO:141 |
| seq28 ----------EKHKILHRLLQDSY-----14 | SEQ ID NO:142 |
| 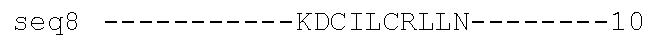 | SEQ ID NO:143 |
| 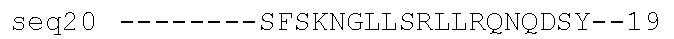 | SEQ ID NO:144 |
| 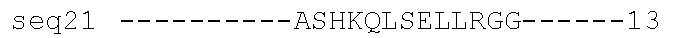 | SEQ ID NO:145 |
| 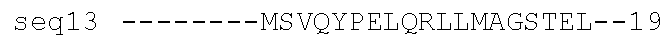 | SEQ ID NO:146 |
| 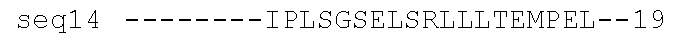 | SEQ ID NO:147 |
| 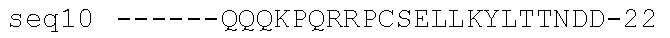 | SEQ ID NO:148 |
| seq33 -----CQQQKPQRRP--------------10 | SEQ ID NO:149 |
| seq22 -------------RPCSELLKYLTT----12 | SEQ ID NO:150 |
| seq25 -------------RPCSELLKYLTT----12 | SEQ ID NO:151 |
| 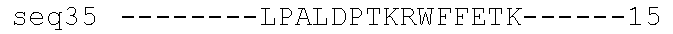 | SEQ ID NO:152 |
| seq36 ---------------VSWFFED-------7 | SEQ ID NO:153 |
| 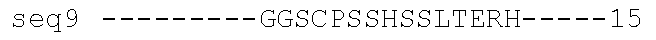 | SEQ ID NO:154 |
| 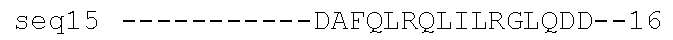 | SEQ ID NO:155 |
| 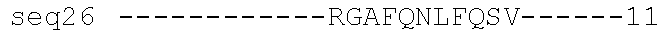 | SEQ ID NO:156 |
| 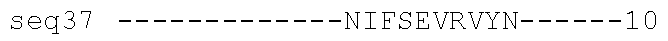 | SEQ ID NO:158 |
| 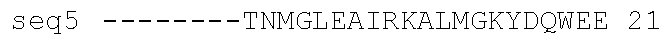 | SEQ ID NO:159 |

Candidate Ligand Down-Selection from ClustalW2 Input 1

TABLE 8
One approach to candidate ligand selection for peptide micro-array generation is
described. Emboldened sequences were selected; crossed-out sequences were removed. When
entrained sequences were found, the longer sequence was retained. When two or more highly similar
candidate peptides were found, the longest of the similar sequence was used. For example, Seq 2
(entry 2) is same as (=) and contained within Seq 7 and Seq 24 and related to (~) Seq 23. Hence, Seq
2 was not considered for sequence expansion and arraying. Similarly, Seq 7 is present within Seq 24
(designated >24<); hence Seq 24 was selected whereas Seq 7 was rejected. Other symbolic
descriptors of correlations are listed at the bottom of the table. The SEQ ID NOS. of the selected
sequences are identified in parenthesis.

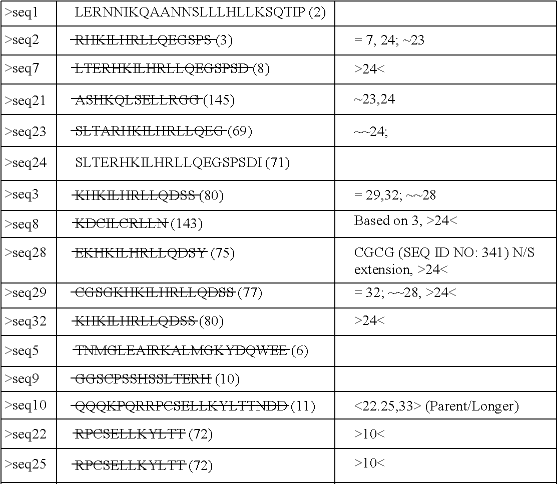

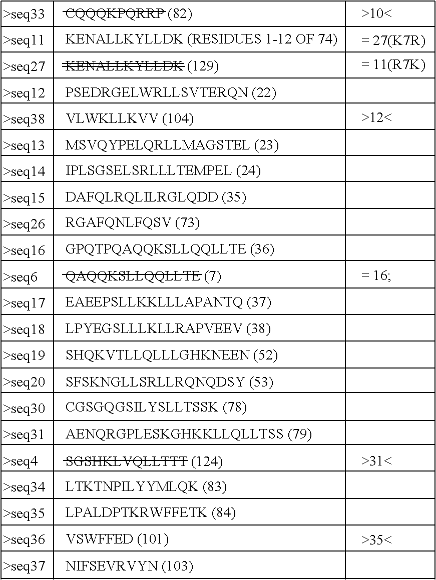

>XX< = XX is parent (longer) of selected sequence
<YY> = YY (or conservative substituent) is contained within selected sequence (aka parent XX)
= implies 100% similarity;
~~ indicates very similar,
~ indicates related to Longer sequence preferentially selected over shorter one; conserved substitutions (BLOSUM62) are ignored.

Step 4: Candidate Ligand Walking and Sequence Expansion

Ligands of longer than 8 AAs were ligand (or frame) walked 1 amino acid at a time to create a library of candidate 8-mer parent ligands. Sequence space was then expanded in the manner described below to create an 8-mer superlibrary of parent peptides.

The BLOSUM62 matrix was divided into Tiers based, with Tier 1 comprising “most conserved” (lowest penalty) and Tier 3 comprising “least conserved” (highest penalty) mutations observed in nature. Sequences for microarray production for the primary stage (Iteration 1) of screening for protein binding were created by permuting AA positions 4 and/or 5 only (i.e., core of sequence) of parent 8-mer peptides using Tier 1 and Tier 3 substitutions. This allows the systematic variation of the sequence in order to identify the most important motif within the larger candidate ligand sequence that is needed for binding the target protein. All peptides on the array were grown from the C→N terminal, with the exposed N-terminal amino acid masked by an acetyl group to prevent folding of the sequence and enable full presentation of the peptide to its target protein.

Screening was carried out typically by hybridization of the protein (1 μg/mL) against the array for 1 h at 4° C.; detection of bound protein was carried out using a fluorescently labeled anti-ESRRG mAb. (Direct labeling of ESRRG and alternative detection strategies are equally valid).

Highest binders (based on relative fluorescence signal) were selected and iteratively sequence expanded and arrayed (Iteration 2); and ESRRG binding sequences were selected as above. For iterative arrays, the core positions (AA 4 and 5) were kept unchanged while the outer amino acids (2, 3, 6, and/or 7) were permuted using Tier 3 substitutions.

Step 5: Screening and Assays

Daughter candidate ligands and their new Tier 3 and/or Tier 1 based combinations were synthesized in situ on an addressable peptide microarray and screened against native human ESRRG protein in order to determine sequences that bind to this protein. Bound protein was detected by a fluorescently labeled anti-ESRRG mAb. Results are obtained in fluorescence intensity units which were used to rank the peptide ligands. Excess (100×) competing biological matrices (human serum, HSA/BSA or bacterial protein extract) were used as backgrounds to select for specific ligands to the protein. Other competing proteins can be added to background for specificity selection and directed evolution of ligands.

Iteration 1: Results of Screening

The top 50 strongest binding ligands (ranked by Log₂ [Signal/Background] and fluorescence signal) from Iteration 1 screening against ESRRG are listed in Table 9. All peptides were clearly distinct from their parent 8-mer and showed significantly higher signal from protein binding than their respective parent sequences.

TABLE 9
A list of the top 50 strongest-
binding ligands to ESRRG from the
primary screen (Iteration 1).

| | | Sample | | log2 |
|---|---|---|---|---|
| Peptide | SEQ | A | Sample B | (Sample |
| Sequence | ID | (Bkgd) | (Probe) | B/ |
| N→C | NO: | Signal | Signal | Sample A) |
| NSLLQHLL | 160 | 2.42 | 9,739.76 | 11.97 |
| NSLKKHLL | 161 | 5.04 | 9,219.14 | 10.84 |
| ANNSQLLH | 162 | 1.00 | 955.18 | 9.90 |
| IKQNHNNS | 163 | 1.00 | 793.75 | 9.63 |
| IKQDHNNS | 164 | 1.00 | 701.08 | 9.45 |
| ANNIQLLH | 165 | 2.12 | 805.56 | 8.57 |
| NSLLKHLL | 166 | 73.81 | 14,810.16 | 7.65 |
| NIKVNANN | 167 | 3.04 | 598.87 | 7.62 |
| AANYLLLL | 168 | 22.22 | 3,763.81 | 7.40 |
| ANNSSLLH | 169 | 7.39 | 1,153.13 | 7.29 |
| AANYVLLL | 170 | 14.74 | 2,199.01 | 7.22 |
| AANYILLL | 171 | 13.78 | 2,012.02 | 7.19 |
| AANNYLLL | 172 | 31.22 | 3,436.10 | 6.78 |
| NIKVHANN | 173 | 9.00 | 862.67 | 6.58 |
| ANNSLLLH | 174 | 14.53 | 1,321.14 | 6.51 |
| LHLKGSQT | 175 | 6.97 | 622.22 | 6.48 |
| KSLLQQLL | 176 | 88.50 | 7,385.40 | 6.38 |
| NSLRSHLL | 177 | 20.67 | 1,673.50 | 6.34 |
| ANNILLLH | 178 | 13.51 | 1,093.06 | 6.34 |
| AANMSLLL | 179 | 16.99 | 1,217.01 | 6.16 |
| NSLRQHLL | 180 | 47.85 | 3,242.55 | 6.08 |
| ANNSWLLH | 181 | 20.06 | 1,259.63 | 5.97 |
| AANMYLLL | 182 | 64.73 | 4,011.85 | 5.95 |
| AIRVHLMG | 183 | 11.78 | 689.75 | 5.87 |
| ANNLSLLH | 184 | 21.54 | 1,220.01 | 5.82 |
| LLHLQKSQ | 185 | 14.95 | 745.24 | 5.64 |
| AIRGHLMG | 186 | 18.88 | 934.65 | 5.63 |
| ANNSRLLH | 187 | 31.74 | 1,480.35 | 5.54 |
| AANMLLLL | 188 | 25.96 | 1,191.86 | 5.52 |
| AIRGALMG | 189 | 19.03 | 851.06 | 5.48 |
| ANNSKLLH | 190 | 23.60 | 1,009.30 | 5.42 |
| LLHSSKSQ | 191 | 19.42 | 819.16 | 5.40 |
| AANMFLLL | 192 | 42.26 | 1,752.81 | 5.37 |
| NSLRKHLL | 193 | 20.79 | 856.14 | 5.36 |
| NSLSWHLL | 194 | 25.76 | 1,042.20 | 5.34 |
| ANNLWLLH | 195 | 41.92 | 1,606.54 | 5.26 |
| ANNFLLLH | 196 | 46.43 | 1,624.73 | 5.13 |
| RKAIFGKY | 197 | 23.63 | 818.19 | 5.11 |
| AIRLHLMG | 198 | 28.09 | 942.75 | 5.07 |
| ANNIWLLH | 199 | 30.52 | 1,012.72 | 5.05 |
| ANNVKLLH | 200 | 52.38 | 1,718.47 | 5.04 |
| ANNLQLLH | 201 | 47.08 | 1,537.71 | 5.03 |
| LLHKSKSQ | 202 | 30.24 | 962.22 | 4.99 |
| IKQNYNNS | 203 | 23.70 | 740.23 | 4.97 |
| NNSWSLHL | 204 | 23.54 | 728.51 | 4.95 |
| RKALVGKY | 205 | 28.08 | 868.05 | 4.95 |
| ANNVLLLH | 206 | 65.96 | 1,997.75 | 4.92 |
| ANNLKLLH | 207 | 56.94 | 1,672.30 | 4.88 |
| ANNVSLLH | 208 | 62.19 | 1,818.72 | 4.87 |
| LHLRYSQT | 209 | 33.10 | 960.75 | 4.86 |

Iteration 2—Results of Screening

Iterative sequence expansion and arraying of 10 highest binding ligands to ESRRG from Iteration 1 was carried out and the new peptide sequences were synthesized on an array and screened against the protein, as described above. In this step, the central 2 amino acids (core) in peptide sequences were maintained unchanged while outlying amino acids (2, 3, 6, and 7) were permuted (using Tier 1 and Tier 3 substitutions) in a ‘cipher’ or ‘combination’ lock approach. A list of the top 50 ligands from iterative screening is given in Table 10. Further affinity maturation of sequence was observed, with multifold enhancement in binding signal of Iteration 2 sequences (i.e., Granddaughters) compared to their respective parents from Iteration 1 (i.e., Daughters) on the same array.

TABLE 10
Results of iterative sequence expansion and
screening of top 10 strongest binding
ligands to ESRRG from the primary screen
(Iteration 1).

| Peptide | SEQ | Sample A | Sample B | log2 |
|---|---|---|---|---|
| Sequence | ID | (Bkgd) | (Probe) | (Sample B/ |
| N→C | NO: | Signal | Signal | Sample A) |
| HDNMYLNE | 210 | 81.50 | 7,243.75 | 6.47 |
| DINFSLGI | 211 | 160.50 | 4,797.29 | 4.90 |
| NFNYLLPH | 212 | 470.56 | 13,817.90 | 4.88 |
| HRSYPRRG | 213 | 805.42 | 19,001.58 | 4.56 |
| NNNFSLHM | 214 | 199.94 | 4,374.19 | 4.45 |
| PDSYPRWG | 215 | 288.88 | 5,591.60 | 4.27 |
| EDSYPRDG | 216 | 297.83 | 5,021.94 | 4.08 |
| WANYLLPP | 217 | 182.90 | 2,860.10 | 3.97 |
| DNNYLLLL | 218 | 156.87 | 2,242.02 | 3.84 |
| WIKLKQND | 219 | 181.65 | 2,343.96 | 3.69 |
| TGKLKQDG | 220 | 209.87 | 2,464.29 | 3.55 |
| LFLLQQDD | 221 | 219.12 | 2,423.10 | 3.47 |
| NLLLKHPD | 222 | 216.15 | 2,297.98 | 3.41 |
| FFKGHFKF | 223 | 543.19 | 5,435.29 | 3.32 |
| LFKLKQDG | 224 | 277.71 | 2,776.37 | 3.32 |
| HLKLKQEN | 225 | 183.65 | 1,745.92 | 3.25 |
| LYLLKHHP | 226 | 304.92 | 2,527.00 | 3.05 |
| HYKLKQHE | 227 | 165.10 | 1,296.65 | 2.97 |
| WNSYPRND | 228 | 292.56 | 2,198.94 | 2.91 |
| LILLQQLD | 229 | 177.10 | 1,251.12 | 2.82 |
| PWKLKQDP | 230 | 197.52 | 1,339.15 | 2.76 |
| FWKGHFDF | 231 | 230.42 | 1,455.83 | 2.66 |
| FFNFSLPA | 232 | 165.50 | 1,043.90 | 2.66 |
| FYNMYLLN | 233 | 251.75 | 1,558.62 | 2.63 |
| WGKLKQGE | 234 | 209.10 | 1,231.15 | 2.56 |
| HNSYPRNE | 235 | 143.58 | 802.10 | 2.48 |
| GKSYPRHR | 236 | 553.79 | 3,049.48 | 2.46 |
| YHNYLLHP | 237 | 240.19 | 1,309.00 | 2.45 |
| AHNYLLEH | 238 | 115.46 | 618.60 | 2.42 |
| VLLHYGLY | 239 | 347.96 | 1,796.19 | 2.37 |
| LFKLKQND | 240 | 366.58 | 1,857.96 | 2.34 |
| HNNYLLNN | 241 | 190.35 | 963.60 | 2.34 |
| HRSYPRVY | 242 | 494.92 | 2,451.98 | 2.31 |
| FWNMYLNP | 243 | 158.25 | 774.75 | 2.29 |
| HFKLKQGG | 244 | 641.69 | 3,108.23 | 2.28 |
| NNSYPRHR | 245 | 731.12 | 3,422.79 | 2.23 |
| PFKLKQGN | 246 | 1,523.23 | 6,982.96 | 2.20 |
| EESYPRDK | 247 | 121.27 | 553.90 | 2.19 |
| ALNFSLNW | 248 | 152.44 | 662.15 | 2.12 |
| IFLLQQDN | 249 | 238.04 | 1,007.54 | 2.08 |
| WRSYPRRA | 250 | 619.96 | 2,611.56 | 2.07 |
| RLLHYGSY | 251 | 290.67 | 1,203.13 | 2.05 |
| HNNFSLEP | 252 | 107.88 | 441.40 | 2.03 |
| MYKLKQDN | 253 | 184.58 | 746.29 | 2.02 |
| WLLLKHGL | 254 | 222.13 | 895.21 | 2.01 |
| DRSYPRHT | 255 | 68.94 | 271.17 | 1.98 |
| YFLLQQGP | 256 | 202.29 | 793.65 | 1.97 |
| PGKGHFPI | 257 | 260.71 | 998.08 | 1.94 |
| AFNMYLDE | 258 | 115.25 | 439.62 | 1.93 |
| FVNFSLGI | 259 | 238.31 | 900.48 | 1.92 |
| RRSYPRGR | 260 | 445.58 | 1,661.87 | 1.90 |

Binding Affinity Measurement

The binding affinity (K_{D}) of more than 150 strongest-binding ligands from Iteration 1 and Iteration 2 screening were measured using a custom peptide microarray “density” chip-based experiment. Several ligands with nM range affinities were obtained. A list of 30 highest affinity ligands discovered for ESRRG is given in Table 11.

TABLE 11
A list of 30 peptide ligands displaying
highest affinity for ESRRG is shown.
Peptide sequences are N → C terminal, and
capped by an N-terminal acetyl group.

| Peptide | SEQ | |
|---|---|---|
| Sequence | ID | K_{D} |
| (N-->C) | NO: | (Molar) |
| IFSEMRVY | 261 | 7.88E−09 |
| FWKGHFDF | 262 | 9.16E−09 |
| IKQNHNNS | 263 | 1.25E−08 |
| AIRLFLMG | 264 | 1.38E−08 |
| IFSYVRVY | 265 | 1.54E−08 |
| IFSKIRVY | 266 | 1.56E−08 |
| IKQDHNNS | 267 | 1.57E−08 |
| IFSEIRVY | 268 | 1.61E−08 |
| NIKVNANN | 269 | 1.70E−08 |
| IFSYPRVY | 270 | 1.75E−08 |
| QYPKMQRL | 271 | 1.79E−08 |
| NSLKKHLL | 272 | 1.83E−08 |
| ANNIRLLH | 273 | 1.89E−08 |
| VQYNYLQR | 274 | 1.93E−08 |
| NSLLKHLL | 275 | 2.07E−08 |
| AIRLHLMG | 276 | 2.24E−08 |
| NSLRKHLL | 277 | 2.44E−08 |
| NIKLFANN | 278 | 2.47E−08 |
| SLLFHLLK | 279 | 2.67E−08 |
| VLLHYGLY | 280 | 2.74E−08 |
| AIRGALMG | 281 | 2.74E−08 |
| FYNMYLLN | 282 | 2.74E−08 |
| LLHSSKSQ | 283 | 2.83E−08 |
| LLHKSKSQ | 284 | 2.85E−08 |
| ANNLRLLH | 285 | 2.89E−08 |
| ANNSRLLH | 286 | 2.89E−08 |
| AANMFLLL | 287 | 2.90E−08 |
| LYLLKHHP | 288 | 2.91E−08 |
| NNSWRLHL | 289 | 2.94E−08 |
| ANNVRLLH | 290 | 3.05E−08 |

In Vitro Assay

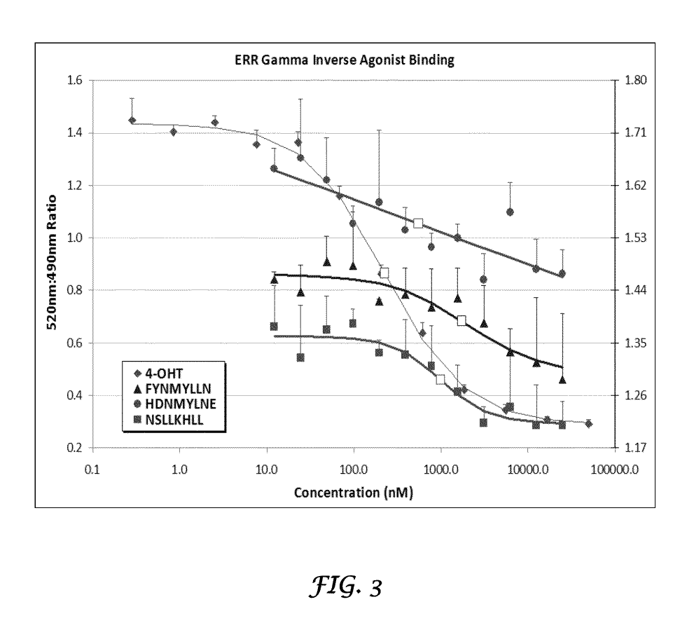

A subset (14) of highest affinity peptide ligands to ESSRG were selected to be assayed for inverse agonist activity for the interaction of ESSRG with its co-activator PGC1α using TR-FRET (time-resolved Forster Resonance Energy Transfer) (FIG. 3). Peptides were custom synthesized and their EC50 values were compared to that of 4-hydroxytamoxifen, a known inverse agonist of ESRRG with an EC50 of 220 nM. Three of the peptides tested displayed within an order of similar EC50 values to that of 4-OHT. Peptides were also analyzed for modulation of ESRRG gene transcriptional activity using a luciferase promoter-receptor assay.

### Example II

### Annexins A1 and A5

Annexins (ANX) are a family of ubiquitous, Ca²⁺ dependent, phospholipid binding cellular proteins, common to plants and animals, many of whom are involved in membrane organization, membrane traffic, and regulation of Ca²⁺ concentrations within cells. Deregulation in annexin expression and activity has been correlated with several human disease characterized as annexinopathies. Certain annexins (e.g. ANX-A1 and ANX-A5) are also proposed to be disease biomarkers, such as for lung exposure to diesel exhaust particles (Lewis, Rao et al. 2007).

All annexins share a core domain of four similar repeats, approximately 70 amino acids long, but comprise varying amino terminal regions. The vertebrate annexins share only 45-55% homology overall but display ca. 80% homology among the repeat regions, and they reveal conservation of their secondary and tertiary structures. Thus, candidate ligands (to shared regions of homology; i.e., excluding ligands to variable N-terminal regions) are expected to be shared among the annexins, using the principle applied in this approach.

Candidate ligands to Annexin A1 and A5, which share 44% overall homology, were obtained through data and textmining strategies that have been described in detail previously. The following is a brief description.

Step 1.0: Datamining

Protein Sequence

ANXA1 → 1MCX (PDB ID)
>1MCX: A|PDBID|CHAIN|SEQUENCE (320 AA)
(SEQ ID NO: 291)
MAMVSEFLKQAWFIDNEEQEYIKTVKGSKGGPGSAVSPYPTFNPSSDVEALHKAITVKGVDE
ATIIEILTKRTNAQRQQIKAAYLQEKGKPLDEALKKALTGHLEEVALALLKTPAQFDADELRA
AMKGLGTDEDTLNEILASRTNREIREINRVYKEELKRDLAKDITSDTSGDYQKALLSLAKGDR
SEDLAINDDLADTDARALYEAGERRKGTDLNVFITILTTRSYPHLRRVFQKYSKYSKHDMNK
VLDLELKGDIENCLTVVVKCATSKPMFFAEKLHQAMKGIGTRHKTLIRIMVSRSEIDMNDIKA
CYQKLYGISLCQAILDETKGDYEKILVALCGGD

Synonyms (UniProtKB)—a.k.a. ANX-1, Annexin I, ANX-V, Lipocortin I, Calpactin-2, Calpactin II, Chromobindin-9, p35, Phospholipase A2 inhibitory protein.

ANXA5 → 1AVR (PDB ID)
>1AVR: A|PDBID|CHAIN|SEQUENCE (346 AA)
(SEQ ID NO: 292)
MAQVLRGTVTDFPGFDERADAETLRKAMKGLGTDEESILTLLTSRSNAQRQEISAAFKTLFG
RDLLDDLKSELTGKFEKLIVALMKPSRLYDAYELKHALKGAGTNEKVLTEIIASRTPEELRAI
KQVYEEEYGSSLEDDVVGDTSGYYQRMLVVLLQANRDPDAGIDEAQVEQDAQALFQAGEL
KWGTDEEKFITIFGTRSVSHLRKVFDKYMTISGFQIEETIDRETSGNLEQLLLAVVKSIRSIPAY
LAETLYYAMKGAGTDDHTLIRVMVSRSEIDLFNIRKEFRKNFATSLYSMIKGDTSGDYKKAL
LLLCGEDD

Synonyms (UniProtKB)—a.k.a. ANX-5, Annexin V, ANX-V, Lipocortin V, Endonexin II, Calphobindin I, CBP-1, Placental anticoagulant protein 1 (PAP-1), Placental anticoagulant protein 4 (PP4), Thromboplastin inhibitor, Vascular anticoagulant alpha, Anchorin CII.

PSI-BLAST (ANXA5 and ANXA1)
Annexins A1-A11, A13
Annexins B9-B12
Annexins D1-D7
Annexins E1
Giardins alpha (1, 2, 4, 5, 7, 14, 19)

Step 1.1: Dataset of Homologous Proteins (DHP1)

The primary sequences of ANXA1 and ANXA5 were ‘BLAST’ed iteratively using PSI-BLAST with the following parameters: E (expectation threshold)—0.001 (0.005 for PSI BLAST); Max. target sequences—500; with all other parameters (word size, matrix, etc.) at ‘default’, against the NR (non-redundant protein) database.

Identical set of sequence homologous proteins were found through iterative PSI-BLAST (total of 3 iterations). The proteins with sequence homologies to ANXA1 and ANXA5 are given in the above table. These homologous sequence proteins are used in subsequent text mining searches (Step 2 below). The text mining searches identify words, phrases, and text containing the names of the homologous sequence proteins co-occurring with words, phrases, text, abbreviations, or annotation indicative of peptide sequences and peptide ligands.

Step 1.2: InterPro (DHP2)

An InterPro query for ANXA1 and ANXA5 was carried out starting from its FASTA sequence (i.e., using InterProScan). Both proteins comprise a common ‘annexin repeat’ region, as inferred earlier. Various members of the annexin family were found as children of the parent Annexin—ANXA 1, 3, 5, 13, etc.

| InterPro ID | Protein Name | |
|---|---|---|
| IPR001464 | Annexin | Parent |
| IPR018502 | Annexin repeat | Contains |

Step 1.3: IntAct/DIP (Database of Complementary ‘Interacting’ Proteins)

IntAct is an open source database system and analysis tools for protein interaction data. An IntAct database search was performed. Proteins known to interact with ANXA1 and ANXA5 are listed below.

ANXA5—DNA methyltransferase 1, DNMT1; basophilic leukemia-expressed protein; ATP-dependent helicase ATRX; translation initiation factor, IF-2; farnesyl diphosphate farnesyltransferase, FPP.

ANXA1—ubiquitin thioesterase 25; protein phosphatase 2C; beta-actin; glucose transporter type 5, Glut4; IKBA, I-kappa-B-alpha, major histocompatibility complex enhancer-binding protein, MAD3.

Other annexin family proteins interact with F-actin, profiling, non-erythroid spectrin (fodrin) calspectin, synapsin, GFAP, etc.

This information on interacting proteins is used in subsequent text mining (Step 2 below). Text mining selectively identifies words, phrases, or text containing the names of the interaction proteins, co-occurring in text containing words, phrases, abbreviation, or annotation associated with peptide amino acid sequences or peptide ligands. By this process information is obtained that indicates possible amino acid sequences involved in interactions with ANXA1 and ANXA5. From this sequence, information about candidate peptide ligands able to interact with ANXA1 and ANXA5 will be sourced.

Step 2: Textmining (PubMed Central)

Text mining is performed where the search terms include the proteins and homologs identified in Step 1.0 along with co-occurring words, text, phrases, or abbreviations that designate amino acid sequences and peptide ligands; e.g., peptide, ligand, motif, sequence, fragment, region, epitope, paratope, amino acid, etc.

For example, a PubMed query using the terms ‘Annexin and Peptide and Ligand’ was carried out, which leads to several references including a review on ‘Annexins’ (Genome Biology, 2004). (Moss and Morgan 2004) Perusal of the journal article further provided proteins that interact with Annexins (a subset of those also obtained through a query of the IntAct database). A subsequent search of PubMed using combination keywords was conducted. For example,

1. (Annexin A5) and Collagenase Type II) and binding (or peptide/ligand/motif/epitope/region/sequence, etc.);

2. (Annexin A7) and Sorcin and binding (or peptide/ligand/motif/epitope/region/sequence, etc.); and so on.

Ensuing literature was curated manually for references to peptide sequences, motifs, etc.; or referrals to other literature containing the same. A non-exhaustive list of the candidate ligand table for Annexin A1 and Annexin A5 is shown (Table 12).

The same candidate ligand set was used for ANXA1 and ANXA5 screening, due to significant shared homology and homologous proteins between Annexin family proteins.

TABLE 12
A list of candidate ligands circumscribing the ligand space for annexins
ANXA1 and ANXA5 is shown. The source of the ligand sequence along with
its literature reference, obtained through combination keyword queries
specific for the protein, is indicated. Peptides were obtained through
careful manual curation of this literature. Redundant (highly similar
or contained/entrained) sequences were culled using a multiple alignment
program; the larger sequence was invariably retained. The SOQ ID NOS of
the candidate ligands are indicated in parentheses.

| Candidate Ligand (SEQ ID NO:) | Source | Literature Reference |
|---|---|---|
| MEKEFPGFLENQKDPL (293) | S100A10 (similar seq. in | FEBS J. 2007, PMID: |
| | S100A11 and Dicalcin) | 17714509 |
| PPAETELQAM (294), | IgG binding domains of | JBC 2001, PMID: |
| LPSAEVELQAK (295) | FPRL1, FPRL2, and FPR | 15187149 |
| PGAYPGXXX (296) | Galectin 3 | BBA 2004, PMID: |
| | | 15238251 |
| LAIACHDSFIKSTQK (297) | S100C-ANXA1 | Biochem, J. 1996, |
| | | PMID: 8870658 |
| LTIACNDYFVVHMKQK-GKK (298) | ANXA2-p11 protein | Gene 1992; PMID: |
| | | 1533380 |
| MASPLDQ (299), | ANXA11-calcyclin | BBRC 1993; PMD: |
| MASPLDQAIG (300) | | 8250893 |
| WQQYEPNGKGT (301), | ANXA6-SPA (surfactant | Biochem. J. 1999; |
| WKKDEPNDHGS (302) | protein-A) | PMID 10377263 |
| AEVKKPVEAAAPG (303), | Rat HSP47-ANXA5 | Glycobiol, 2005, |
| VRSLSNTANVTWKL (304), | | PMID: 16000695, |
| FLVRDNQSGSL (305) | | also see PMID |
| | | 8667030 |
| QMAGGFDEKAGGAQLGVMQ (306) | N-Telopeptide of | Conn. Tiss. Res. 2003, |
| | Collagen-ANXA5 | PMID: 14660093 |
| ALADEREAVQKKTFTKWVN (307) | Calspectin (brain spectrin | JBC 1994, 269(26), |
| | aka fodrin)-ANXA6 | 17656-62. PMID: 8021276 |
| YLSEKKGGNGKN (308) | Angiostatin sequence | Microvasc. Res. 2002, |
| | (binds ANXA2) | PMID: 12453439 |
| DEAAGNLRQHDAEVDATLKSLNNQ | Chondrocalcin-ANXA5 | Biochem. J. 1986, 237, |
| (309) | | 923-25. PMID: 3800925; |
| | | PMCID: 1147077 |
| FQSERSRARYEMAS (310) | αᵥβ₅-integrin-ANXA5 | Mol. Cell, 2003, |
| | | PMID: 12769841 |
| YFVVHMKQKGKK (311) | ANXA2-binding site | JBC 1992, |
| | on p11 protein | PMID 1385811 |
| YLSEKKGGNGKN (312) | Angiostatin sequence | Microvasc. Res. 2002, |
| | (binds ANXA2) | PMID: 12453439 |
| NSIAYLDEAAGNLK (313), | Chondrocalcin-ANXA5 | Biochem. J. 1986, 237, |
| SGDYWIDPNQG (314) | | 923-25. PMID: 3800925; |
| | | PMCID: 1147077 |
| KEATSTFTNITYRGT (315) | Integrin beta-3 | PMID 9065427; |
| | cytoplasmic | PMCID 78593, PepLib |
| | domain/ANXA5 | 124823 |

Step 3: Candidate Ligand Selection for Array

A subset of ligands (Table 13) was selected for automated sequence expansion and peptide microarray production, as previously described. Only the core 2 amino acids were permuted to create an 8-mer superlibrary.

TABLE 13
A subset of candidate ligands from Table 12
was selected for ligand walking, sequence
expansion, and array based affinity matura-
tion for annexins A5 and A1. Underlined
sequences in bold correspond to the sources
from which highest affinity binders were
obtained through chip-based maturation.
List of ANXA1 and ANXA5 candidate ligands
(SEQ ID NOS.)

| | |
|---|---|
| QMAGGFDEKAGGAQLGVMQ | MEKEFPGFLENQKDPL |
| (306) | (293) |
| DEAAGNLRQHDAEVDATLKSLNNQ | LAIACHDSFIKSTQK |
| (309) | (297) |
| SGDYWIDPNQG | YFVVHMKQKGKK |
| (314) | (311) |
| NSIAYLDEAAGNLK | MASPLDQAIG |
| (313) | (300) |
| FQSERSRARYEMAS | WQQYEPNGKGT |
| (310) | (301) |
| VRSLSNSTARNVTWKL | ALADEREAVQKKTFTKWVN |
| (342) | (307) |
| FLVRDNQSGSL (305) | |

Step 4: Screening and Data Analysis

Study 1: The intent of this study was to demonstrate that a shared, customized ligand space shared by closely similar proteins may be used to obtain distinct ligands for those proteins. The example provided examines the case of ANXA1 and ANXA5.

Both ANXA1 and ANXA5 were directly labeled with a fluorescent tag (Cy 3 and Cy 5, respectively) and independently screened against a custom microarray (ca. 3900) of 8-mer peptides (from ligand walking of ANXA5 candidate ligands) and their permutations generated from the substitution matrix (Table 14). Due to the expected presence of these proteins in serum, each protein screening was carried out in the presence of a 100-fold excess of BSA that simulated a complex background of proteins.

TABLE 14
Matrix of permutations used for the initial array-based screening
experiments of candidate peptide ligand permutations for ANXA5.

| Code | Definition (Grouped AAs) |
|---|---|
| A | lipo-bulky-V I L M |
| B | lipo-small-G A V F P |
| C | +charged-R K H G |
| D | −charged-D E H A |
| E | Polar-N Q M C |
| F | Aromatic-Y F H W |
| G | H donor-Y S T C |
| H | amide, carboxyl-N Q D E |

The results of hybridization of fluorescently labeled ANXA1 and ANXA5 against an identical peptide microarray are shown below. Input sequences clearly did not favor any single amino acid (FIG. 4A). Several (ca. 400-600) high-affinity ligands were obtained for ANXA1 and ANXA5. The results indicate clearly that divergent sequence space of high-affinity ligands to ANXA1 (FIG. 4B), different from that for ANXA5 (FIG. 4C), could be identified from primary screening.

Iterative screening for affinity maturation is underway. It is anticipated that affinity maturation will provide high-affinity peptide ligands that can selectively and specifically bind/detect/modulate only ANXA1 or ANXA5.

Study 2: On-chip binding affinity measurements were carried out for a set of highest-binding Iteration 1 (Iter 1) ligands to ANXA5 from the primary screening experiment. A range of affinities, from nM to low mM, were obtained. The sequences showing the highest and their affinities are displayed in Table 15. The lineage of all Iter 1 ligands could be traced easily to their respective parents. The two families of highest Iter 1 binders came from two interacting proteins of ANXA5; chondrocalin and _{v 5}-integrin (see Table 15). Iterative array generation and screening is anticipated to lead to further affinity maturation to provide Iter 2 (and higher generation) ligands with nM to sub-nM affinities.

TABLE 15
Binding affinity (K_{D}) of Iteration 1
ligands to ANXA5 from primary screening
of a custom designed peptide microarray,
determined on a peptide density-array
chip. High-affinity peptide ligands from
two families are illustrated below.
SEQ ID NOS: are indicated in parenthesis.

| NSIAYLDEAAGNLK (313) | | FQSERSRARYEMAS (310) | |
|---|---|---|---|
| (Chondrocalcin/ANXA5) | | αvβ₅-integrin/ANXA5) | |
| Ligand | K_{D} | Ligand | K_{D} |
| AYLHYAAG (316) | 21.9 nM | SERYTARY (323) | 86.9 nM |
| AYLGYAAG (317) | 24.5 nM | SERYSARY (324) | 82.5 nM |
| AYLGHAAG (318) | 57.5 nM | SERYWARY (325) | 91.7 nM |
| AYLMHAAG (319) | 63.0 nM | SERCWARY (326) | 218 nM |
| AYLHHAAG (320) | 87.6 nM | SERCYARY (327) | 301 nM |
| AYLVHAAG (321) | 92.2 nM | SERYYARY (328) | 361 nM |
| AYLHGAAG (322) | 93.1 nM | SERFFARY (329) | 520 nM |

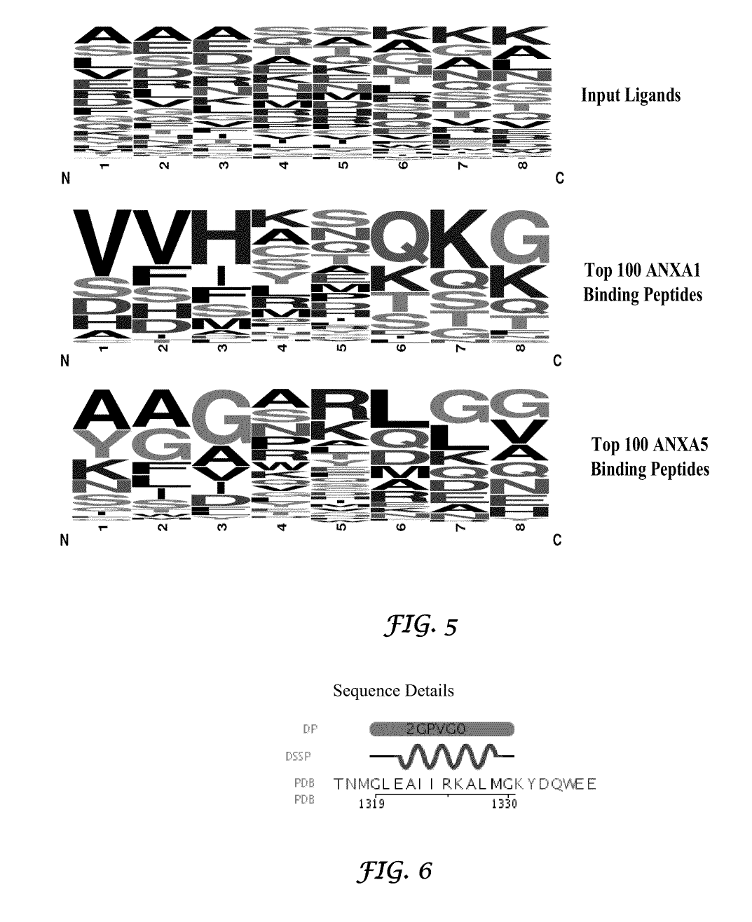

Study 3: The methodology could be used for candidate ligand generation using HALO/Gen and direct competitive screening of a customized peptide ligand array comprising shared candidate ligands to highly sequence similar proteins within a family (e.g., annexins, estrogen receptors (ERs), ERRs, interleukins) in order to evolve selective or highly-specific high-affinity ligands to such proteins. In this example, ANXA1 and ANXA5, orthogonally labeled with Cy-3 and Cy-5 fluorescent tags, were screened simultaneously against a custom ligand microarray of ca. 13,000 ligands generated from Tier-1 permuted sequence combinations of the entire candidate ligand library for annexins (Table 13). The results (consensus chart of high affinity ligand motifs) of the competitive screening study are demonstrated in FIG. 5. Sets of high-affinity peptide sequences (and motifs) specific for either protein were obtained, from only a single microarray, along with certain shared motifs that were selective for one or the other. Further evolution of specificity and selectivity is expected from iterative screening (either singly or competitively) of Gen 1 high-affinity ligands and their permutations.

The studies described hereinabove validate the methodology of the present invention. Proteins could be datamined for homology and textmined for candidate ligands. Array screening allowed parallel interrogation of several thousand rationally designed peptide ligands leading to the discovery of several high-affinity, high value candidates, which are expected to have multiple applications including in drug discovery and as antibody replacements. Since the design of the customized ligand space sampled through screening stems only from knowledge of protein sequence, it will be possible by this approach to derive high affinity peptide ligands to even proteins whose structures are not available (e.g., G-Protein Coupled Receptors, GPCRs). This technology offers the potential to very quickly generate lead candidates for major disease protein targets.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

For the purposes of this invention, proteins refer to molecules that possess an amino acid sequence that matches or partially matches the amino acid sequence found in the target or query protein an “homologous protein.”

Peptide is used to denote an oligomer in which the monomers are alpha amino acids joined together through amide bonds. A “peptide” can also be referred to as a “polypeptide”. In the context of this invention, one should appreciate that the amino acids may be the L-optical isomer or the D-optical isomer.

Standard single letter abbreviations for amino acids are used (e.g., P for proline). When incorporated into a protein, an amino acid is referred to as an “amino acid residue”. The term “protein” is understood to include the terms “polypeptide” and “peptide” (which, at times, may be used interchangeably herein) within its meaning. The polypeptide's “primary structure” is the particular amino acid sequence of a given protein written from the amino-terminus to carboxy-terminus.

As is the case for all proteins, the precise chemical structure depends on a number of factors. As ionizable amino and carboxyl groups are present in the molecule, a particular protein may be obtained in an acidic or basic salt, or in a neutral form. All such forms are included in the definition.

Furthermore, the primary amino acid sequence may be augmented by derivatization using sugar moieties (glycosylation) or by other supplementary molecules such as lipids, phosphate, acetyl groups, and the like. The primary amino acid structure may also aggregate to form complexes. In any event, such modifications are included in the definition.

Also, individual amino acid residues in the chain may be modified by oxidation, reduction, or other derivatization, and the protein may be cleaved to obtain fragments which retain binding activity. Such alterations which do not destroy binding activity do not remove the protein sequence from the definition,

In the case of naturally occurring proteins, an amino acid residue's R group differentiates the 20 amino acids from which proteins are synthesized, although one or more amino acid residues in a protein may be derivatized or modified following incorporation into protein in biological systems (e.g., by glycosylation and/or by the formation of cystine through the oxidation of the thiol side chains of two non-adjacent cysteine amino acid residues, resulting in a disulfide covalent bond that frequently plays an important role in stabilizing the folded conformation of a protein, etc.).

As those in the art will appreciate, non-naturally occurring amino acids can also be incorporated into proteins, particularly those produced by synthetic methods, including solid state and other automated synthesis methods. Examples of such amino acids include, without limitation, a-amino isobutyric acid, 4-amino butyric acid, L-amino butyric acid, 6-amino hexanoic acid, 2-amino isobutyric acid, 3-amino propionic acid, ornithine, norlensine, norvaline, hydroxyproline, sarcosine, citralline, cysteic acid, t-butylglyine, t-butylalanine, phenylylycine, cyclohexylalanine, β-alanine, fluoro-amino acids, designer amino acids (e.g., β-methyl amino acids, α-methyl amino acids, N-α-methyl amino acids) and amino acid analogs in general.

In addition, when an α-carbon atom has four different groups (as is the case with the 20 amino acids used by biological systems to synthesize proteins, except for glycine, which has two hydrogen atoms bonded to the a carbon atom), two different enantiomeric forms of each amino acid exist, designated D and L. In mammals, only L-amino acids are incorporated into naturally occurring polypeptides. Of course, the instant invention envisions proteins incorporating one or more D and L-amino acids, as well as proteins comprised of just D or L-amino acid residues.

To confer resistance to proteolysis, peptide bonds may also be substituted by retro-inverso pseudopeptide bonds. According to this modification, the amino acid sequence of the modified peptide may be identical to the sequence of the native L-amino acid sequence, except that one or more of the peptide bonds are replaced by a retro-inverso pseudopeptide bond. The synthesis of peptides with one or more reduced retro-inverso pseudopeptide bonds is known in the art.

A protein is “synthetic” when produced by in vitro chemical (e.g., solid phase peptide synthesis) or enzymatic synthesis. The peptides of this invention, including analogs and other modified variants may generally be prepared following known techniques. Preferably, synthetic production of the peptides of the invention may be according to the solid phase synthesis method. Alternatively, peptides of this invention may be prepared in recombinant systems using polynucleotide sequences encoding the peptides. It is understood that a peptide of this invention may contain more than one of the above described modifications within the same peptide.

Herein, the following abbreviations may be used for the following amino acids (and residues thereof): alanine (Ala, A); arginine (Arg, R); asparagine (Asn, N); aspartic acid (Asp, D); cyteine (Cys, C); glycine (GIy, G); glutamic acid (GIu, E); glutamine (GIn, Q); histidine (His, H); isoleucine (He, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V). Non-polar (hydrophobic) amino acids include alanine, leucine, isoleucine, valine, proline, phenylalanine, tryptophan, and methionines. Neutral amino acids include glycine, serine, threonine, cysteine, tyrosine, esparagine, and glutamine. Positively charged (basic amino acids include arginine, lysine and histidine. Negatively charged (acidic) amino acids include aspartic acid and glutamic acid.

In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins”.

A peptidomimetic is a molecule that mimics the biological activity of a peptide but is no longer peptidic in chemical nature. By strict definition, a peptidomimetic is a molecule that no longer contains any peptidic bonds. However, the term peptidomimetic is sometimes used to describe molecules that are no longer completely peptidic in nature, such as pseudo-peptides, semi-peptides, and peptoids. Whether completely or partially non-peptide, peptidomimetics according to this invention provide a spatial arrangement of reactive chemical moieties that closely resembles the three-dimensional arrangement of active groups in the peptide on which the peptidomimetic is based. As a result of the similar active-site geometry, the peptidomimetic has effects on biological systems which are similar to the biological activity of the peptide.

The present invention encompasses peptidomimetic compositions which are analogs that mimic the activity of biologically active peptides according to the invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Adessi, C. and C. Soto (2002). “Converting a peptide into a drug: strategies to improve stability and bioavailability.” Current Medicinal Chemistry 9(9): 963-978.
Allegretti, M., R. Bertini, et al. (2008). “Allosteric inhibitors of chemoattractant receptors: opportunities and pitfalls.” Trends Pharmacol. Sci. 29(6): 280-286.
Anderson, E. and R. B. Clarke (2004). “Steroid receptors and cell cycle in normal mammary epithelium.” J Mammary Gland Biol Neoplasia 9(1): 3-13.
Ariazi, E. A. and V. C. Jordan (2006). “Estrogen-related receptors as emerging targets in cancer and metabolic disorders.” Current Topics in Medicinal Chemistry 6(3): 203-215.
Arkin, M. R. and J. A. Wells (2004). “Small-molecule inhibitors of protein-protein interactions: Progressing towards the dream.” Nature Rev. Drug Disc. 3(4): 301-317.
Christensen, D. J., E. B. Gottlin, et al. (2001). “Phage display for target-based antibacterial drug discovery.” Drug Disc. Today 6(14): 721-727.
Clarke, R. B., E. Anderson, et al. (2004). “Steroid receptors in human breast cancer.” Trends Endocrinol Metab 15(7): 316-23.
Dias, R. L. A., R. Fasan, et al. (2006). “Protein Ligand Design; From Phage Display to Synthetic Protein Epitope Mimetics in Human Antibody Fc-Binding Peptidomimetics.” J. Am. Chem. Soc. 128(8): 2726-2732.
Dumont, J. A., A. J. Bitonti, et al. (1996). “Progression of MCF-7 breast cancer cells to antiestrogen-resistant phenotype is accompanied by elevated levels of AP-1 DNA-binding activity.” Cell Growth Differ 7(3): 351-9.
Dutton, G. (2007). “Development of peptide drugs advances briskly.” Gen. Engg. Biotech. News 27(10).
Galindo, C. L., J. F. McCormick, et al. (2010). “A long AAAG repeat allele in the 5′ UTR of the ERR-gamma gene is correlated with breast cancer predisposition and drives promoter activity in MCF-7 breast cancer cells.” Breast Cancer Res Treat.
Galindo, C. L., J. F. McCormick, et al. (2010). “Global germline and tumor microsatellite patterns are cancer biomarkers.” Manuscript in preparation.
Henikoff, S. and J. G. Henikoff (1992). “Amino acid substitution matrices from protein blocks.” Proc. Natl. Acad. Sci. U.S.A. 89(22): 10915-9.
Hurtado, A., K. A. Holmes, et al. (2008). “Regulation of ERBB2 by oestrogen receptor-PAX2 determines response to tamoxifen.” Nature 456(7222): 663-666.
Johnston, S. R., B. Lu, et al. (1999). “Increased activator protein-1 DNA binding and c-Jun NH2-terminal kinase activity in human breast tumors with acquired tamoxifen resistance.” Clin Cancer Res 5(2): 251-6.
Lewis, J. A., K. M. Rao, et al. (2007). “Proteomic analysis of bronchoalveolar lavage fluid: effect of acute exposure to diesel exhaust particles in rats.” Environ Health Perspect 115(5): 756-63.
Lewis, J., S. Ossowski, et al. (2006). “Text similarity: an alternative way to search MEDLINE.” Bioinformatics 22(18): 2298-2304.
McEntyre, J. R. and T. J. Gibson (2004). “Patterns and clusters within the PSM column in TiBS, 1992-2004.” Trends Biochem. Sci. 29(12): 627-633.
Moss, S. E. and R. O. Morgan (2004). “The annexins.” Genome Biol 5(4): 219.
Norman, T. C., D. L. Smith, et al. (1999). “Genetic Selection of Peptide Inhibitors of Biological Pathways.” Science 285(5427): 591-595.
Pawson, T. and J. D. Scott (1997). “Signaling Through Scaffold, Anchoring, and Adaptor Proteins.” Science 278(5346): 2075-2080.
Pini, A., A. Giuliani, et al. (2005). “Antimicrobial Activity of Novel Dendrimeric Peptides Obtained by Phage Display Selection and Rational Modification.” Antimicrob. Agents Chemother. 49(7): 2665-2672.
Riggins, R. B., J. P.-J. Lan, et al. (2008). “ERR{gamma} Mediates Tamoxifen Resistance in Novel Models of Invasive Lobular Breast Cancer.” Cancer Res 68(21): 8908-8917.
Schiff, R., P. Reddy, et al. (2000). “Oxidative stress and AP-1 activity in tamoxifen-resistant breast tumors in vivo.” J Natl Cancer Inst 92(23): 1926-34.
Sillerud, L. O. and R. S. Larson (2005). “Design and structure of peptide and peptidomimetic antagonists of protein-protein interaction.” Current Protein & Peptide Science 6(2): 151-169.
Tao, J., P. Wendler, et al. (2000). “Drug target validation: Lethal infection blocked by inducible peptide.” Proc. Natl. Acad. Sci. U.S.A 97(2): 783-786.
Vagner, J., H. Qu, et al. (2008). “Peptidomimetics, a synthetic tool of drug discovery.” Current Opinion in Chemical Biology 12(3): 292-296.
Witt, K. A., T. J. Gillespie, et al. (2001). “Peptide drug modifications to enhance bioavailability and blood-brain barrier permeability.” Peptides 22(12): 2329-2343.
Zhou, Y., C. Yau, et al. (2007) “Enhanced NF kappa B and AP-1 transcriptional activity associated with antiestrogen resistant breast cancer.” BMC Cancer 7: 59.

What is claimed is:

1. A method for discovery of one or more high-affinity peptide ligands comprising:
obtaining an amino acid sequence of at least a portion of a target protein;
identifying one or more homologous proteins of the target protein;
identifying complementary proteins, wherein the complementary proteins are proteins that bind to the target or homologous proteins;
extracting from literature or from one or more databases, peptides that have a ligand binding probability for the target protein or homologous proteins;
generating a library of one or more candidate peptide ligands from the extracted literature; and
determining if any of the one or more candidate peptide ligands binds to the target protein.

2. The method of claim 1, further comprising the steps of:
performing a “sequence modulation,” wherein subsequences of the one or more candidate peptide ligand sequences each one corresponding to an n-residue long contiguous sequence are selected to yield a super-library of n-mer parent peptides (the “n-mer superlibrary”); and
substituting one or more residues in each peptide in the n-mer superlibrary with other amino acids, to yield a “substituted n-mer superlibrary.”

3. The method of claim 2, wherein the one residue in each peptide in the n-mer superlibrary is sequentially substituted by a scanning amino acid.

4. The method of claim 3, wherein the scanning amino acid is alanine.

5. The method of claim 2, wherein the amino acid substitutions are knowledge-based.

6. The method of claim 5, wherein the knowledge-based amino acid substitutions are selected using a substitution matrix.

7. The method of claim 6, wherein the substitution matrix is a member of the BLOSUM (BLOcks of Amino Acid SUbstitution Matrix) family of substitution matrices, PAM or any custom generated matrix from data analysis.

8. The method of claim 2, wherein n is 8.

9. The method of claim 2, wherein two, four, six, or all n residues are substituted.

10. The method of claim 2, wherein the two substituted residues occupy central positions in the peptide.

11. The method of claim 2, wherein one or more central amino acids of the peptide are kept unchanged while pairs of outlying amino acids in the sequence are permuted.

12. The method of claim 2, wherein the amino acid residues in positions 1 and 8 are substituted.

13. The method of claim 2, wherein the amino acid residues in positions 2 and 7 are substituted.

14. The method of claim 2, wherein the substitutions are random variations of the peptide chemistry.

15. The method of claim 2, further comprising the step of “screening” peptides selected from the library of candidate peptide ligands, the n-mer superlibrary, and the substituted n-mer superlibrary for binding ability to the target protein using one or more experimental methods.

16. The method of claim 15, further repeating the steps of “sequence modulation” and “screening” iteratively to identify the amino acids which contribute to optimal binding of peptides selected from the library of candidate peptide ligands, the n-mer superlibrary, and the substituted n-mer superlibrary to the target protein, and thereby selecting candidate peptide ligand sequences having optimized binding activity.

17. The method of claim 15, wherein the optimized binding activity comprises affinity (Kd), specificity, selectivity, in vitro and in vivo availability, viability, and combinations and modifications thereof.

18. The method of claim 17, wherein the affinity may be modulated by dimerization, oligomerization, multivalent display, or any combinations thereof to facilitate multiplex binding.

19. The method of claim 15, wherein the binding ability is evaluated using an established assay method selected from the group consisting of FRET (Fluorescence Resonance Energy Transfer), spectrophotometric assay, fluorescence assay, NMR, MS, electrical/electronic (e.g., FET, SPR) assays, in silico docking, microarray-based analysis, pep-spot analysis, combinatorial libraries, phage display, cell based assays, and combinations or modifications thereof.

20. The method of claim 15, wherein a collection of peptides selected from the library of candidate peptide ligands, the n-mer superlibrary, and the substituted n-mer superlibrary is screened using high-throughput peptide microarrays selected from the group consisting of chip based assays, liquid arrays comprising flow cytometry bead assays, multiplex labeled beads on a substrate, or any combinations thereof.

21. The method of claim 20, wherein the collection of peptides is synthesized on the microarray chip substrate.

22. The method of claim 21, wherein the peptides are synthesized on a microarray chip substrate using a digitally controlled light source, in situ laser printing, PEPspot and other pre-synthesized peptide spotting technologies or any combinations thereof.

23. The method of claim 20, wherein about 4,000 to 2,000,000 peptides are arrayed on the chip.

24. The method of claim 15, wherein the screening of peptides selected from an initial iteration or a subsequent iteration of a library of candidate peptide ligands, its respective n-mer superlibrary, and its substituted n-mer superlibrary includes:
determining the binding affinity of ligands in vivo or in vitro by linking to a contrast agent or a detection agent, wherein the contrast agent or the detection agent is selected from the group consisting of a radioactive tracer, a GFP, a fluorophore, a quantum dot, a nanoscale structure or a nanowire;
determining in vitro modulatory (e.g., agonist, inverse agonist, antagonist) ability of ligands towards one or more agonists, inverse agonists, or antagonists; and,
performing cell-based activity assays, in vivo pharmacological assays, drug sensitivity assays, clinical/preclinical testing, or any combinations thereof.

25. The method of claim 1, wherein the target protein or fragment thereof is involved in, but not limited to, peptide-protein or protein-protein or a protein-small molecule interactions.

26. The method of claim 1, wherein the peptide ligand sequences bind to and otherwise modulate, activate, or inhibit the function of the target protein, homologous proteins, or DNA.

27. The method of claim 1, where peptide ligands sequences can be polyvalently linked or displayed for protein, DNA, and cell capture.

28. The method of claim 1, where peptide ligands can be conjugated to a payload such as a drug, protein or other molecules such as, but not limited to, liposomes, dendrimers, or combinations thereof for drug delivery.

29. The method of claim 1, wherein the target protein is selected from the group consisting of cell membrane receptors, nuclear membrane receptors, cytoplasmic, nuclear, and mitochondrial proteins, secreted proteins, circulating peptide and non-peptide receptors, membrane and circulating transporters, enzymes, chaperonins and chaperonin-like proteins, antibodies, and surface and intracellular proteins of infectious agents.

30. The method of claim 1, wherein identifying one or more homologous protein is performed using the target protein primary sequence or a fragment thereof to query one or more suitable databases with one or more suitable homology search tools, such that proteins homologous to the target protein are identified.

31. The method of claim 30, wherein the query protein amino acid sequences are obtained by translating nucleic acid sequences.

32. The method of claim 30, wherein the homologous sequences have significant sequence similarity to the target sequence as judged by an alignment score below a threshold of statistical significance.

33. The method of claim 30, wherein one or more of the homology search tools include sequence to sequence comparison (pairwise sequence alignment) done by an algorithm.

34. The method of claim 33, wherein the algorithm is selected from the group consisting of Smith-Waterman, Needleman-Wunsch, BLAST, PSI-BLAST, PHI-BLAST, WU-BLAST2, BLAT, and FASTA.

35. The method of claim 30, wherein one or more of the homology search tools is based on sequence to profile comparison (sequence-profile method) method.

36. The method of claim 35, wherein the profile method is Position Specific Scoring Matrix (PSSM)-based.

37. The method of claim 35, wherein the profile method is PSI-BLAST.

38. The method of claim 35, wherein the profile method is a Hidden Markov Model (HMM) based method.

39. The method of claim 35, wherein the profile method is HHMER or SAM.

40. The method of claim 30, wherein one or more of the homology search tools is based on profile to profile comparison (profile-profile) method.

41. The method of claim 40, wherein the profile-profile method is selected from the group consisting of FFAS, ORFeus, COMPASS, COACH, and HHpred

42. The method of claim 30, wherein one or more of the homology search tools includes protein domain comparison tools.

43. The method of claim 42, wherein the domain comparison tools include CDD, CDART, RPS-BLAST, HMMER, or IprScan.

44. The method of claim 42, wherein the domain comparison tools are applied to search for homology between the target protein primary sequence or a fragment thereof and protein domain stored in an individual protein domain databases.

45. The method of claim 44, wherein the individual domain database is selected from the group consisting of Pfam, SMART, PROSITE, Propom, PRINTS, UniProt, TIGRFAMs, PIR-SuperFamily, and SUPERFAMILY.

46. The method of claim 42, wherein the domain comparison tools are applied to search for homology between the target protein primary sequence or a fragment thereof and protein domain stored in a protein domain meta-database.

47. The method of claim 46, wherein the meta-database is InterPro, CDD or CDART.

48. The method of claim 30, wherein one or more of the homology search tools includes protein domain architecture comparison tools.

49. The method of claim 48, wherein the domain architecture comparison tool is CDART.

50. The method of claim 1, wherein the identification of peptides that have a protein or DNA binding probability includes functional domain prediction bioinformatic methods from the group consisting of ab-initio domain predictors, secondary structure predictors, disorder predictors, linker predictors, gene fusion methods, domain co-occurrence methods, genetic context methods (gene neighborhoods, gene clusters and operons), phylogenomic profiles, and metabolic reconstruction.

51. The method of claim 1, comprising the further step of identifying “complement proteins” wherein sequences selected from group consisting of the target protein, the DNA, the homologous proteins, and fragments thereof are queried against one or more protein-protein interaction databases, such that proteins known to interact with the query sequences are identified.

52. The method of claim 51, wherein the protein-protein interaction databases queried include DIP IntAct and DOMINO, and all other open-source computational or manually curated databases.

53. The method of claim 1, wherein the identification of peptides with a ligand binding probability is performed using textmining, such that candidate peptide ligands likely to bind to the target protein, candidate peptide ligands likely to bind to the DNA, candidate peptide ligands likely to bind to homologous proteins, and candidate peptide ligands present in the sequence of complement proteins that are likely to bind to the target protein or to homologous proteins are identified.

54. The method of claim 53, wherein the textmining includes mining and curating ligand-binding data collected from scientific literature and protein-ligand databases, publications, literature reports, documents, computerized records, abstracts, scientific journals, and other public and non-public sources (“data sources”).

55. The method of claim 53, wherein textmining is performed manually.

56. The method of claim 53, wherein textmining is computer-assisted.

57. The method of claim 56, wherein the computer-assisted textmining is performed using one or more text similarity search engines, wherein the search engine comprises eTBLAST, Contaro, the search engine and database HALO, or any combinations or modifications thereof.

58. The method of claim 53, wherein textmining is both manual and a computer-assisted.

59. The method of claim 53, wherein candidate peptide ligands are identified by text searching publicly accessible databases using customized search terms, for references including peptide ligands and interacting sequences, epitopes/paratopes, motifs, regions, and/or domains to the target protein and/or to homologous proteins.

60. The method of claim 53, wherein input text used to search a database for relevant protein-ligand interactions in the protein synopsis is selected from the group consisting of, but not limited to, PubMed, UniProt, ChemAbstracts, PDB, or InterPro.

61. The method of claim 53, wherein the textmining is performed by querying “data sources” with one or more protein names, synonyms, commonly accepted acronyms, database accession numbers, domain names, E.C. numbers, or another non-sequence suitable identifiers of the target protein, homologs, or complement proteins.

62. The method of claim 61, wherein the BioMint, UniProt, PDB databases or combinations thereof are employed to obtain protein synonyms.

63. The method of claim 53, wherein textmining is performed by querying “data sources” with permutations and combinations of search terms including sequences or fragments of the target protein, homologs, or complement proteins; protein names; synonyms; commonly accepted acronyms; database accession numbers; domain names; E.C. numbers; or other suitable identifiers.

64. The method of claim 63, wherein the search terms include non-identifier keywords.

65. The method of claim 53, wherein the candidate peptide ligands are ranked according to the degree of sequence homology between the primary sequence of the homologous protein used for the textmining query and the primary sequence of the target protein, wherein ligands corresponding to proteins with higher homology scores are ranked highest.

66. The method of claim 53, wherein the evaluation of ligand binding probabilities is performed by querying a peptide database.

67. The method of claim 66, wherein the peptide database is PepLib, LiBase or other databases.

68. The method of claim 1, wherein the library of candidate peptide ligand sequences is culled of redundant or “entrained” sequence using a Multiple Sequence Alignment (MSA) algorithm, such that when two sequences are identical or almost identical, only the longest of the two sequences is selected.

69. The method of claim 68, wherein the MSA algorithm is ClustalW2.

70. The method of claim 68, wherein the MSA algorithm is selected from the group consisting of ClustalW, DCA, Dialign, POA, T-Coffee, MAFFT, and MUSCLE.

71. The method of claim 1, wherein the target protein, homologous protein, complementary protein or peptide ligand binds a nucleic acid.

72. A computer program embodied on a computer readable medium to perform the method of claim 1.

73. A computer system, comprising programming to perform the method of claim 1.

74. A computer storage media, comprising programming to perform the method of claim 1.

75. A composition comprising one or more novel peptides capable of binding to the target protein, DNA, or both identified by the method of claim 1.

76. An optimized binding agent or binding agent composition from multiple peptides discovered by the method of claim 1 or are otherwise identified, wherein the agent targets several different protein domains and comprises several short peptides that are linked.
